(19) United States
(12) Patent Application Publication
(10) Pub. No.: US 2003/0105430 A1
(43) Pub. Date: Jun. 5, 2003

(54) Automatic injector

(75) Inventors: Gilad Lavi, Lezion, IL (US); Gil Yigal, Gan-Yavne, IL (US); Izrail Tsals, Newtown, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: ELAN PHARMA INTERNATIONAL LIMITED WIL HOUSE, SHANNON (IE)

(21) Appl. No.: 10/290,664

(22) Filed: Nov. 8, 2002

Related U.S. Application Data
(60) Provisional application No. 60/334,294, filed on Nov. 30, 2001.

(51) Int. Cl. A61M 5/20

(52) U.S. Cl. 604/136; 604/890.1

(57) ABSTRACT

An injector is automatic in that the needle is injected automatically into the injection site (e.g., a patient's skin), delivery is initiated upon activation of the injector, and the needle is retracted after the end of delivery. Preferably the needle is not seen by the user prior to, during or after injection. Prior and to and after injection, the needle is withdrawn into the device so as to avoid any potential injury or health risk to the user or health care provider. The injector includes a housing and a control unit arranged to slide within the housing to move a piston rod during drug delivery and to pivot within the housing for needle retraction. The injector may also include a back rod that moves the piston rod before activation of the injector for titration and reconstitution and automatically disengages from the piston rod upon activation of the injector. A needle-locking device can be used in any number of pen-like injectors or other types of injectors or syringes. The needle-locking device includes a clip that insures that a needle assembly within an injector is in a locked position before and after use.

### RELATED APPLICATIONS

This is a utility application based upon provisional application serial No. 60/334,294, filed on Nov. 30, 2001, entitled AUTOMATIC INJECTOR. Applicants hereby claim the benefit of the filing date of the '294 provisional application for this utility application.

### FIELD OF THE INVENTION

This invention relates to the preparation and administration of a product into a living organism (e.g. the human body), and more particularly to an apparatus for automatically and safely delivering the product.

### BACKGROUND OF THE INVENTION

Previously, various devices have been developed for the delivery of medications into and through the skin of living organisms. These devices include syringes in which a liquid drug solution is delivered through the skin of a user from a syringe chamber by movement of a syringe plunger to move the drug solution from a syringe chamber through a syringe needle inserted under the skin. The drug solution is generally in liquid form, and can be a mixture of the drug (e.g. powdered, lyophilized, concentrated liquid) and a diluent (e.g. dextrox solution, saline solution, water).

It is well known that many persons are apprehensive of receiving an injection from a needle. This problem is even more significant for those who must administer their own medication. It is known that needle phobia can be minimized by hiding the needle before, during and after delivery. It is therefore preferable that the person who receives the drug should not see the needle, which often triggers the fear of needle injection.

It is also preferable for the needle to be protected before and after delivery of the drug. While a needle can be protected with a removable cap, it is preferable for the needle to be secured within the delivery device before the needle is injected through the patient's skin and after the needle is retracted. Preferably the needle is retracted back into the device and locked into final position after injection. The injection and retraction should be automatic, so that the user does not prematurely expose the needle for injection or have to guess when delivery is completed.

A user or patient could be injured if an injection device were activated prematurely. Generally, such a device projects its needle from the end of a barrel and ejects the dose. Such actions can cause injury if the needle pierces another person or is injected into an undesired area of the patient (e.g. an eye). Accordingly, it is advantageous if the needle is locked in a safe location before and after use to prevent accidental injury or contamination.

It is further desirable to have a simple, reliable system that facilitates safe preparation and delivery of a drug. Dosage amounts may vary from one patient to another. At present there is no easy way for a patient to self-administer a dosage of drug via an automatic injection system where the dosage amount may be easily measured prior to delivery and easily delivered. Moreover, there is a need to further improve the ability for the user to minimize residual drug in the container or system. Also, there is a need to enable the user to eliminate any air bubbles that may be trapped in the automatic system prior to use. There is also a need to combine the ability to titrate a dosage with the ability to reconstitute or lyophilize solid drug into a liquid form and to quickly, easily and safely administer the drug. The ability to combine these features is not available at the present time.

It is also desirable to provide a delivery system where the dosage for delivery is easily viewed by the patient prior, during and after use. At present, there is no known automatic delivery system that provides for the ability of the user to see the dosage formulation prior to use. The user's inability to see the dosage form prior to use creates a significant sense of unease in the user in that the user wants to ensure that the proper dosage is in the system and ready for delivery. More importantly, the user's inability to see the dosage form prior to use leaves the user concerned that the dosage may have air bubbles trapped therein and if present, may result in injury or harm or death to the user. The user's inability to view the dosage form being delivered and the end of delivery leaves the user with a level of uncertainty as to the amount delivered and the delivery being completed. Thus, it is extremely important to the user's peace of mind to provide an area in which to view the dosage prior to, during and after delivery.

Further, it is desirable to provide a delivery system that is easy to use at a low cost. Moreover, it is desirable to provide a system that is easy to integrate with the drug providing flexibility in meeting different drug containers like pre-filled syringes/cartridges, and empty syringes to be filled by the user.

### SUMMARY OF THE INVENTION

In an exemplary embodiment, an automatic injector for delivering a fluid includes a housing, a syringe, a control unit, and a driving unit. The housing has a proximate end and a distal end, and includes an activator trigger arranged to activate the injector. The syringe is positioned within the housing and includes a barrel, a piston rod, and a needle extending toward the distal end of the housing. The barrel is arranged to contain a fluid in communication with the needle. The piston rod is slidingly located within the barrel for forcing the fluid through the needle upon activation of the injector. The control unit is adapted to engage the piston rod and to slide and pivot within the housing. The driving unit is in communication with the housing and the control unit. The driving unit is arranged to bias the control unit, causing the control unit to slide towards the distal end and pull the piston rod through the barrel to force the fluid through the needle for delivery into an injection site. The control unit pivots out of engagement with the piston rod at the end of delivery.

The syringe may have a longitudinal axis different than a longitudinal axis of the driving unit. The injector may also include an automatic retracting mechanism that automatically retracts the needle into the housing upon an end of delivery. In addition, this exemplary embodiment of an injector may also include a needle-locking device that locks the needle within the housing before and after use. Further, this exemplary embodiment may include a back rod arranged for moving the piston rod for titration and/or reconstitution before delivery. Moreover, this exemplary embodiment may include a window that allows a user to inspect the dosage before delivery.

In another exemplary embodiment of the invention, an automatic injector for delivering a fluid includes a housing, a syringe, control means and driving means. The housing includes means for activating the injector, the housing having a proximate end and a distal end.. The syringe is positioned within the housing and includes a needle extending towards the distal end, a barrel, and a piston rod. The barrel is arranged to contain fluid therein in communication with the needle. The piston rod is slidingly located within the barrel for forcing the fluid through the needle upon activation of the injector. The control means is for engaging the piston rod during delivery and for pivoting out of engagement with the piston rod at the end of delivery. The driving means is for pulling the piston rod through the barrel, via the control means, to move the fluid through the needle for delivery into an injection site.

The means for activating preferably includes a trigger or an actuator. The control means preferably includes a control unit. The driving means preferably includes a driving unit (e.g., a spring, a gas chamber).

In still another exemplary embodiment, an automatic injector for titration and delivery of a fluid includes a housing, a syringe, a displacer and a driving unit. The housing includes an activator arranged to activate the injector, said housing having a proximate end and a distal end. The syringe is positioned within said housing. The syringe has a needle extending towards said distal end, a barrel, and a piston rod. The barrel is arranged to contain the fluid therein, the fluid in communication with said needle. The piston rod is slidingly located within said barrel for forcing the fluid through said needle upon activation of said injector. The displacer is arranged to communicate with said piston rod before the activation. The displacer is arranged to move said piston rod for titration before the activation and to automatically separate from said piston rod after titration. The driving unit is in communication with said housing and in communication with said piston rod. The driving unit is arranged to move said piston rod through said barrel upon the activation to push the fluid through said needle for delivery into an injection site.

In still another exemplary embodiment, an automatic injector for titration and delivery of a fluid includes a housing, a syringe, titration means and driving means. The housing includes means for activating said injector. The housing has a proximate end and a distal end. The syringe is positioned within said housing. The syringe has a needle extending towards said distal end, a barrel, and a piston rod. The barrel is arranged to contain the fluid therein, the fluid in communication with said needle. The piston rod is slidingly located within said barrel for forcing the fluid through said needle upon activation of said injector. The titration means is for moving said piston rod into said barrel before the activation to expel undesired air or fluid from the syringe and for automatically separating from said piston rod after the activation. The driving means is for moving said piston rod through said barrel upon the activation to push the fluid through said needle for delivery into an injection site.

In yet another exemplary embodiment, a needle-locking device prevents exposure of a needle held by a needle hub. The needle-locking device includes a clip having a hollow sleeve, a first set of latches and a second set of latches. The hollow sleeve supports the needle hub, having a first rim towards a proximate end of the needle hub and a second rim towards a distal end of the needle hub. The sleeve is adapted to slide longitudinally about the needle hub. The first set of latches extends longitudinally from the first rim, and is adapted to prevent axial movement of the needle hub after a delivery of a fluid from the needle. The second set of latches extends longitudinally from the second rim, and is adapted to prevent axial movement of the needle hub before and after delivery of the fluid.

The needle-locking device preferably includes a cartridge shell housing the clip. The shell includes grooves formed in its inner circumferential wall aligned with the second set of latches to accept the latches in a sliding relationship. In one example of this exemplary embodiment, at least one of the grooves includes an aperture extending radially outward through the shell for holding one of the second set of latches and preventing axial movement of the clip and of the needle hub prior to delivery. In another example of this exemplary embodiment, one of the grooves includes a locking slit extending radially outward from the inner circumferential wall for holding one of the second set of latches and preventing axial movement of the clip after delivery.

Further scope of applicability of the present invention will become apparent in the description given hereafter. However, it should be understood that the detailed description and specific examples, while indicating preferred embodiments of the invention, are given by way of illustration only, since the invention will become apparent to those skilled in the art from this detailed description.

### BRIEF DESCRIPTION OF THE DRAWINGS

The invention will be described in conjunction with the following drawings, in which like-referenced numerals designate like elements, and wherein:

FIG. 1 is a longitudinal section view showing an injector construed in accordance with an exemplary embodiment of the invention;

FIG. 2 is a view similar to that of FIG. 1, but showing the injector in a state wherein the protective cap is removed;

FIG. 3 is a partial sectional view of the injector illustrating FIG. 1, but showing the injector in a state wherein the trigger is depressed to activate the injector;

FIG. 4 is a view similar to that of FIG. 1, but showing the injector in a state during drug delivery;

FIG. 5 is a view similar to FIG. 1, but showing the injector at the end of delivery;

FIG. 6 is view similar to FIG. 5, but showing the control unit disengaged from the piston rod;

FIG. 7 is a view similar to FIG. 1, but showing the injector in a retracted state;

FIG. 8 is a longitudinal sectional view showing the disposable cartridge of a reusable injector in accordance with another exemplary embodiment of the invention;

FIG. 9 is a view similar to FIG. 8, but showing the vial attached to the cartridge;

FIG. 10 is a view similar to that of FIG. 8, but showing the cartridge in a state wherein a rod extends through a barrel;

FIG. 11 is a view similar to that of FIG. 8, but showing the drug aspirated into the syringe;

FIG. 12 is an isometric view of the syringe adapter and vial shown in FIG. 9;

FIG. 13 is a partial sectional view of the cartridge adapter and vial shown in FIG. 9;

FIG. 14 is an isometric of a locking clip for use with exemplary embodiments of the invention;

FIG. 15 is a longitudinal sectional view of the cartridge shown in FIG. 9 partially inserted into a reusable injector constructed in accordance with another exemplary embodiment of the invention;

FIG. 16 is a view similar to that of FIG. 15, but showing the cartridge fully loaded into the injector and the adapter and vial removed;

FIG. 17 is a longitudinal sectional view similar to that of FIG. 16, but showing the injector at end of delivery;

FIG. 18 is a longitudinal sectional view similar to FIG. 17, but showing the injector in its retracted position;

FIG. 19 is a partial sectional view showing the exemplary needle-locking mechanism;

FIG. 20 is a view similar to that of FIG. 19, but showing the cartridge fully loaded in the injector;

FIG. 21 is a view similar to that of FIG. 20, but showing the needle-locking mechanism during drug delivery;

FIG. 22 is a view similar to that of FIG. 21, but showing the needle-locking mechanism after delivery in its retracted position;

FIG. 23 is a longitudinal sectional view of an injector in accordance with another exemplary embodiment of the invention having a leaf lock;

FIG. 24 is a longitudinal sectional view similar to that of FIG. 23, but showing the injector during activation;

FIG. 25 is a longitudinal sectional view similar to that of FIG. 24, but showing the driving unit engaging the piston rod;

FIG. 26 is a longitudinal sectional view similar to that of FIG. 25, but showing the injector during drug delivery;

FIG. 27 is a longitudinal sectional view similar to that of FIG. 26, but showing the injector at the end of delivery;

FIG. 28 is a longitudinal sectional view similar to that of FIG. 27, but showing the control unit pivot to disengage with the piston rod;

FIG. 29 is a longitudinal sectional view similar to that of FIG. 28, but showing the injector in its locked retracted state;

FIG. 30 is a longitudinal sectional view showing an injector construed in accordance with another exemplary embodiment of the invention;

FIG. 31 is a longitudinal sectional view showing an injector construed in accordance with another exemplary embodiment of the invention;

FIG. 32 is a view similar to that of FIG. 31, but showing the injector in a state ready for use;

FIG. 33 is a longitudinal sectional view similar to that of FIG. 32, but showing the injector in its activating state;

FIG. 34 is a longitudinal sectional view similar to that of FIG. 33, but showing the injector after its activation;

FIG. 35 is a longitudinal sectional view similar to that of FIG. 34, but showing the injector during drug delivery;

FIG. 36 is a longitudinal sectional view similar to that of FIG. 35, but showing the injector at end of delivery;

FIG. 37 is a longitudinal sectional view showing an injector constructed in accordance with another exemplary embodiment of the invention;

FIG. 38 is a longitudinal sectional view similar to that of FIG. 37, but showing the injector ready for use;

FIG. 39 is a side view of the injector shown in FIG. 38, but showing the injector after activation;

FIG. 40 is a isometric view of the injector of this exemplary embodiment before activation;

FIG. 41 is a partial isometric view similar to FIG. 40, but showing the injector from a different perspective;

FIG. 42 is a partial isometric view similar to that of FIG. 40, but showing the injector upon activation;

FIG. 43 is a partial isometric view similar to that of FIG. 41, but showing the injector upon activation;

FIG. 44 is a longitudinal sectional view similar to that of FIG. 72, but showing the injector during drug delivery;

FIG. 45 is a longitudinal sectional view similar to that of FIG. 44, but showing the injector in a state after the end of delivery;

FIG. 46 is a longitudinal sectional view similar to that of FIG. 45, but showing the injector in its retracted state;

FIG. 47 is a longitudinal sectional view similar to that of FIG. 46, but showing the injector removed from the injection site;

FIG. 48 is an isometric view showing an injector constructed in accordance with another exemplary embodiment of the invention;

FIG. 49 is a longitudinal sectional view of the injector shown in FIG. 48;

FIG. 50 is a longitudinal sectional view similar to that of FIG. 49, but showing the injector with a dual chamber cartridge enclosed;

FIG. 51 is a longitudinal sectional view similar to that of FIG. 50, but showing the injector after the needle is connected to the cartridge;

FIG. 52 is a longitudinal sectional view similar to FIG. 51, but showing the injector prior to reconstitution;

FIG. 53 is a longitudinal sectional view similar to that of FIG. 52, but showing the injector after reconstitution;

FIG. 54 is a longitudinal sectional view similar to that of FIG. 53, but showing the injector in an activated state;

FIG. 55 is a longitudinal sectional view similar to that of FIG. 54, but showing the injector during drug delivery;

FIG. 56 is a longitudinal sectional view similar to that of FIG. 55, but showing the injector after retraction;

FIG. 57 is a longitudinal sectional view similar to that of FIG. 50, but showing the injector with a single chamber cartridge;

FIG. 58 is top view showing an injector constructed in accordance with another exemplary embodiment of the invention;

FIG. 59 is a longitudinal sectional view similar taken along line A-A of FIG. 58;

FIG. 60 is a partial sectional view taken along line B-B of FIG. 58;

FIG. 61 is a longitudinal sectional view similar to that of FIG. 59, but showing the injector in a state after titration;

FIG. 62 is a partial sectional view similar to that of FIG. 60, but showing the syringe in the state shown in FIG. 61;

FIG. 63 is a longitudinal sectional view similar to that of FIG. 61, but showing the injector after activation;

FIG. 64 is a partial sectional view similar to that of FIG. 62, but showing the injector in the state shown in FIG. 63;

FIG. 65 is a longitudinal sectional view similar to that of FIG. 63, but showing the injector after needle penetration of the tissue;

FIG. 66 is a partial sectional view similar to that of FIG. 64, but showing the injector in the state shown in FIG. 65;

FIG. 67 is a longitudinal sectional view similar to that of FIG. 65, but showing the injector's control unit rotated and detached from the syringe;

FIG. 68 is a longitudinal sectional view similar to that of FIG. 67, but showing the injector during drug delivery;

FIG. 69 is a longitudinal sectional view similar to that of FIG. 68, but showing the injector at end of delivery;

FIG. 70 is a longitudinal sectional view similar to that of FIG. 69, but showing the injector after end of delivery;

FIG. 71 is a longitudinal sectional view similar to that of FIG. 78, but showing the injector after retraction;

FIG. 72 is a longitudinal sectional view similar to that of FIG. 71, but showing the injector removed from the injection site;

FIG. 73a is an expanded isometric view of the elements featured in FIGS. 59-72;

FIG. 73b is an exploded isometric sectional view of the elements shown in FIG. 73a;

FIG. 74a is a side view corresponding to the injector shown in FIG. 59;

FIG. 74b is a side view corresponding to the injector shown in FIG. 61;

FIG. 74c is a side view corresponding to the injector shown in FIG. 65;

FIG. 74d is a side view corresponding to the injector shown in FIG. 71;

FIG. 75 is a partial sectional view showing the injector in a pre-activation state;

FIG. 76 is a partial sectional view similar to FIG. 75, but showing the injector in another pre-activation state;

FIG. 77 is a partial sectional view similar to that of FIG. 76, but showing the injector pressed against an injection site;

FIG. 78 is a partial sectional view similar to that of FIG. 77, but showing the injector at an activation state;

FIG. 79 is a longitudinal sectional view showing in injector constructed in accordance with another exemplary embodiment of the invention;

FIG. 80 is a longitudinal sectional view similar to that of FIG. 79, but showing the injector during activation;

FIG. 81 is a longitudinal sectional view similar to that of FIG. 80, but showing the injector during needle injection;

FIG. 82 is a longitudinal sectional view similar to that of FIG. 81, but showing the injector during drug delivery;

FIG. 83 is a longitudinal sectional view similar to that of FIG. 82, but showing the injector just before end of delivery;

FIG. 84 is a longitudinal sectional view similar to that of FIG. 83, but showing the injector at end of delivery;

FIG. 85 is a longitudinal sectional view similar to that of FIG. 84, but showing the injector after end of delivery and before needle retraction;

FIG. 86 is a longitudinal sectional view similar to that of FIG. 85, but showing the injector after needle retraction; and

FIG. 87 is an enlarged sectional view taken along line 87-87 of FIG. 79.

### DETAILED DESCRIPTION OF THE INVENTION

The present invention is directed to automatic injectors and needle-locking devices.. The injector is automatic in that the needle is automatically extracted out of a distal end of the injector and injected into the injection site (e.g., a patient's skin), delivery is initiated upon activation of the injector, and the needle is automatically retracted after the end of delivery. The exemplary injectors include a control unit that both slides and pivots in the injector. Moreover, the exemplary injectors include a displacer or back rod that provides titration and reconstitution as described below.

The term distal refers to the end or direction of the injector that is applied to an injection site for delivery. The term proximate refers to the end of the injector that is opposite the distal end. The exemplary embodiments show each injector having a distal end from which the needle is exposed for delivery, and a proximate end opposite the distal end.

Preferably the needle is not seen by the user prior to, during or after injection. Prior to and after injection, the needle is withdrawn into the injector so as to avoid any potential injury or health risk to the user or health care provider.

Without being limited to any particular theory, the needle-locking device can be used in any number of pen-like injectors or other types of injectors or syringes. The needle-locking device includes a clip that insures that a needle assembly within an injector is in a locked position before and after use. For purpose of illustration, the needle-locking device is shown in combination with a drug cartridge arranged for insertion in a housing of the injector. Of course, the needle-locking device can also be used in a housing without the cartridge. Preferably this housing would include a sleeve or other structure beneficial for operation of the clip.

Without being limited to a particular theory, the disclosed exemplary embodiments include: a) a reusable device having a disposable cartridge with pre-filled syringe; b) a disposable cartridge with locking device for reusable devices; c) a pre-filled fully disposable injector with titration and syringe lock; d) an empty fully disposable injector with mixing, titration and reconstitution; e) an empty fully disposable injector with mixing, titration, reconstitution, syringe lock and body sensor; f) a disposable pre-filled, dual chamber cartridge for lyophilized drug; g) a disposable pre-filled, single chamber liquid drug cartridge; h) a fully disposable pre-filled injector with drug titration (needle concentric to housing); i) a fully disposable pre-filled injector with drug titration and improved trigger assembly; and j) a concentric disposable injector with drug titration.

### Reusable Device, Disposable Cartridge with Pre-Filled Syringe

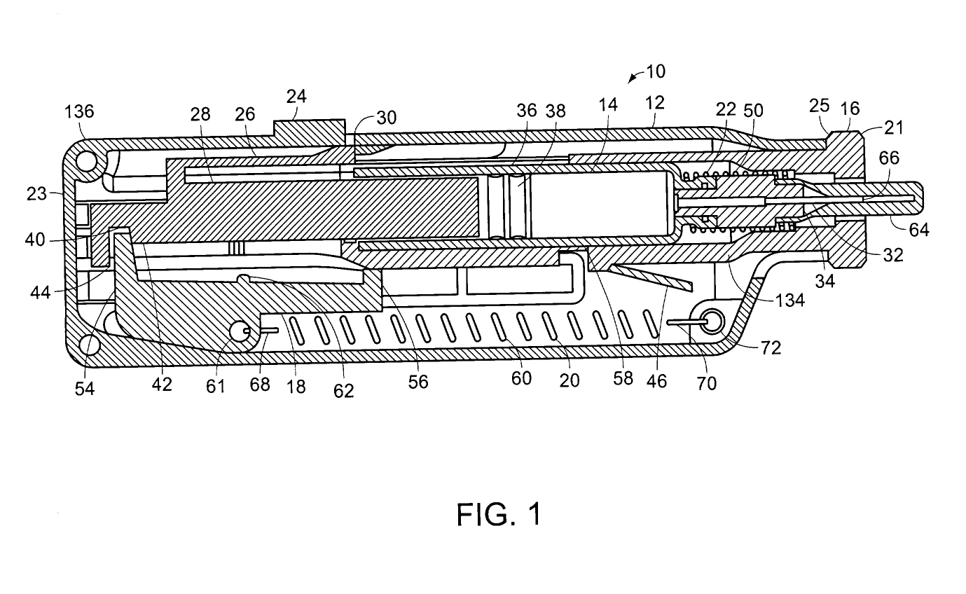

Referring to FIG. 1, an automatic injector is schematically illustrated at 10. The injector 10 includes a housing 12, a syringe 14, a cartridge 16, a control unit 18, a driving unit 20, and a retracting unit 22. The injector 10 has a distal end 21 and a proximate end 23. While the figures illustrating this exemplary embodiment shown the injector 10 as reusable and having a disposable cartridge 16 with a pre-filled syringe 14, it is understood that this embodiment is applicable to both reusable and disposable injectors with empty or pre-filled syringes 14. The structural elements described in this exemplary embodiment can by formed of any suitable material, e.g. plastic, metal, rubber.

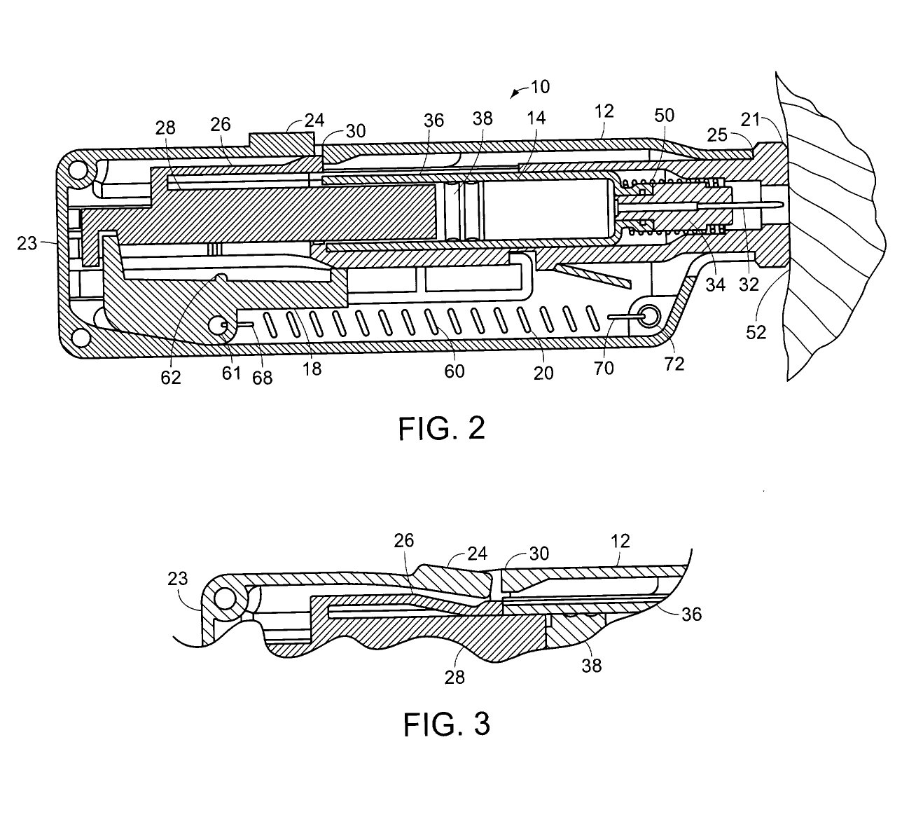

Referring to FIGS. 1-7 the housing 12 includes a trigger 24 and an opening 25. The trigger 24 is arranged to activate the injector 10, which will be described in more detail below. While a trigger has been selected as an actuator or as the means for activating the injector 10, it is understood by those skilled in the art that any other alternative means of activation including but not limited to spring-loaded, electronic, sliding, rotating, magnetically operated mechanisms could be used to achieve the same result. The trigger 24 is shown adjacent a holding latch 26 in FIGS. 1-3 that extends from a piston rod 28. As can best be seen in FIGS. 1 and 2, the holding latch 26 abuts a shoulder 30 of the housing 12 prior to activation of the injector 10. The holding latch 26 ensures needle penetration prior to the liquid delivery, as will be described in greater detail below. The opening 25 is located at the distal end 21 for receiving the cartridge 16.

The syringe 14 shown in FIGS. 1-7 includes the piston rod 28, a needle 32, a needle hub 34, and a barrel 36 arranged to contain a fluid drug in communication with the needle 32. The piston rod 28 is slidingly located within the barrel 36 and is shown extending from the barrel 36 toward the proximate end 23 of the injector 10. The holding latch 26 is shown in FIG. 1 abutting the shoulder 30 to temporarily prevent the piston rod 28 from moving toward the distal end 31 of the injector 10. The piston rod 28 is designed to force the fluid through the needle 32 upon activation of the injector 10, which will be discussed in more detail below. A plug 38 is shown in the barrel 36 attached to the piston rod 28 for sealing the drug in the barrel 36. The plug 38 may be considered as integral with the piston rod 28 or as part of the syringe 14 sealing the drug in the barrel 36.

The piston rod 28 includes a rear notch 40 having a rear facing shoulder 42 facing the proximate end 23 of the injector 10 that is engaged to urge the piston rod 28 through the barrel 36 of the syringe 14. The piston rod 28 shown in FIGS. 1-7 also includes a front-facing shoulder 44 offset from and facing the rear-facing shoulder 42. The front-facing shoulder 44 is engaged by the control unit 18 for pulling the piston rod 28 from the syringe 14 when the cartridge 16 is loaded into the housing 12.

The syringe 14 shown in FIGS. 1-7 is prefilled with a drug for injection. However, the syringe 14 may be empty and filled by the user before use as will be described in greater detail below. Also in FIGS. 1-7, the syringe 14 is slidingly located within a cartridge 16. Preferably the syringe 14 is loaded into the cartridge 16, and the cartridge 16 is loaded into the housing 12 by the user.

The cartridge 16 is locked in the housing 12 by a cartridge latch 46. As shown in FIG. 1, the cartridge latch 46 is attached to the housing 12 and abuts the cartridge 16 to prevent forward axial movement of the cartridge 16 during injection. This cartridge 16 enables the injector 10 to be reused, as the cartridge 16 can be discarded after each use. The cartridge 16 can be discarded, for example, by moving the cartridge latch 46 away from the cartridge 16, and pulling the cartridge 16 out of the opening 25 of the housing 12.

The needle hub 34 attaches to both the needle 32 and the barrel 36 for providing fluid communication there between. A retracting unit 22 is shown in FIG. 1 between the cartridge 16 and the needle hub 34. Preferably, the retracting unit 22 is a spring 50 under compression arranged to push the syringe 14 away from an injection site 52 (see FIG. 2), thereby retracting the needle 32 from the injection site 52 into the housing 12 when in use. The spring 50 automatically retracts the needle 32, as will be described in greater detail below. The needle 32 includes a sharp point 66 at its distal end for puncturing through any outer layer of the injection site 52 so that drug can be delivered into the injection site 52.

The control unit 18 includes an engaging finger 54, a disengaging finger 56, an aperture 61 and an axis 62. The engaging finger 54 abuts the rear facing shoulder 42 of the piston rod 28, as will be described in greater detail below. During injection, the engaging finger 54 extends behind the rear-facing shoulder 42 of the piston rod 28 to move the piston rod 28 into the barrel 36 of the syringe 14. The disengaging finger 56 of the control unit 18 extends radially inward and is adapted to slide along the cartridge longitudinal extended rails 134 of the disposable cartridge 16 into a disengaging notch 58 of the cartridge 16 at the end of delivery. This movement pulls the engaging finger 54 from its abutment against the rear facing shoulder 42 and disengages the control unit 18 from the piston rod 28, as described in greater detail below. The aperture 61 is a recess that connects with the driving unit 20. In this embodiment, the axis 62 is a projection that communicates with the cartridge 16 as a pivot point of the control unit 18.

While the control unit is selected as a control means for engaging the piston rod and pivoting out of engagement with the rod, it is understood that the control means is not limited to a control unit having the particular shapes shown in the exemplary embodiments, as long as the control unit is adapted to both slide and pivot within the housing as described herein, and as equivalents thereof The driving unit 20 is in communication with the housing 12 and the control unit 18 to bias the control unit 18 and pull the piston rod 28 through the barrel 36. The driving unit 20 is preferably a spring 60 having a first end 68 and a second end 70. As shown in FIGS. 1-7, the driving unit 20 is an extension spring attached between the control unit 18 and the housing 12. While a spring has been selected as the driving unit or as the driving means for moving the piston rod through the barrel, it us understood by those skilled in the art that any other alternative driving unit or driving means including but not limited to mechanical, electrical, or gas mechanisms similarly located in the housing could be used to achieve the same result.

As can be seen from FIG. 1, the driving unit 20 is attached to the control unit 18 by hooking the first end 68 of the spring into the aperture 61 within the control unit 18. The driving unit 20 is attached to the housing by hooking the second end 70 of the spring 60 into a recess 72 fixed to the housing 12. The recess 72 can also be configured as a hook or any other structure adapted to attach to the second end 70 of the spring 60, as readily understood by a skilled artisan. The driving unit 20 pulls the control unit 18 through the housing 12 toward the distal end 21 of the injector 10, thereby pulling the piston rod 28 through the syringe 14 and forcing the drug through the needle 32 in use.

At the end of delivery, the driving unit 20 continues to pull the control unit 18. Because the aperture 61 is located on a side of the axis 62, the control unit 18 slides through the housing 12 until it reaches the disengaging notch 58 at the end of delivery. When the control unit 18 reaches the notch 58, it pivots on its axis 62. This pivot moves the disengaging finger 56 into the disengaging notch 58 and moves the engaging finger 54 from the piston rod 28, thereby separating the driving unit 20 from the piston rod 28. The rotation or pivot of the control unit 18 is the result of the moment caused by the offset distance between the symmetry axis of the driving unit 20 and the axis 62 of the control unit 18.

As shown in FIG. 1, the injector 10 also includes a cap 64 preferably made of rubber or plastic that covers the sharp distal point 66 of the needle 32. The cap 64 extends out of the housing 12 to provide sterility protection for the needle 32. This protecting cap 64 is needed only for the pre-filled syringe.

FIGS. 1-7 generally illustrate the various steps for injection of the drug in accordance with a method of using the injector 10. To that end, the cartridge 16 is inserted into the housing 12. The cartridge 16 is inserted into the housing 12 by sliding the cartridge 16 though the opening 25 of the housing 12. The cartridge 16 should be aligned in the housing 12 during insertion so that the barrel 36 slides over the piston rod 28. The piston rod 28 does not have to reach the plug 38 during insertion. The piston rod 28 contacts the plug 38 only when the cartridge 16 is filled to its maximum volume. When the cartridge 16 is fully inserted into the housing 12, the cartridge latch 46 abuts the cartridge 16 and locks the cartridge 16 in place. As shown in FIG. 2, a user removes the cap 64 and places the distal end 60 of the housing 12, as shown in FIG. 1, at the injection site 52, as shown in FIG. 2. Preferably, the injector 10 is pressed against the injection site 52 to ensure that the injector 10 does not move during injection. The trigger 24 is then pressed into the housing as shown in FIG. 3 to activate the injector 10.

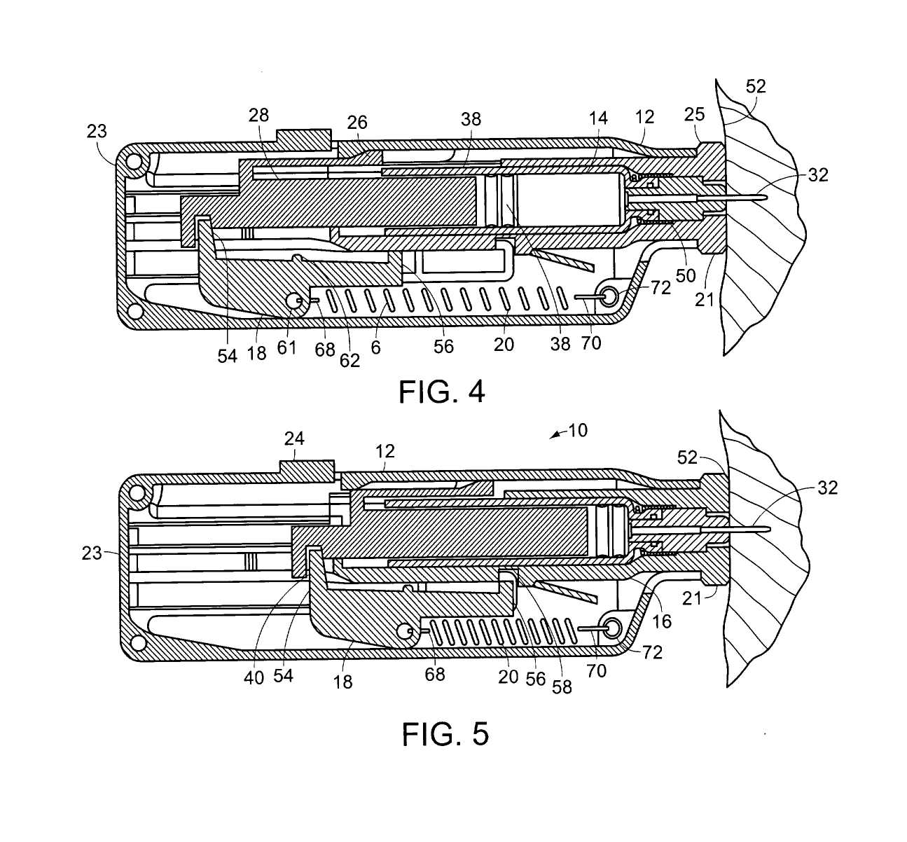

The depression of the trigger 24 releases the holding latch 26 from the housing 12 where it engages the syringe 14. This release causes the driving unit 20 (e.g., extension spring 60) to contract between the control unit 18 and the housing 12. This contraction of the driving unit 20 biases the control unit 18 causing the holding latch 26 now engaged with the syringe 14 to push the syringe 14 forward through the housing 12, causing the needle 32 to penetrate the tissue at the injection site 52. As shown in FIG. 4, when the syringe 14 is at its forward position, the holding latch 26 is biased radially outward toward the housing 12 and releases the syringe 14. However the driving unit 20 continues to pull the piston rod 28 forward, which pushes the drug through the needle 32 for delivery.

The end of delivery typically occurs when the driving unit 20 pulls the piston rod 28 through the barrel 36 to its forward limit. As shown in FIG. 5, even though the piston rod 28 is at its forward limit, the driving unit 20 continues to pull the control unit 18. This causes the control unit 18 to pivot about its projection or axis 62. As shown, the disengaging finger 56 of the control unit 18 becomes aligned with the disengaging notch 58 of the cartridge 16, permitting the control unit 18 to pivot and move the disengaging finger 56 into the disengaging notch 58. This pivot also pulls the engaging finger 54 of the control unit 18 away from its contact with the rear facing shoulder 42 of the piston rod 28, which releases the piston rod 28 from any bias of the driving unit 20. In other words, at the end of delivery, the control unit 18 pivots and moves the disengaging finger 56 into the disengaging notch 58, which locks the driving unit 20 in place. As a result, the retracting spring 50 is no longer under the force of the driving unit 20 and is able to push the syringe 14 and piston rod 28 backward from its previous direction of advancement, as shown in FIG. 7.

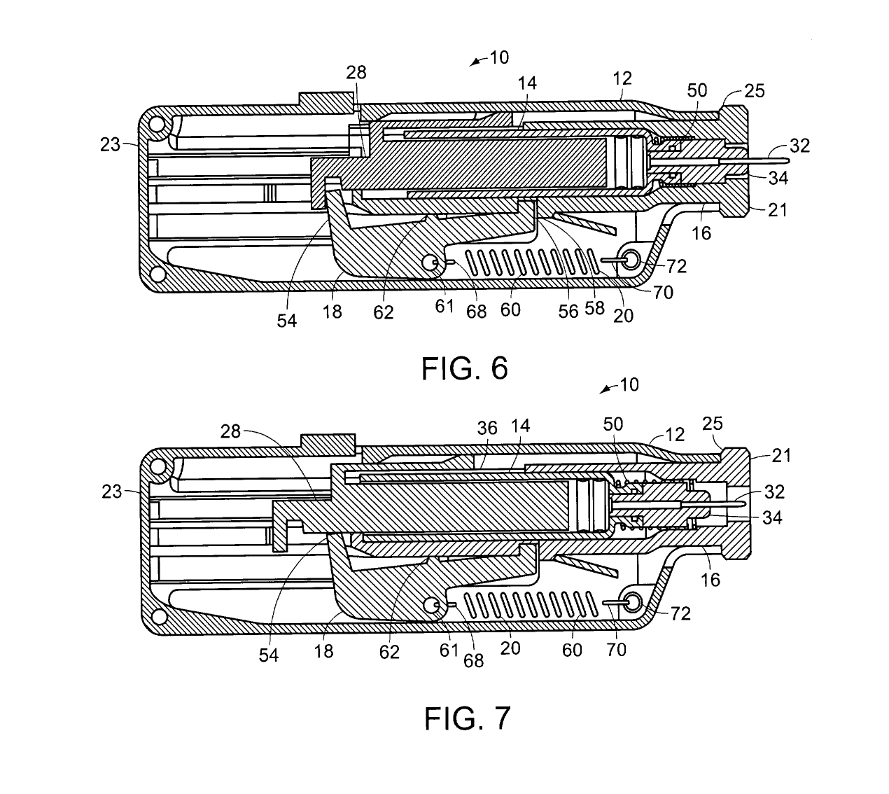

As shown in FIG. 6, after the pivot of the control unit 18, the piston rod 28 and the syringe 14 are not under the bias of the driving unit 20. The retracting spring 50, which preferably has a spring load less than the spring load of the driving unit 20, is free and able to push the syringe 14 back into the housing 12.

Once the retracting spring 50 reaches its original preload position, the length of the needle 32 is fully retracted into the housing 12. FIG. 7 shows the syringe 14 pushed back by the retracting spring 50. In its retracted position, the needle 32 is withdrawn from the injection site 52 back into the housing 12, and is safe for disposal.

To dispose the needle 32, the cartridge 16 is removed from the housing 12. The cartridge 16 is preferably removed by rotating the cartridge to disengage the cartridge latch 46 from the cartridge 16 and sliding the cartridge 16 out of the housing 12 through its opening 25, as readily understood by a skilled artisan.

### Disposable Cartridge With Locking Device For Reusable Devices Structure of Embodiment

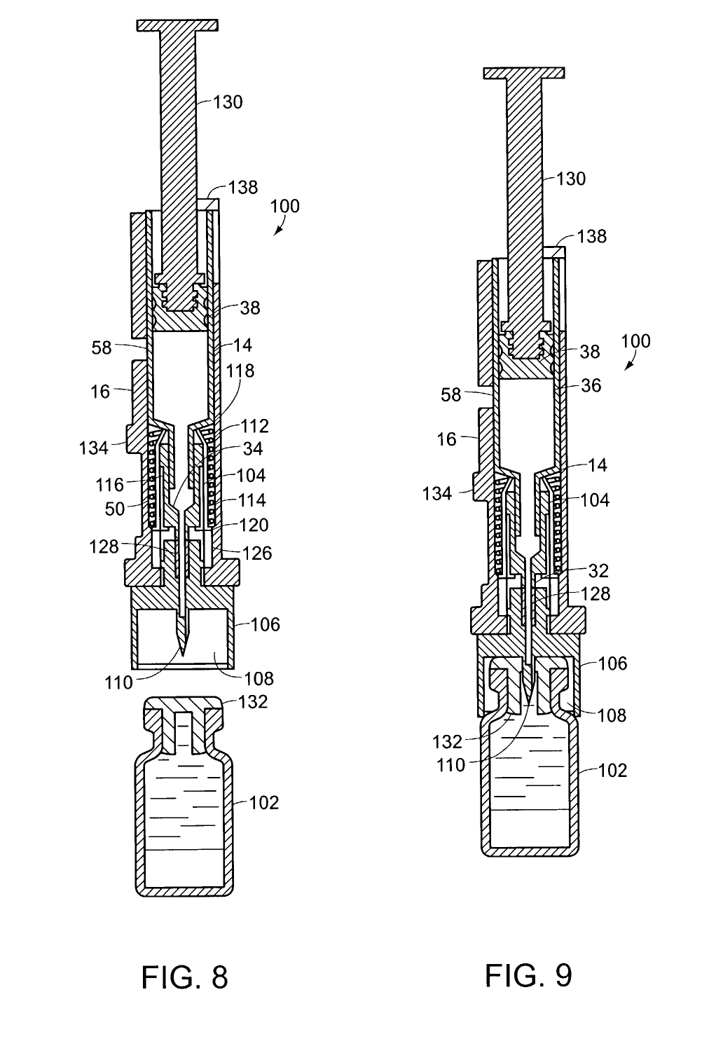

Referring to FIGS. 8-23, there is shown at 100 a cartridge assembly constructed in accordance with the reusable embodiments of this invention. The cartridge assembly 100 is disposable and can be used with the reusable injector 10 described above. The cartridge assembly may include a pre-filled syringe or an empty syringe to be filled by the user prior to injection. The description below relates to the use of empty syringe. The cartridge assembly 100 includes a cartridge 16, a syringe 14, a retracting spring 50, and a locking clip 104. Because the syringe 14 in the cartridge assembly 100 shown in FIG. 8 is empty, the cartridge assembly 100 also includes a cartridge rod 130. The cartridge rod 130 is attached to the plug 38 during the preparation process of filling the syringe 14. In this exemplary embodiment, the cartridge rod 130 is removed from the plug 38 after the syringe 14 is filled and before the cartridge 16 is loaded into the housing 12. The syringe 14 includes a barrel 36, a plug 38, a needle hub 34, and a needle 32.

The cartridge assembly 100 shown in FIG. 8 also includes an adapter 106 for coupling a drug vial 102 to the cartridge assembly 100. To that end, the adapter 106 includes a vial recess 108 and a spike 110. It is also within the scope of the invention that the adapter 106 might be just a guide and the syringe needle 32 is used to penetrate directly through a vial stopper 132 into the vial 102, as a skilled artisan would readily understand. The spike 110 may include an integrated metal needle or a plastic molded spike. The adapter 106 provides an approach for enabling, filling and reconstitution of a drug compound for delivery into an injection site 52.

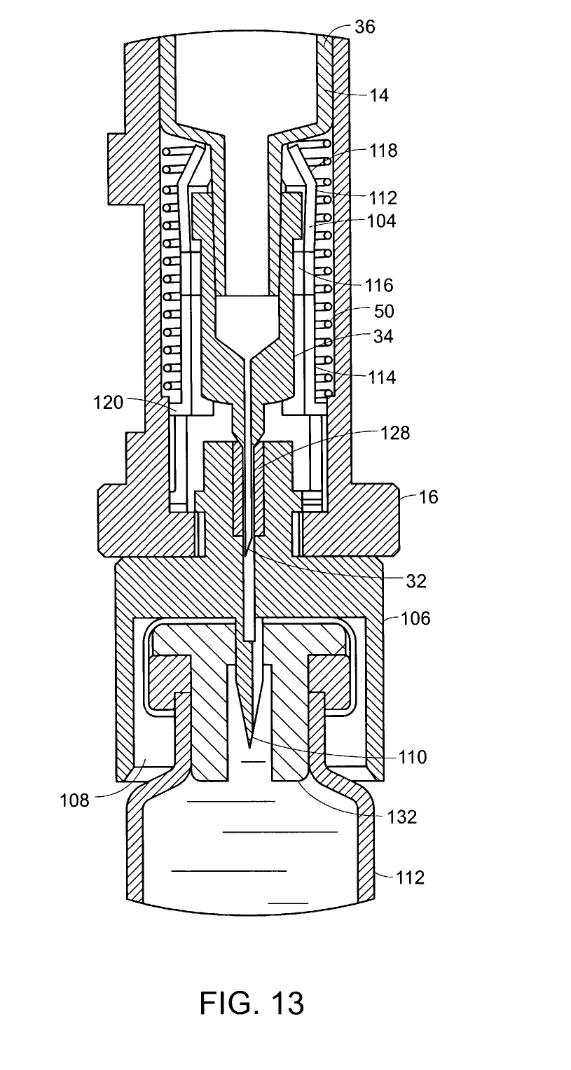

The cartridge 16, syringe 14 and retracting spring 50 have been described above and operate as described. As best seen in FIGS. 13, 14 and 19-22, the locking clip 104 is a multi-positioned ring having a set of fingers 112 and a set of latches 114. Preferably the clip 104 is semi-rigid formed of an elastic material, such as plastic. As shown in FIG. 14, all of the fingers 112 and latches 114 are connected to a common base 116, and each finger 112 and latch 114 operates like a leaf spring, trying to move to its original position when deflected. Each finger 112 extends longitudinally from the common base 116 in a first direction and is bent radially inward at its fingertips 118. These fingers 112 are arranged to block the needle 32 and needle hub 34 before and after use by the automatic injector 10.

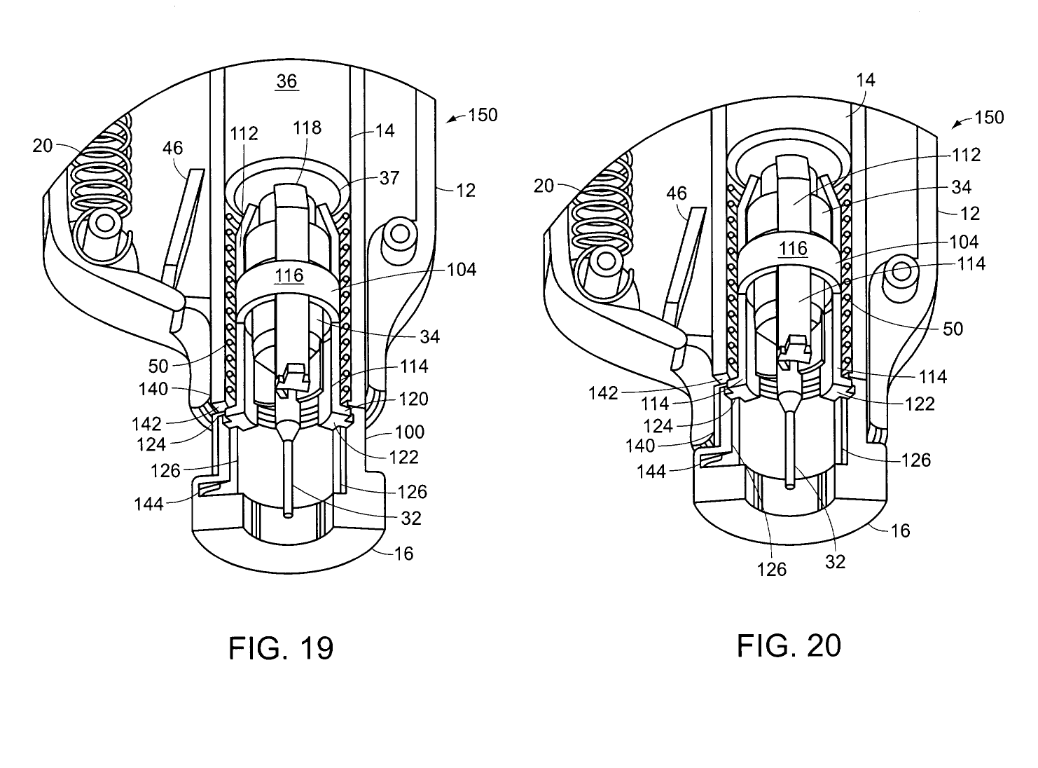

As can best be seen in FIG. 14, each latch 114 extends longitudinally opposite to the direction of extension of the fingers 112 to latch feet 120. At least one of the latch feet 120 extends radially outward and includes a wide section 122 and a narrow section 124. The narrow section 124 slides along a groove 126 (FIG. 19) in the cartridge 16 and locks the clip 104 in a first position. The wide section 122 prevents axial movement of the clip 104 before and after use. The locking clip 104 will be described in more detail below. However, it is understood that the locking clip 104 is applicable to various types of injectors, including the injector 10 described above and other exemplary injectors described throughout this application.

Referring to FIG. 13, the adapter 106 includes a sealing tube 128, preferably formed of an elastomeric material that seals the needle hub 34 to the adapter 106 and seals the path between the adapter 106 and the needle 32. Prior to inserting the vial 102 into the vial recess 108 of the adapter 106, the user should pull the piston rod 130 (FIG. 11) so the barrel 36 indicates the desired volume intended for aspiration. This desired volume preferably corresponds to the volume of drug intended for delivery.

### Filling the Cartridge With Solution

It is well known that air is not desired in a syringe during drug delivery, so holding the cartridge 16 upright with the needle 32 above the barrel during aspiration, titration and reconstitution is desired to keep air out of and remove air from the syringe. Without being limited to a particular theory, the exemplary procedure for filling the cartridge 16 with solution as related to the invention orients the vial 102 and cartridge 16 upright vertically (opposite of the orientation shown in FIGS. 8-11) so that the needle 32 is above the barrel 36. In this orientation, the liquid solution is aspirated from the vial 102 into the syringe 14, but air stays in the vial 102. This preferred orientation also provides the benefit of ensuring that any air in the syringe 14 is pushed out of the syringe 14 before the liquid solution during titration. In other words, the upright orientation keeps air away from the adapter spike 110 so that liquid is first aspirated into the syringe 14 and air is flushed out of the syringe 14. Accordingly, the amount of dosage can be accurately measured, and any air in the syringe 14 is removed so it can't be delivered into a patient.

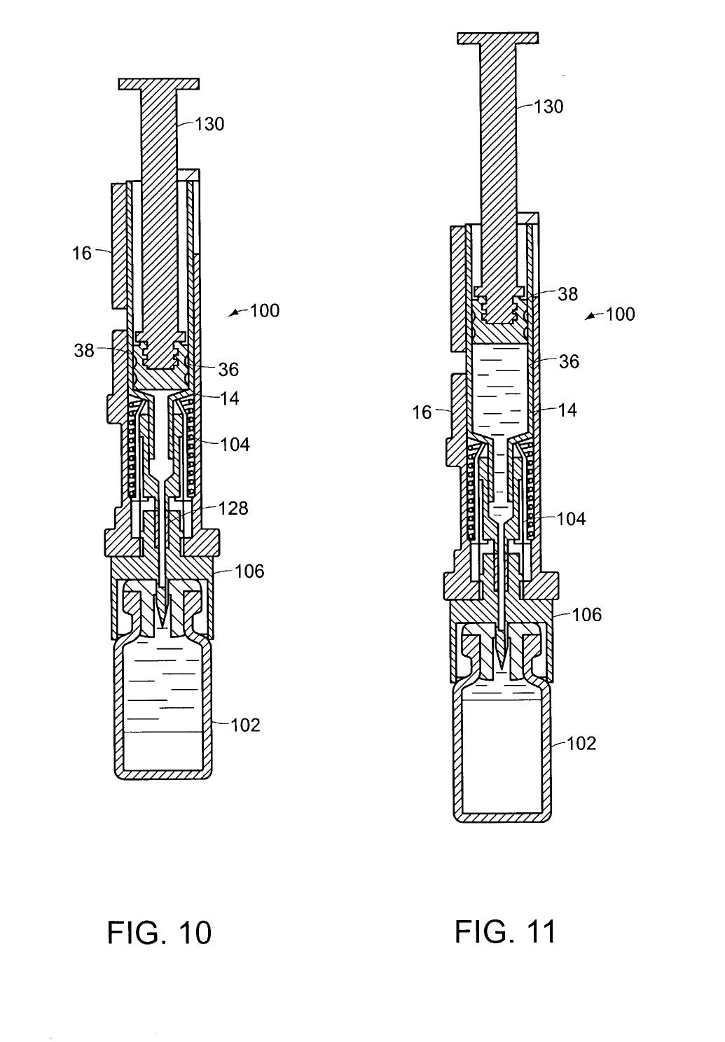

In FIG. 9, the vial 102 is inserted into the vial recess 108 of the adapter 106. The adapter spike 110 penetrates the vial stopper 132 to open the fluid path between the vial 102 and the syringe 14. As shown in FIG. 10, the piston rod 130 is depressed into the barrel 36 until air in the barrel 36 has been moved from the syringe 14 into the vial 102. In this state, the piston rod 130 and plug 38 are fully depressed into the barrel 36 of the syringe 14 to push the air into the vial 102. This action increases the pressure in the vial 102 and allows for easier retraction of the rod 130.

In FIG. 11, liquid drug solution in the vial 102 is aspirated into the syringe 14. The rod 130 and plug 38 are pulled back which causes the drug to move from the vial 102 into the syringe 14. In accordance with one exemplary embodiment, the syringe 14 is translucent and includes a series of visual indications scaled thereon, and the cartridge 16 is translucent, as understood by a skilled artisan, to enable an accurate measurement of the amount of liquid drawn into the barrel 36 of the syringe 14. Therefore, aspiration of the liquid is preferably measured by visual observation of the scale. If a user gets more liquid than desired, it is possible to push the excess fluid back into the vial 102 or even start the process again by pushing all of the fluid into the vial 102. Accordingly, a user can adjust the position of the plug 38 within the barrel 36 to the appropriate level of liquid.

As understood by a skilled artisan, the vial 102 can be replaced with another vial 102 containing a lyophilized drug or liquid drug for mixing with the liquid in the syringe 14. In this manner, several drug vials 102 can be used for reconstitution of a drug compound by replacing one drug vial 102 with another and mixing the contents of each drug vial 102 with the drug solution until the desired compound is mixed for injection. This operation should be carried out with the cartridge assembly 100 in a vertical position with the vial 102 inverted to prevent air from flowing into the syringe 14.

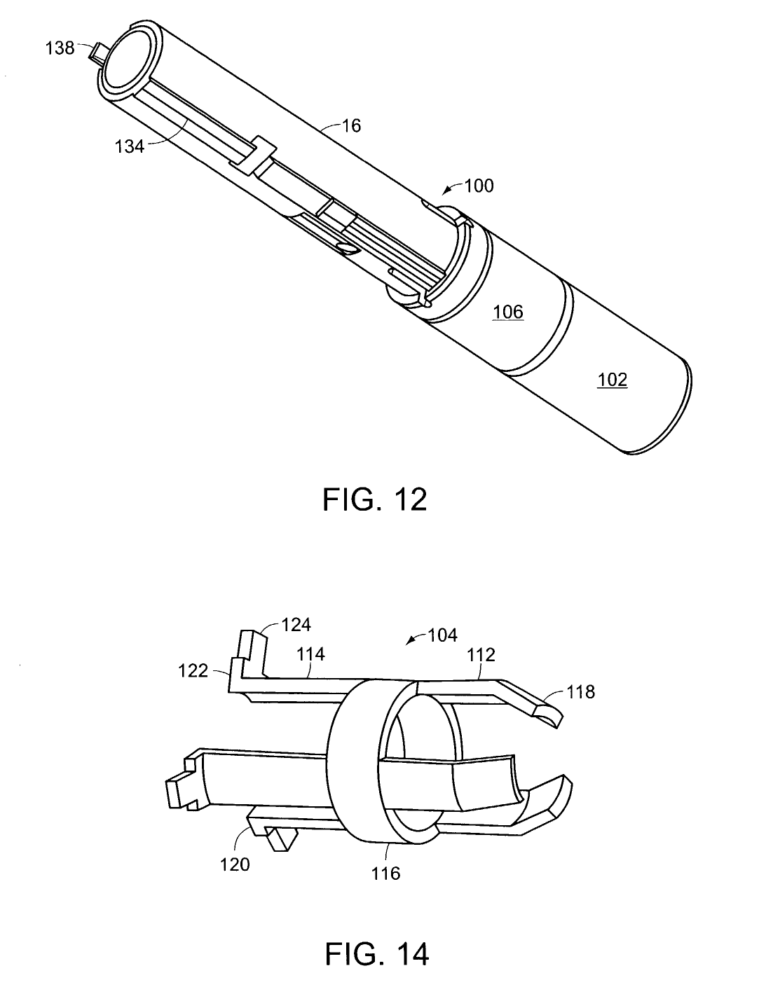

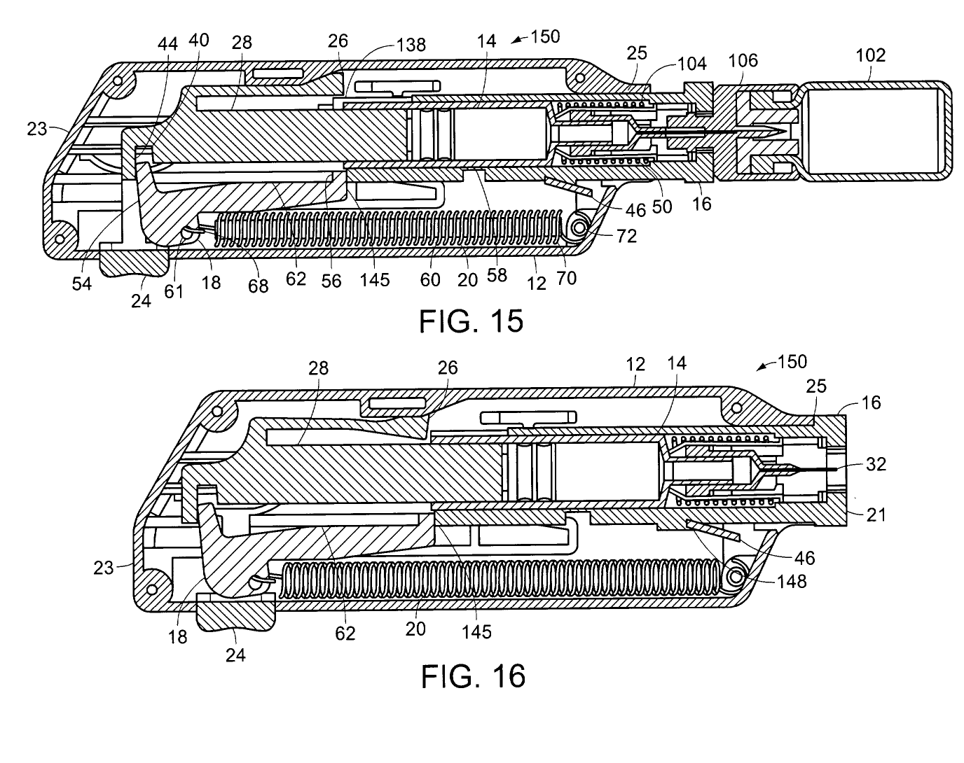

Once the syringe has the desired dose, the rod 130 is detached from the plug 38, and the cartridge assembly 100 is ready for loading into the injector 10. Preferably, the cartridge assembly 100 is loaded into the injector 10 by aligning the cartridge 16 with an opening at the distal end 25 of the housing 12 of the injector 10 and pushing the cartridge 16 into the injector 10 (FIG. 15). As shown in FIG. 12, the cartridge 16 preferably has rail 134 extending longitudinally along the outer wall of the cartridge 16. The rail 134 is stepped in a radial fashion for alignment and engagement with the cartridge latch 46. The rail is interrupted by the disengaging notch 58. As described in the above injector 10, the extended rail 134 is used to maintain the engagement of the control unit 18 with the piston rod 28, and the notch 58 for disengagement of the control unit 18 from the piston rod 28 at end of delivery.

FIG. 13 is a partial sectional view of the cartridge 16, syringe 14, locking clip 104, adapter 106 and vial 102 shown in FIGS. 8-11. As shown, the locking clip 104 is positioned between the retracting spring 50 and the needle hub 34 and is arranged to bias the syringe 14 away from the front face of the cartridge 16.

### Loading the Cartridge Into the Reusable Injector

FIGS. 15-22 illustrate the cartridge 16 described above with the injector 10. However, the injector 10 in these figures is slightly differs from the embodiment illustrated in FIGS. 1-7. For example, in FIGS. 15-22, the trigger 24 is located adjacent the control unit 18 instead of opposite the control unit 18, as shown in FIGS. 1-7. It is understood that the location of the trigger 24 depends on which element is desired for depression to activate the injector 10, and is not considered critical to the essence of the invention.

Referring to FIG. 15, as the cartridge 16 is inserted into the reusable injector 10, a rear wall 145 of the cartridge 16 abuts and pushes the control unit 18 backward towards the proximate end 23 of the injector 10. The engaging finger 54 of the control unit 18 abuts the front-facing shoulder 44 of the piston rod 28 and pushes the piston rod 28 rearward. While the control unit 18 is being pushed backward, it loads the driving unit 20. As mentioned earlier, the driving unit 20 may comprise an extension spring 60.

As can best be seen in FIG. 19, during insertion, the needle hub 34 is locked from movement in either longitudinal direction within the cartridge 16. The needle hub 34 is prevented from moving rearward towards the proximate end 23 of the injector 10 by the fingertips 118 of the fingers 112, and by a locking finger 138 at the back side of the cartridge 16 (FIGS. 12 and 15). The radially inward extending fingertips 118 also prevent forward movement since they abut a front wall 37 of the barrel 36. The latches 114 extend outwardly through grooves 126 in the cartridge 16 such that the wide section 122 blocks the grooves 126 in the cartridge 16, thereby preventing longitudinal movement of the clip 104.

FIGS. 16 and 20 show the cartridge 16 fully inserted into the injector 10. When using the cartridge as pre-filled cartridge, the syringe needle 32 is provided with a protective cover (FIG. 1) for providing drug sterility. The protective cover is preferably removed after the cartridge 16 is loaded into the injector 10.

The control unit 18 pushed by the rail 134 (FIG. 12) slides adjacent the trigger 24 and keeps the trigger 24 in its preactivation position. The cartridge 16 is locked in place by the cartridge latch 46 abutting a stepped shoulder 148 (FIG. 16) of the rail 134. As shown at FIGS. 16 and 20, when the cartridge 16 is inserted and locked, latches 114 are pushed radially inward. The radial push is caused by contact between the narrow section 124 of the latches 114 and inner circumferential walls 140 near the opening at the distal end of the housing 12. Once the latches 114 are pushed inwardly, their wide section 122 is no longer in the slot 142 and the narrow cross-section 124 of each latch 114 is free to move in and along the grooves 126 as the needle 32 moves forward.

### Locking the Needle After Injection

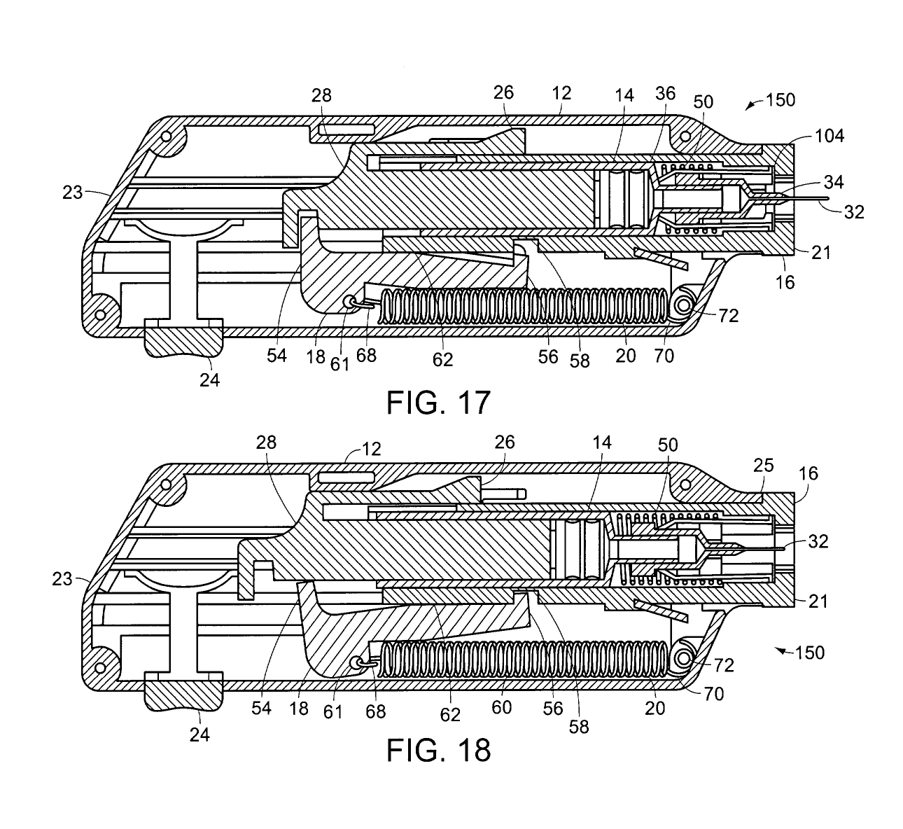

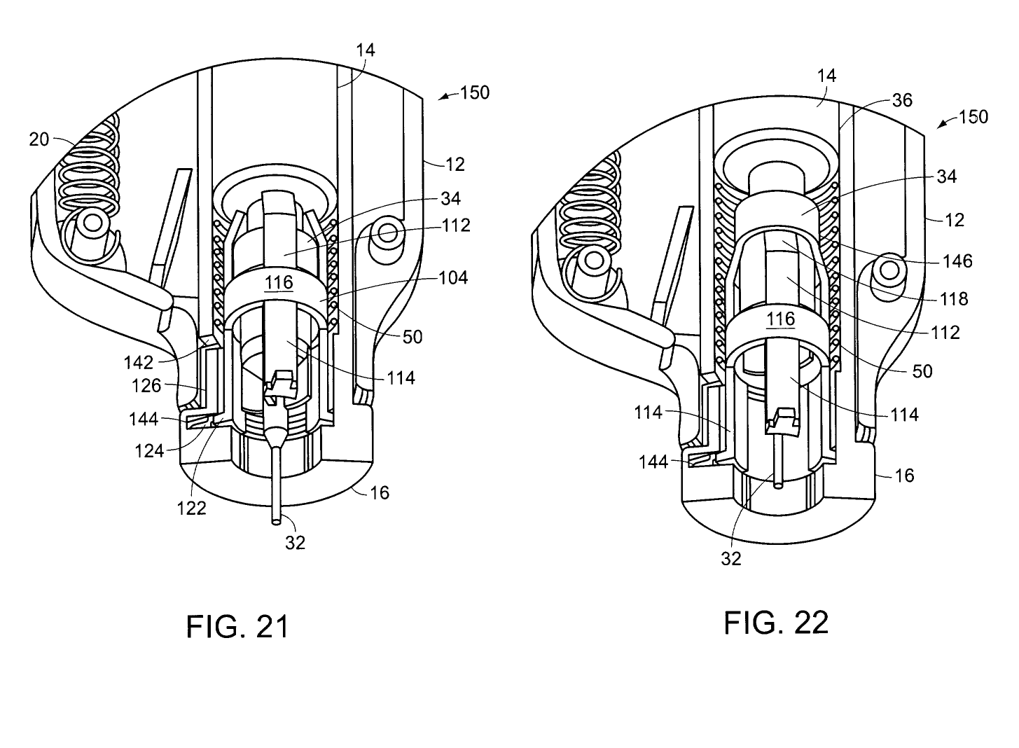

Once the trigger 24 activates the injector 10, the driving unit 20 pulls the syringe 14 and piston rod 28 forward (FIG. 17). The needle 32 is injected into to the injection site 52. The retracting spring 50 is pressed by the stronger driving unit 20 and moves forward with the syringe 14 causing the narrow section 124 to slide in the grooves 126 until reaching the front or distal end of the cartridge 16. The latches 114 are flexible and try to move out in a radial direction. Therefore, as shown in FIG. 21, when the latches 114 are moved to forward locking slots 144, the wide and narrow sections 122, 124 enter the locking slots 144 and fix the locking clip 104 in its final position.

After delivery and during retraction, the retracting spring 50 pushes the syringe 14 rearwards towards the proximate end 23 so that an outer flange 146 of the needle hub 34 slides away from the base 116 of the locking clip 104. That is, the flange 146, that was previously locked relative to the clip 104 by the fingertips 118 is pushed by the retracting spring 50 with sufficient force to urge the elastic fingers 112 radially outwards, and to slide through and past the fingertips 118. This force was previously unavailable when the retracting spring 50 was held compressed by the stronger force of the driving unit 20.

The locking clip 104 is fixed to the cartridge 1 6 and is therefore prevented from moving with the needle hub 34 when the syringe 14 is pushed by the retracting spring 50. The retracting spring 50 moves the syringe 14, causing the flange 146 to push against the inward extending fingertips 118. This pushing of the flange 146 urges the fingertips 118, and thus, the fingers 112 outward, which allows the flange 146 to slide through the outwardly pushed fingers 112. Once the flange 146 slides through, the elastic fingers 112 snap back upon the hub 34 such that the fingertips 118 abut the flange 146 and prevent axial movement of the hub 34 back towards the distal end 21. Thus the needle hub 34 slides through the fingers 112 during retraction and locks against the fingertips 118 of the fingers 112, as shown in FIGS. 18 and 22. At this position, the needle 32 is locked from moving back out of the housing 12.

Differences between injector 10 described in FIGS. 1-7 and the version described in FIGS. 15-22 are further discussed below. For example, in the exemplary embodiment shown in FIG. 1, the holding latch 26 of the piston rod 28 abuts a shoulder 30 of the housing 12 prior to activation of the injector 10. The engaging finger 54 of the control unit is already engaged with the piston rod 28 through the rear notch 40, and disengaging finger 56 rides on the extended rail 134 of the cartridge 16. The injector 10 is activated by depressing the trigger 24 of the housing 12 which biases the holding latch 26 to disconnect from the housing shoulder 30 and engages with the syringe 14. The trigger 24 is located opposite the control unit 18.

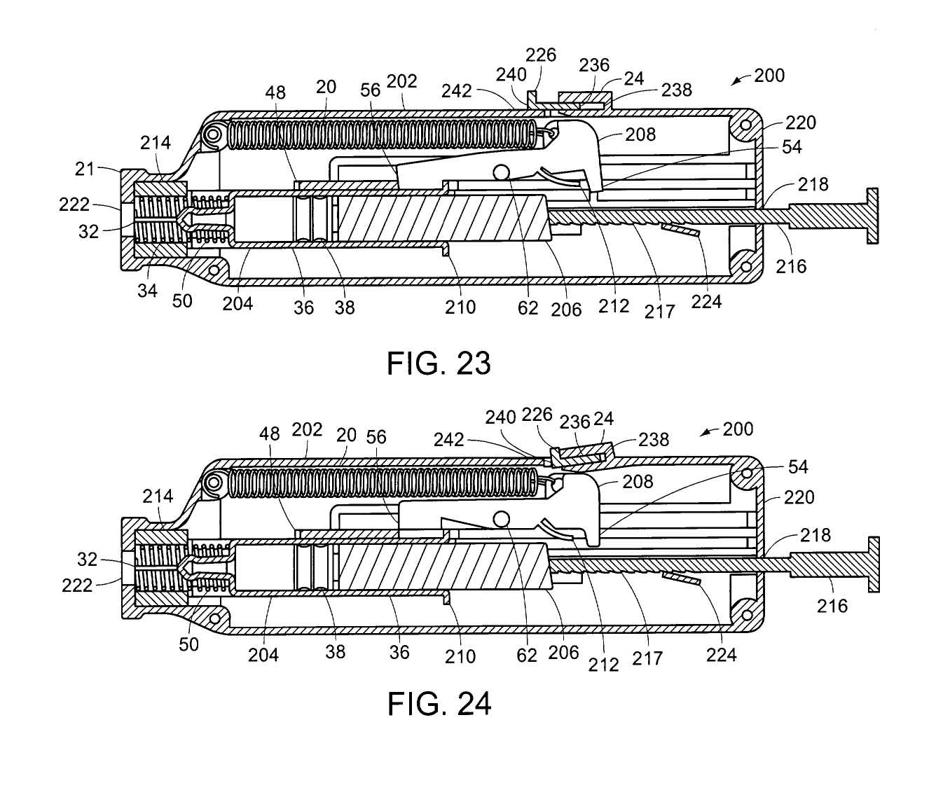

In contrast, in the exemplary embodiment shown in FIGS. 15-18, the holding latch 26 of the piston rod 28 does not hold the injector 10 under load prior to injection. Rather, the primary purpose of the holding latch 26 in this embodiment is to ensure needle penetration prior to the liquid delivery. The control unit 18 is biased by the driving unit 20, but is held by the rear wall 145 of the cartridge 16. Depressing the trigger 24 that is adjacent the control unit 18 activates the injector 10. This causes the control unit 18 to pivot around its axis 62 (not shown here, it is similar to axis 62 in another embodiment illustrated in FIG. 23). This pivot causes the control unit 18 to be engaged with the piston rod 28, and releases the control unit 18 from the rear wall 145 of the cartridge 16.

### Pre-Filled Fully Disposable Injector with Titration and Syringe Lock

Referring to FIGS. 23-29, there is shown at 200 an automatic injector constructed in accordance with another exemplary embodiment of this invention. This injector 200 is similar to the injector 10 described in FIGS. 1-7 and 15-18. In particular, the injector 200 includes a housing 202, a syringe 204, a piston rod 206, a control unit 208, a driving unit 20 and a retracting unit or spring 50. Preferably, examples of the injector 200 also includes a leaf spring 212 extending from the control unit 208, as will be described in more detail below.

The housing 202, syringe 204, piston rod 206, control unit 208, driving unit 20 and retracting spring 50 are substantially similar to the housing 12, syringe 14, piston rod 28, control unit 18, driving unit 20, and retracting spring 50 described above and thus will not be described to the extent that the elements are the same to avoid redundancy. Like-referenced numerals also designate like elements. For example, like all the injectors disclosed herein by example, the injector 200 has a distal end 21 from which the needle is exposed for delivery, and a proximate end 23 opposite the distal end 21. Without being limited to a particular theory, the term distal refers to the end or direction of the injector that is applied to the injection site for delivery, and the term proximate refers to the end or direction opposite the distal end or direction.

The injector 200 includes a bumper 214 and a guide 48. The bumper 214 is preferably a flexible and foamy shock absorber for absorbing unwanted drug and motion shocks. The guide 48 functions as a cam that controls the rotational position of the control unit 208 in a substantially similar manner that the extended rail 134, and the disengaging notch 58 of the disposable cartridge 16 control the rotational position of the control unit 18. The guide 48 controls the rotational position of the control unit 208 by preventing the control unit 208 from pivoting during needle injection and drug delivery. Without being limited to a particular theory, the guide 48 acts like a wall that allows the disengaging finger 56 of the control unit 208 to slide along the wall. At the end of delivery, the control unit 208 has slid adjacent the guide 48 and is now positioned to pivot about its axis 62 so that its disengaging finger 56 can rotate over the guide 48. The control unit 208 preferably includes a leaf spring 212, which will be described in greater detail below.

The injector 200 also includes a back rod 216 having a ratcheted surface 217 thereon. The back rod 216 extends through an opening 218 of a rear wall 220 at the proximate end of the injector 200. The back rod 216 engages with the piston rod 206 for pushing the piston rod 206, as will be described in greater detail below.

The leaf spring 212 shown in the figures extends radially inward and longitudinally toward the engaging finger 54. The leaf spring 212 terminates before reaching the engaging finger 54 leaving a space sufficient to receive the radially extending flange 210 of the syringe 204 at the back of the barrel 36. After retraction of the needle 32, the leaf spring 212 locks the syringe 204 from forward axial movement, thus locking the needle from re-exposure.

### Titration of the Device

If a safety cap 64 is provided, as shown in FIG. 1, then the first step in using this injector 200 is to remove the safety cap 64 out of the opening 222 at the distal end of the injector 200. Then any residual air in the syringe 204 should be drained out and the amount of liquid in the syringe can be adjusted to the required dosage by titration. The titration is achieved by vertically positioning the injector 200 so that the needle 32 is upright and moving the back rod 216 toward the piston rod 206 and thus, moving the unwanted drug out of the injector through the needle. In this exemplary embodiment, as shown in FIG. 23, the syringe 204 is pre-filled and prevented from moving longitudinally for injection before activation because, as shown in FIG. 23, the guide 48 prevents longitudinal movement by the disengaging finger 56 of the control unit 208 towards the distal end 21.

The bumper 214 preferably absorbs the undesired drug. The bumper 214 is preferably formed of a material, such as a foam or sponge-like rubber that absorbs both physical force and fluid.

Titration solves the problem of removing residual air commonly included in pre-filled syringes, which is a by-product of the filling technology. Titration also releases potential high static friction between the plug 38 and the barrel 36 caused by non-movement over a long period of time (i.e., storage).

Use of the Device

The housing 202 includes a rear latch 224 that engages with the ratchet edge 217 of the back rod 216 to provide for precise incremental advancement of the piston rod 206 during titration. As shown in FIGS. 23-29, the ratchet edge 217 is serrated and arranged to rub against the rear latch. However, the edge 217 and rear latch 224 are configured so that the back rod 216 can slide past the latch 224 in one direction (e.g., towards the piston rod 206), but not in the opposite direction (e.g., away from the piston rod 206). The rear latch 224 is constantly pressed against the ratchet edge 217 and stays in contact with the edge 217. Therefore, as one having ordinary skill in the art would readily understand, the latch lets the back rod 216 move towards and push the piston rod 206, but does not let the back rod 216 move back against the latch 224. Preferably a scale is visible on the syringe 204 through the housing 202 so the user can read the amount of liquid in the barrel 36. In addition, the rear latch 224 and serrated edge 217 provide a lock against rearward movement of the back rod 216.

As shown in FIG. 24, a safety tab 226 is pulled back into the trigger 24 and the trigger 24 is pushed to activate the injector 200. As can best be seen in FIGS. 23 and 24, the safety tab 226 prevents false activation of the injector 200 while the safety tab 226 is forward in its safe position.

Regarding FIG. 23, the safety tab 226 is shown having an “L” shaped cross-section. The tab 226 has a first end 236 slidingly engageable within a slot 238 of the trigger 24. Moreover, the tab 226 has a second end 240 adjacent a housing lip 242 of the housing 202. When the safety tab 226 is in its safe position (FIG. 23), the tab 226 restricts an activating movement of the trigger 24 towards the control unit 208. The trigger 24 cannot be moved towards the control unit 208 to activate the injector 200 because the second end 240 of the safety tab cannot move through the housing lip 242 while the second end 240 abuts the housing lip (FIG. 23). However, referring to FIG. 24, when the safety tab 226 is moved into the slot 238, the second end 240 of the tab 226 moves away from contact with the housing lip 242. Since the safety tab 226 is no longer in contact with the housing lip 242, the trigger 24 is free to move towards the control unit 208 and activate the injector 200.

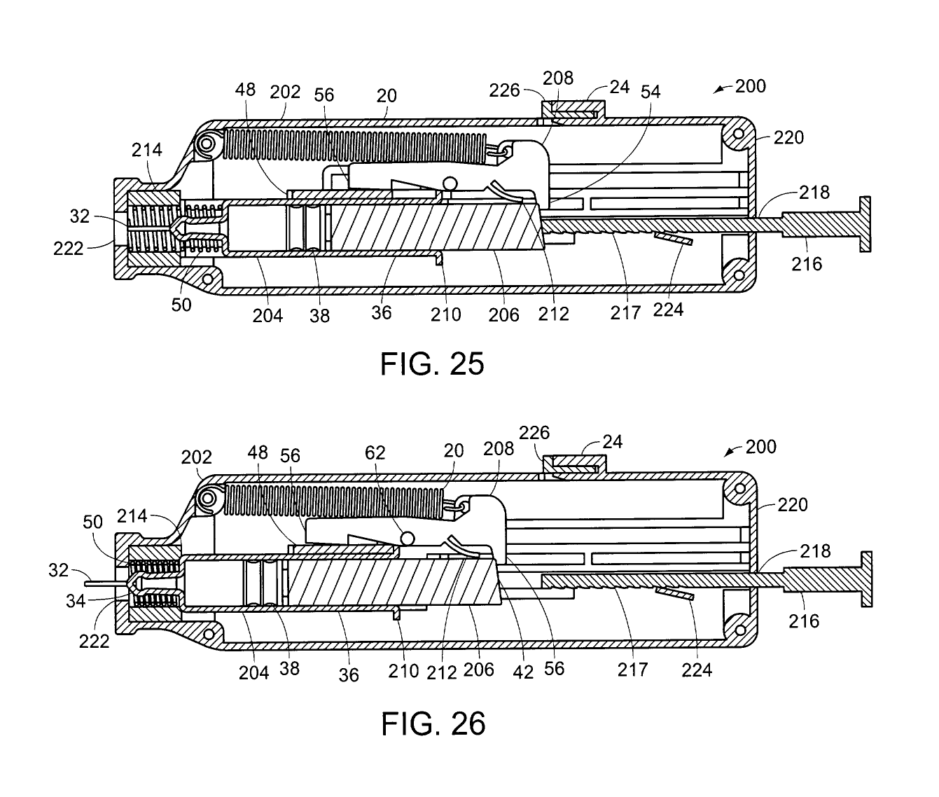

The injector 200 is pressed against the injection site 52 (FIG. 2) and the trigger 24 is pressed. This motion causes the control unit 208 to pivot on its axis 62 and release the lock of the disengaging finger 56 on the rear wall of the guide 48. Thus, the syringe 204 that was locked by the prior position of the disengaging finger is released too. Releasing of both, the disengaging finger 56 and the syringe, allows the driving unit 20 to pull the control unit 208 toward the needle 32. As the control unit 208 is pulled, it comes into contact with the piston rod 206 as shown in FIG. 25. The driving unit 20 provides a force stronger than an offsetting force of the retracting spring 50, which permits the driving unit 20 to push the liquid through the needle 32. As shown in FIG. 26, the biasing of the control unit 208 on the piston rod 206 moves the syringe 204 forward, causing needle penetration. The syringe 204 moves forward until it contacts the bumper 214 that is arranged to deflect the force of the blow of the syringe 204 to the patient. Once the syringe 204 moves to its forward position, the driving unit 20 continues to pull the control unit 208 and force the fluid in the syringe 204 through the needle 32 for delivery.

Without being limited to a particular theory, as an example of the balances of forces working in the injector, it generally takes about 0.9 kgf to deliver 1 mm. of fluid over 5 seconds via a number 28 sized needle. The preliminary pulling force of the driving unit 20 is, for example, 1.6 to 1.8 kgf, and the final pulling force of the driving unit 20 is 0.7 to 0.9 kgf. Dynamic friction will take, for example, 0.2 kgf in maximum. The retracting spring 50 will require, for example, about 0.06 to 0.08 kgf to allow the syringe 204 to fully compress the spring.

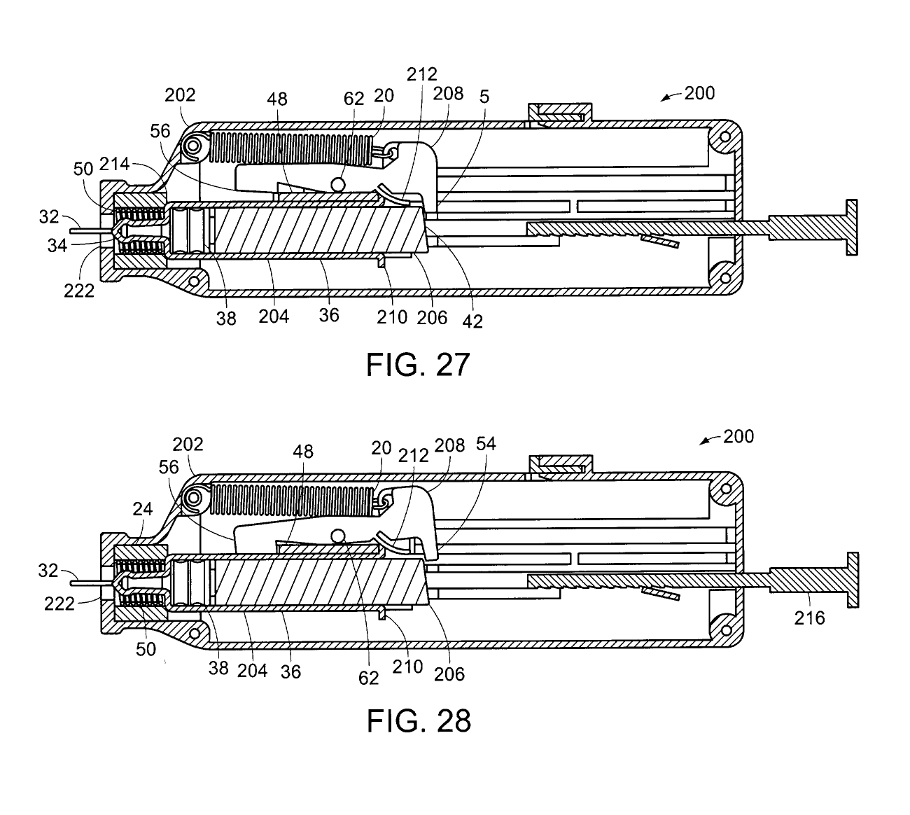

FIG. 27 shows the injector 200 upon end of delivery. As shown, in order to make sure that effects of tolerances do not affect the ability to dispense the entire drug solution out of the syringe 204, the flexible bumper 214 is used as a tolerance absorber for compensation. The static force on the bumper 214 is greater than the force from the syringe 204 at the end of delivery. That is, since the piston rod 206 is bottomed at the end of delivery, the balance of forces in the injector 200 becomes static and therefore the reaction on the bumper 214 is greater. Accordingly, the flexible bumper is used as a tolerance absorber.

At this moment, the disengaging finger 56 of the control unit 208 is no longer restricted in its movement by the guide 48. The driving unit 20 continues to pull on the control unit 208, causing the control unit 208 to pivot about its axis 62. This pivot causes the disengaging finger 56 to slide around the guide 48 towards the barrel 36 and releases the engaging finger 54 from behind the piston rod 206, thereby releasing the syringe 204 from the forces of the driving unit 20. As shown in FIG. 28, when the control unit 208 completes its pivot, the syringe 204 is free to retract because there are no substantial forces preventing the retracting spring 50 from pushing the syringe 204 back into the housing 202. It is important to note that the syringe 204 includes the flange 210 extending radially outward from the back end of the barrel 36.

The leaf spring 212 does not affect movement of the control unit 208 or the syringe 204 before or during delivery. However, during retraction the syringe flange 210 by-passes the leaf spring 212 and is locked between the leaf spring 212 and the engaging finger 54 of the control unit 208, preventing potential axial movement of the syringe and consequential re-exposure of the needle 32. In other words, the syringe 204 is locked and unable to move forward beyond the leaf spring 212 to expose the needle 32.

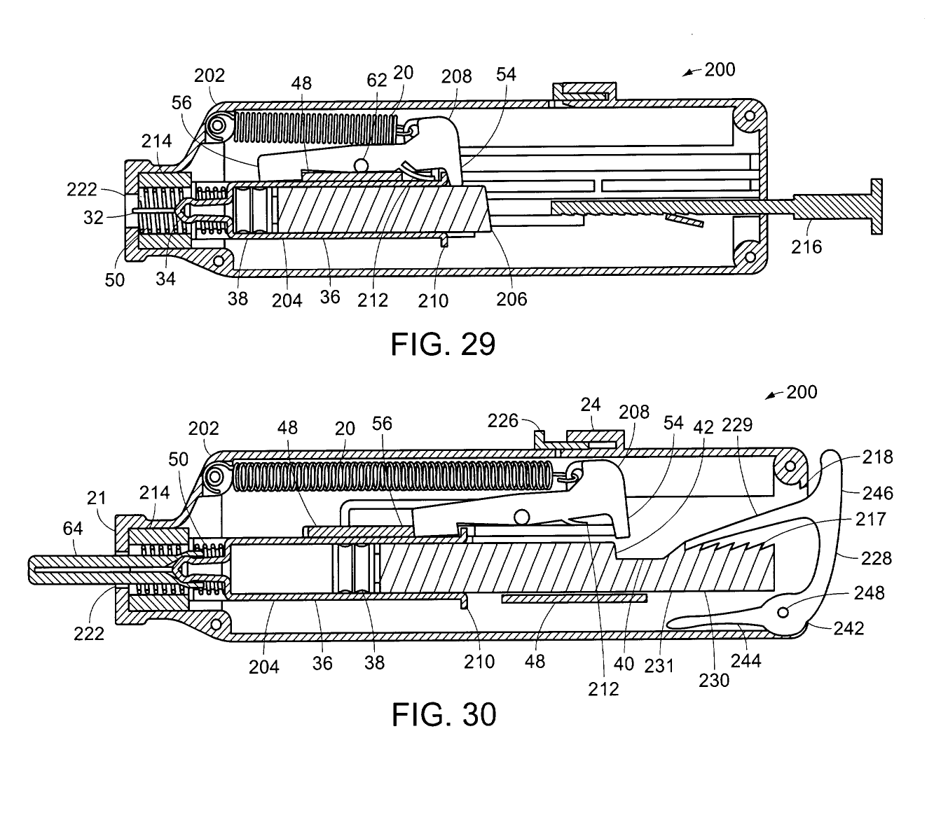

FIG. 29 shows the injector after retraction. As shown, the needle 32 is within the housing 202, and cannot be forced back through the opening 222 because the syringe 204 is locked by the leaf spring 212. In particular, the syringe flange 210 is locked between the leaf spring 212 and the engaging finger 54, thereby locking the syringe 204 and needle 32 from re-exposure. The leaf spring 212 extends radially inward and longitudinally rearward toward the engaging finger 54. The leaf spring 212 terminates before reaching the engaging finger 54 leaving a space sufficient to receive the radially extending flange 210 at the back of the barrel 36. After retraction, the leaf spring 212 locks the syringe 204 from forward axial movement, thus locking the needle 32 from re-exposure.

During retraction, the syringe flange 210 by-passes the leaf spring 212 and is locked between the leaf spring 212 and the engaging finger 54 of the control unit 208, preventing potential re-exposure of the needle 32. In other words, the syringe 204 is locked and unable to move forward beyond the leaf spring 212 to expose the needle 32.

FIG. 30 illustrates another example of this exemplary embodiment. In this example, a pawl 228 is used in place of the back rod 216 described above. The pawl 228 includes a pawl hub 242, a spring support 244, a pawl lever 246 and a pawl arm 229. Preferably, the pawl hub 242 is pivotally attached to the housing 202 via a housing pin 248. That is, the pawl hub 242 is arranged to allow the pawl 228 to pivot about the housing pin 248 and move the piston rod 230 forward for titration. The spring support 244 extends from the pawl hub 242 and contacts the housing 202. The spring support 224 is arranged to contact the housing 202 as necessary to keep the pawl 228 in position to act on the piston rod 230. The pawl lever 246 extends from the pawl hub 242 outside of the housing 202. The lever 246 is arranged to act on the piston rod 230 upon receipt of an outside force (e.g., human pressure) on the pawl lever 246. The pawl arm 229 extends from the lever 246 for communication with the piston rod 230, as will be described in greater detail below.

The piston rod 230 is a modification of the piston rod 206 described in FIGS. 23-29. In particular, the piston rod 230 includes a back section 231 having a rear notch 40 and a serrated edge 217. The serrated edge 217 communicates with the pawl arm 229 to provide a ratchet mechanism arranged to permit motion of the piston rod 206 in one direction towards the injection needle 32. The pawl 228 can move the piston rod 230 forward towards the distal end 21 and can lock the piston rod 230 from rearward movement, thereby providing an alternative structure for the back rod 216 and piston rod 206 described above.

### Empty Fully Disposable Injector with Mixing, Titration and Reconstitution

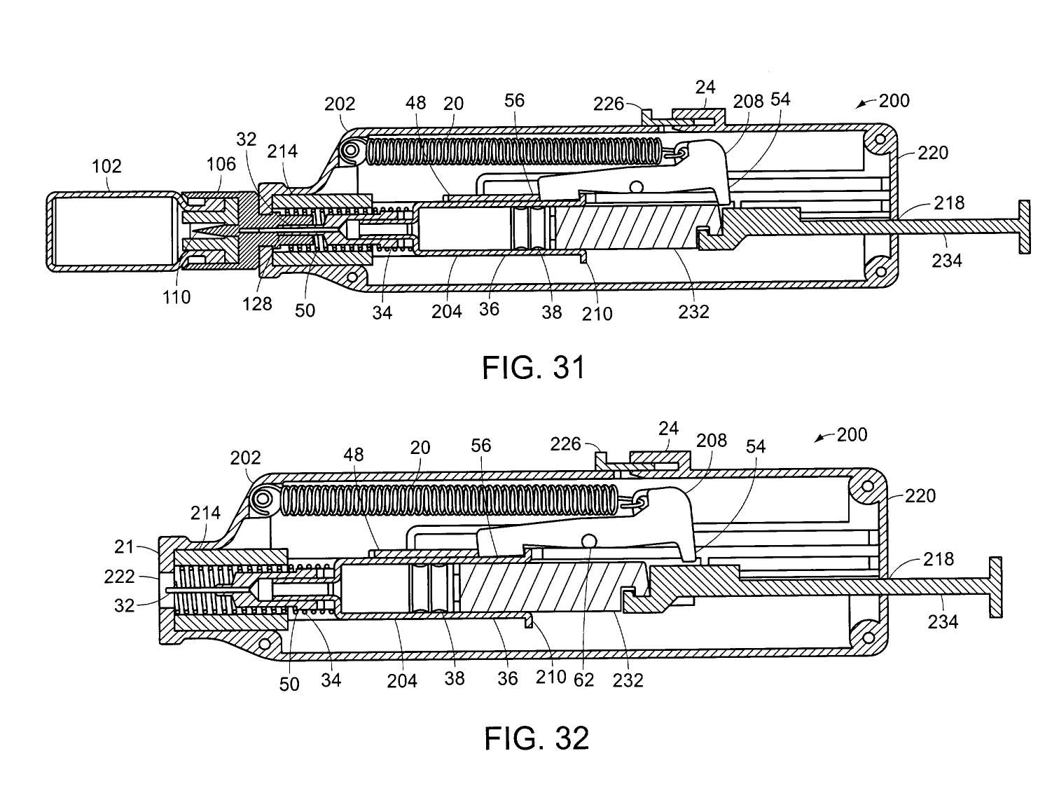

FIGS. 31-36 show an injector constructed in accordance with another exemplary embodiment of the present invention. This embodiment is substantially similar to the previously discussed embodiments but for the piston rod 206 and back rod 216. In this exemplary embodiment, a piston rod 232 and back rod 234 can be interlocked, and the back rod 234 is disengaged from the piston rod 232 by the engaging finger 54 upon rotation of the control unit 208. Since the piston rod 232 and back rod 234 are interlocked, the back rod 234 can push and pull the piston rod 232 to perform aspiration, titration and reconstitution before injection. The leaf spring 212 is not shown in the sectional views of FIGS. 31-36 for even better visual clarity of the relationship between the control unit 208, the piston rod 232 and the back rod 234. The leaf spring 212 does not affect any of the movement within the injector 200 as shown in FIGS. 31-36.

FIG. 31 is a sectional view of the injector 200 after the syringe 204 is filled with the liquid from the vial 102. The syringe 204 is filled by pulling the back rod 234 away from the vial 102. The back rod 234 is slidingly interlocked with the piston rod 232. Pulling the back rod 234 pulls the piston rod 232, creating a vacuum m the barrel 36 for sucking the drug liquid from the vial 102. The vial 102 and adapter 106 are removed from the injector 200 and drug titration is provided by pushing the back rod 234, into the housing 202 which urges the piston rod 232 into the barrel 36, thereby pushing excess liquid out of the syringe.

Without being limited to a particular theory, it is within the scope of this invention that the injector 200 can also provide reconstitution. For example, referring to FIG. 31, after the liquid is transferred from the vial 102 to the syringe 204, the vial 102 is removed from the adapter 106 and replaced with a second vial 102. The second vial 102 contains a lyophilized or solid drug, and is attached to the adapter 106. The liquid in the syringe 204 is then transferred into the second vial 102, preferably by pushing the back rod 234 into the housing 202. The back rod 234 urges the piston rod 232 into the barrel 36, which moves the fluid through the needle 32 into the second vial 102. The fluid mixes with the lyophilized drug in the vial 102 to form a reconstituted liquid drug. The reconstituted liquid drug can be drawn into the syringe 204 by pulling the back rod 234 away from the vial 102, as described above. It is also within the scope of this invention that liquids could be mixed using the embodiment shown in FIG. 31, by moving the liquids between the syringe 204 and vials 102 attached thereto, as a skilled artisan would readily understand.

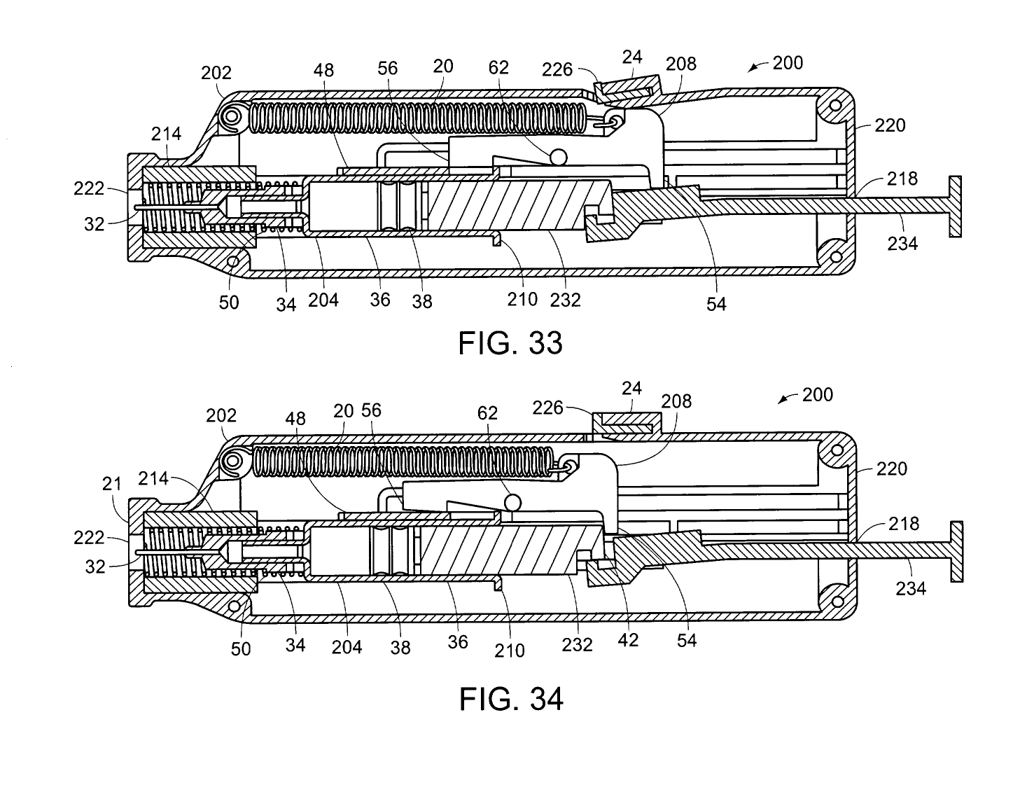

When the drug solution is ready for injection, the injector 200 is ready for activation. Preferably, the injector 200 is attached to the injection site 52. Then, as shown in FIG. 33, the safety tab 226 is moved under the trigger 24 and the trigger 24 is pressed to activate the injector 200. This operation can easily be provided with one hand.

Once the trigger 24 is pushed, it urges the control unit 208 to pivot on its axis 62. The pivot moves the disengaging finger 56 away from the guide 48 and the syringe 204 and releases its hold on the syringe 204. Further, referring to FIG. 33, the pivot of the control unit 208 causes the engaging finger 54 to push the back rod 234 out of its interlocking engagement with the piston rod 232. The urging of the control unit 208 against the back rod 234 automatically causes separation, releasing the back rod 234 from the piston rod 232. That is, the separation is not provided manually, but is automatically provided upon activation by the urging of the control unit 208 against the back rod 234.

As shown in FIG. 34, the driving unit 20 pulls the control unit 208, closing the gap between the engaging finger 54 of the control unit 208 and the piston rod 232. The acceleration of the control unit 208 caused by the driving unit 20 may cause an impact from the engaging finger 54 hitting the piston rod 232 that is large enough to cause the piston rod 232 to push a small amount of drug out of the syringe 204 prior to injection which would result in a slight loss of volume of the desired dosage. This problem is easily solved by adding a dampening effect for that movement, based on the strength of the driving unit 20 during this stage.

As an example of a dampening effect, the plug 38 preferably includes rubber and is resilient to react to the impact of the driving unit 20 on the syringe 204 by expanding and increasing the friction to the barrel 36. That way the syringe 204 continues to move toward the distal end 21 of the injector 200 until penetration is achieved. From that point the driving force of the driving unit 20 overrides that friction and pushes the drug through the needle 32. In other words the plug 38 is soft enough to react to the impact by expanding and increasing the friction to the barrel 36. That way the syringe 204 continues to move forward as a whole till penetration is achieved. From that point the driving force provided by the driving unit 20 overcomes that friction. The same principle of adding friction with the plug 38 can also be adapted in the other embodiments, including the embodiments using a pre-filled syringe 204.

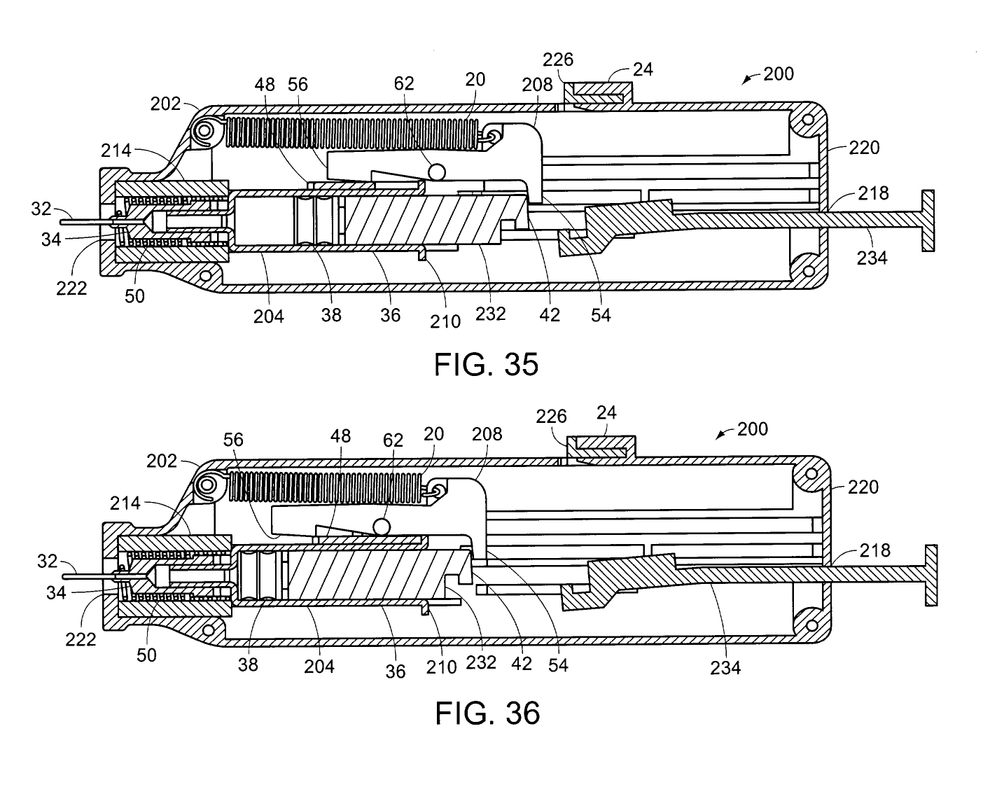

As shown in FIGS. 35 and 36, the injector 200 then operates in the same manner as explained in the exemplary embodiments described above. That is, the driving unit 20 pulls the control unit 208, which urges the piston rod 232 through the barrel 36, and moves fluid out of the syringe 204 into the injection site for delivery. Further, the syringe 204 automatically retracts at the end of delivery, as shown for example in FIGS. 28 and 29.

### Empty Fully Disposable Injector with Mixing, Titration, Reconstitution, Syringe Lock and Body Sensor

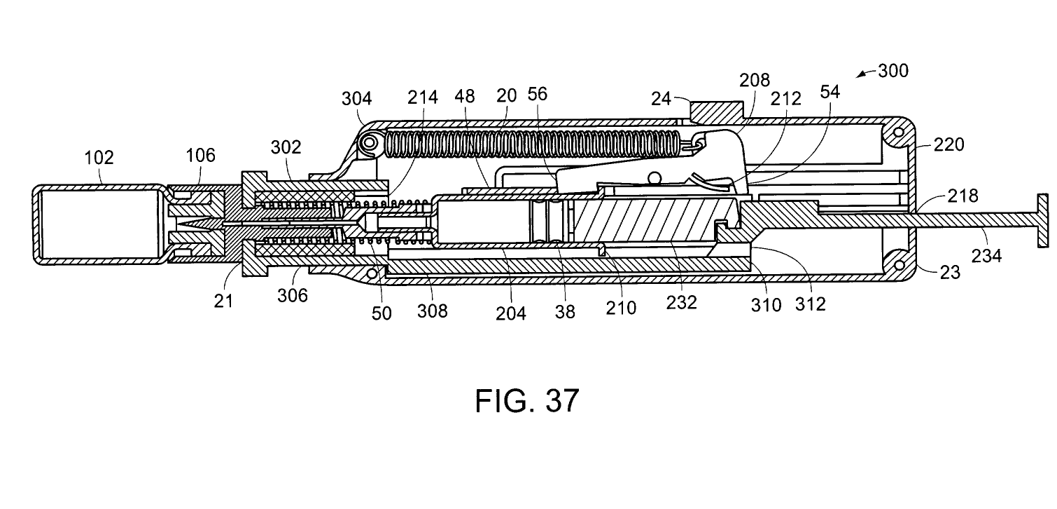

An injector 300 in accordance with yet another exemplary embodiment is shown by example in FIGS. 37 through 47. This injector 300 differs slightly from the previous injectors discussed above in that this injector 300 includes a body-sensing unit 302 having a sleeve 306 that prevents premature activation of the injector 300. The exemplary body-sensing unit 302 shown in the figures prevents premature activation by preventing an ill-timed activating movement by the trigger 24, thereby providing the benefit of the safety tab 226 discussed above. However, while the safety tab 226 is moved by the user, the body sensing unit 302 provides a safety feature by enabling injection activation by pressing the injector 300 against the injection site, as will be described in more detail below. In summary, pressing the distal end 21 of the injector 300 against the injection site moves the unit 302 out of contact with the control unit 208, thereby freeing the control unit 208 to pivot when the trigger 24 is depressed. Of course, the body sensing unit 302 could be included in the other exemplary embodiments described herein, as readily understood by a skilled artisan.

Referring to FIGS. 37 through 47, and in particular to FIG. 37, the injector 300 includes a housing 304, a syringe 204, a control unit 208, a driving unit 20, a retracting spring 50, a piston rod 232 and a back rod 234. The housing 304 is similar to the housing 202, but is configured to accept the unit 302 through an opening 305. The body-sensing unit 302 of the injector has a cartridge sleeve 306 slidingly engaged within the distal opening 222 of the housing 304. The bumper 214, retracting spring 50, needle 32 and needle hub 34 are shown within the sleeve 306. Moreover, the vial adaptor 106 and vial 102 are attached to the sleeve 306 and the needle 32 for filling the syringe 204, preferably with the syringe 204 in an upright vertical position, with the distal end 21 above the proximate end 23, to ensure air stays above any liquid. Regarding FIG. 37, the injector 300 includes the vial 102, adapter 106, syringe 204, piston rod 232 and back rod 234 substantially as shown above in FIG. 31. Therefore this injector 300 can also be used for titration, mixing and reconstitution, as readily understood by a skilled artisan. Of course, it is understood that this injector 300 could also be used with a prefilled syringe 204.

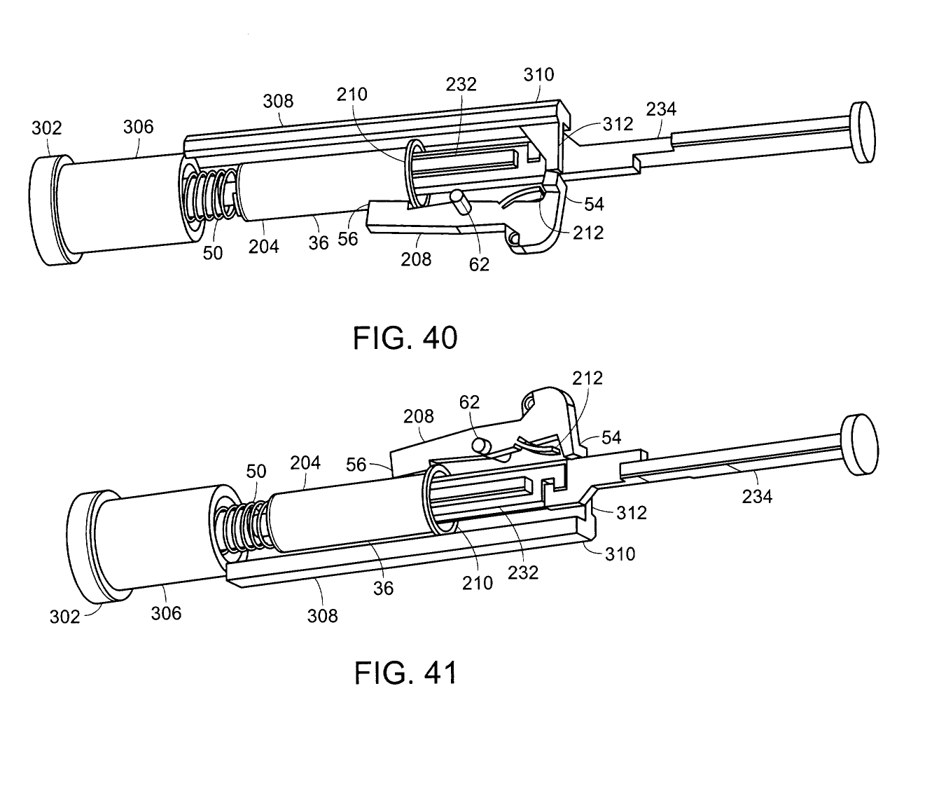

The body-sensing unit 302 also includes a leg 308 extending longitudinally rearward from the cartridge sleeve 306 along an interior wall of the housing 304. The leg 308 has a proximate end 310 from which a foot 312 extends radially inward. Before the injector 300 is activated, the foot 312 abuts the engaging finger 54 of the control unit 208. By abutting the engaging finger 54, as can be seen in FIGS. 40 and 41, the foot 312 prevents movement of the control unit 208 and locks the trigger 24 from activating the injector 300.

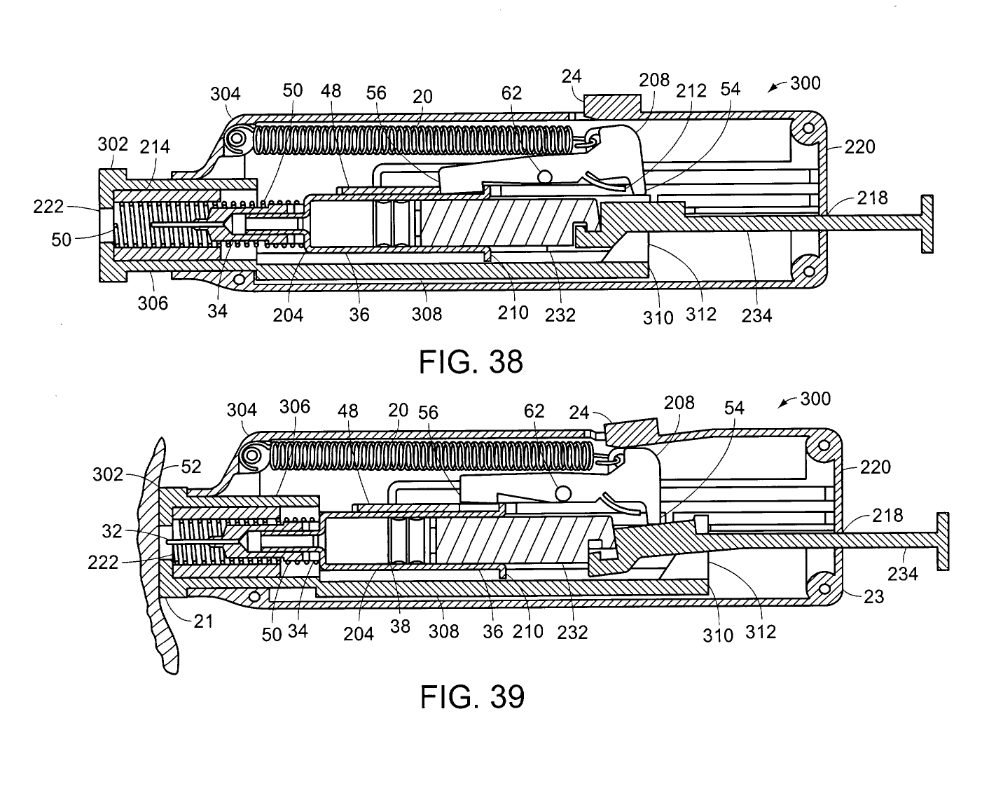

FIG. 38 shows the injector 300 ready for use. As with the other exemplary embodiments, it is understood that either the drug is aspirated into an empty syringe 204 or the injector 300 is manufactured with a pre-filled syringe 204, as described in more detail earlier with respect to FIGS. 1 to 36. The syringe 204 is locked from forward axial movement by the disengaging finger 56 of the control unit 208. However, it is understood that the locking clip 104 disclosed above could be implemented in this embodiment to lock the needle 32, and thus the syringe 204, before and after use. The back rod 234 and piston rod 232 are free to move to adjust the dose as desired. Accidental pressure applied to the trigger 24 will not falsely activate the injector 300 because the foot 312 of the body-sensing unit 302 prevents rotation of the control unit 208, which prevents movement of the syringe 204.

### The Injector in Use

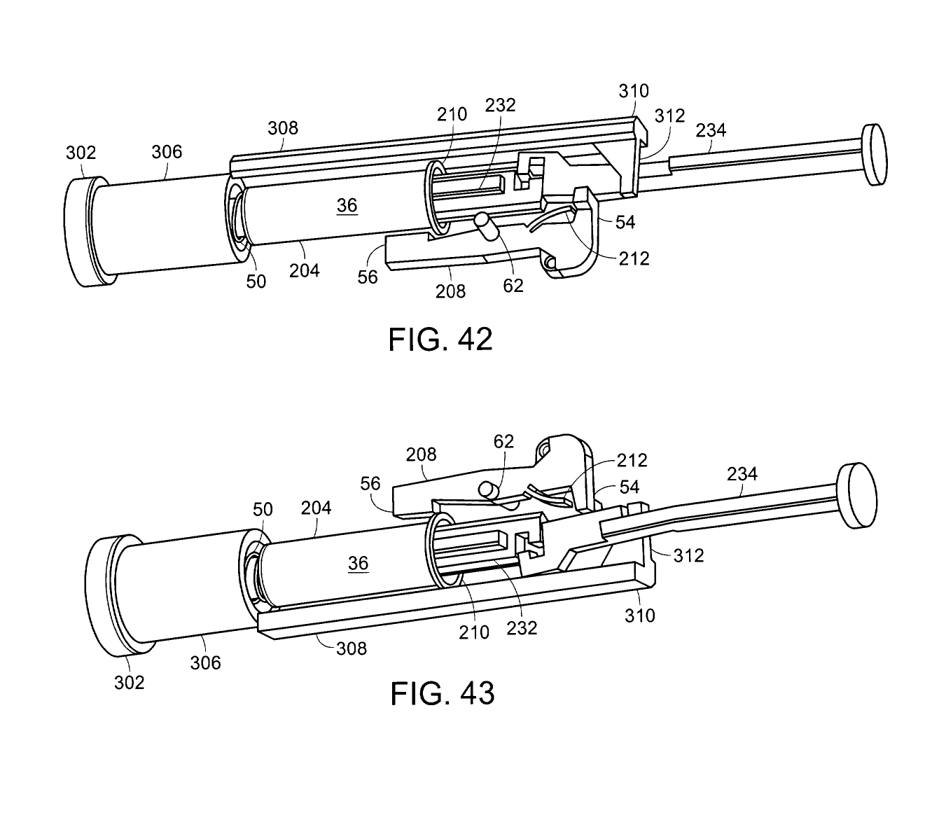

Referring to FIG. 39, when the injector 300 is pressed against the injection site 52 the applied pressure moves the body-sensing unit 302 into the housing 304, the foot 312 moves toward the proximate end 23 of the injector 300 and disengages from the control unit 208 (FIGS. 42-43). In other words, before the unit 302 is moved into the housing (FIG. 38), the foot 312 abuts the engaging finger 54 of the control unit 208, preventing pivotal movement of the control unit 308 towards the foot 312. However, after the cartridge is moved (FIG. 39), the foot 312 is no longer in contact with the engaging finger 54 and the control unit 208 is free to pivot. Upon this disengagement, pressing the trigger 24 pivots the control unit 208. The control unit automatically separates the back rod 234 from the piston rod 232, and engages the piston rod 232 to move the fluid through the needle 32 for delivery. The relationship between the foot 312, the control unit 208, the back rod 234 and the piston rod 232 is best seen in FIGS. 40-43.

FIGS. 40 and 41 are exemplary isometric views of the inner mechanism inside the injector 300 at the pre-activation stage. The figures show the body-sensing unit 302, the syringe 204, the control unit 208, the piston rod 232, the back rod 234 and the retracting spring 50. As illustrated, the foot 312 of the body-sensing unit 302 prevents rotation of the control unit 208 and engagement with the piston rod 232.

FIGS. 42 and 43 are isometric partial views similar to the views shown in FIGS. 40 and 41. However the isometric views of FIGS. 42 and 43 show the injector 300 in a state when the injector 300 is pressed against an injection site and the trigger 24 (not shown) is depressed. As noted above, the body-sensing unit 302 moves rearward when the injector 300 is pressed against the injection site 52. Then the control unit 208 can be rotated by the trigger 24 to separate the back rod 234 and begin needle injection and drug delivery.

While not being limited to a particular theory, the exemplary body-sensing unit 302 does not interfere with the operation of the injector 300. In fact, if, for any reason, the injector 300 is removed from the injection site 52 before the end of delivery, the retracting spring 50 would push the body-sensing unit 302 over the needle 32. Accordingly the needle 32 would not be exposed.

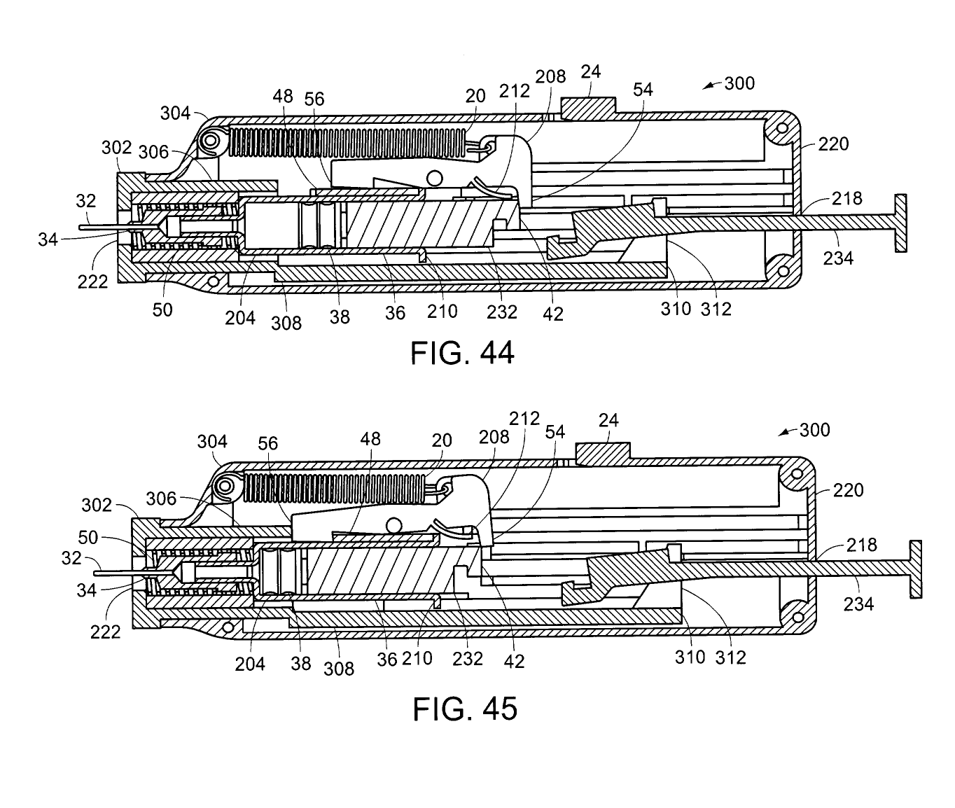

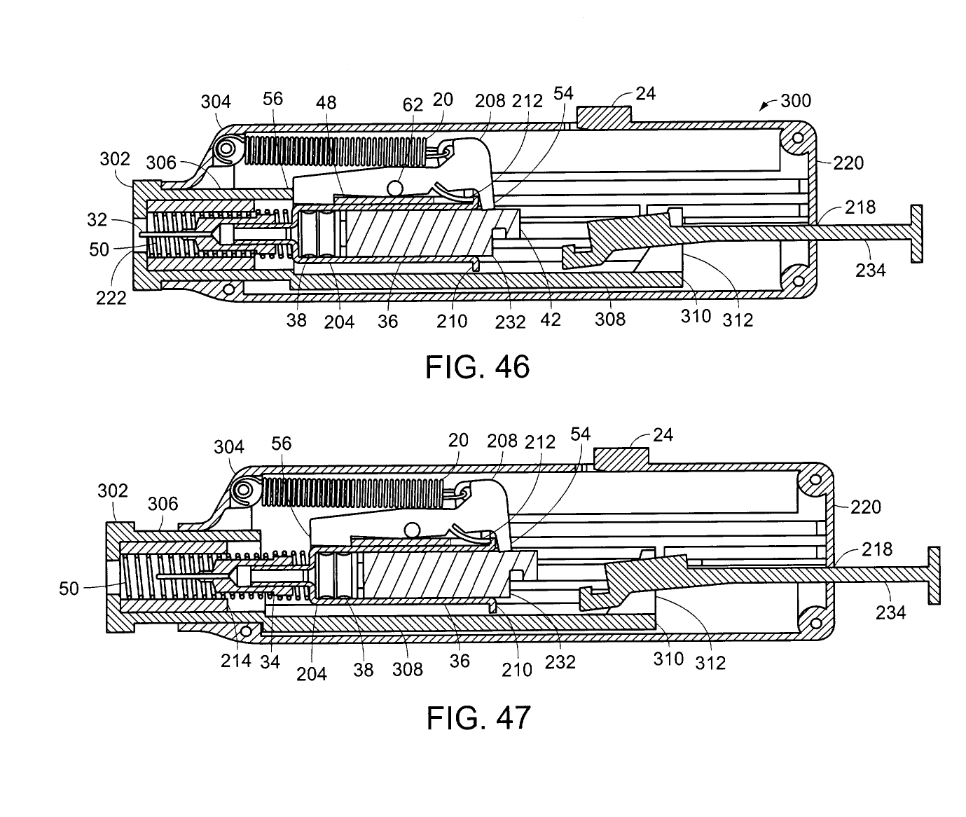

FIGS. 44 through 47 are sectional views of the injector 300 at various stages during use of the injector 300. In FIG. 44, the needle 32 penetrates the injection site 52 and delivery begins. By the end of delivery, the control unit 208 is able to pivot and disengage from the piston rod 232. At FIG. 45, the pivoting of the control unit 208 occurs, which separates the control unit 208 from the piston rod 232 and thus allows the retracting spring 50 to push the needle 32 back into the housing 304 (FIG. 46). At FIG. 47, the injector 300 is pulled away from the injection site and the retracting spring 50 urges the body-sensing unit 302 and away from the housing 304. This movement of the body-sensing unit 302provides added security from the needle 32 by adding distance between the needle 32 and the needle opening 222.

Although the length of this injector 300 may be slightly longer than previously described injectors, this injector 300 provides the advantages of: removing the user step of releasing the safety tab 226; ensuring that the injector 300 delivers the drug to the injection site 52 only after the injector 300 is attached to the site 52; and preventing exposure of the needle 32 even if the injector 300 is removed from the injection site 52 before the end of delivery.

### Disposable Pre-Filled, Dual Chamber Cartridge for Lyophilized Drug

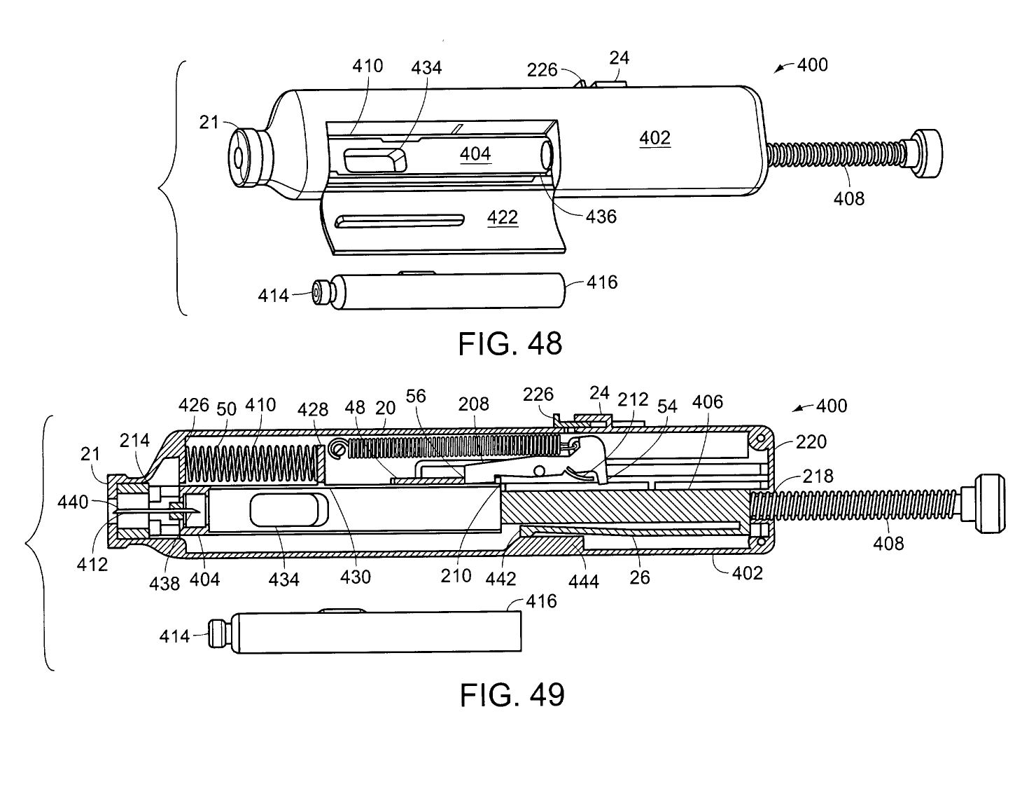

Referring to FIGS. 48 through 56, there is shown at 400 an injector constructed in accordance with yet still another exemplary embodiment of the invention. The injector 400 includes a housing 402, a tray 404, a cartridge (dual chamber or single chamber) 416, a piston rod 406, a control unit 208, a driving unit 20, a back rod 408 and a retracting unit 410 similar to the like elements described above. However, a double-sided needle 412 is preferred, with a distal end 440 for penetrating the injection site 52 and a proximate end 438 for penetrating the cartridge septum 414 as will be described in greater detail below. Also, in this exemplary embodiment, the retracting unit 410 (e.g., retracting spring 50) is shown not coaxial with the needle 412.

The tray 404 is configured to slide longitudinally within the housing 402, as will be described below in greater detail. The tray 404 is also configured to hold the cartridge 416 within the housing 402. As shown in FIG. 48, the tray 404 has a semi-cylindrical channel 436 having a radius slightly greater than the radius of the cartridge 416. In other words, the channel 436 has a size and shape that allows the cartridge 416 to seat and slide in the channel 436. A window 434 extends through the housing 402 and tray 404. A user can look through the window 434 to see if a cartridge 416 is located in the injector 400.

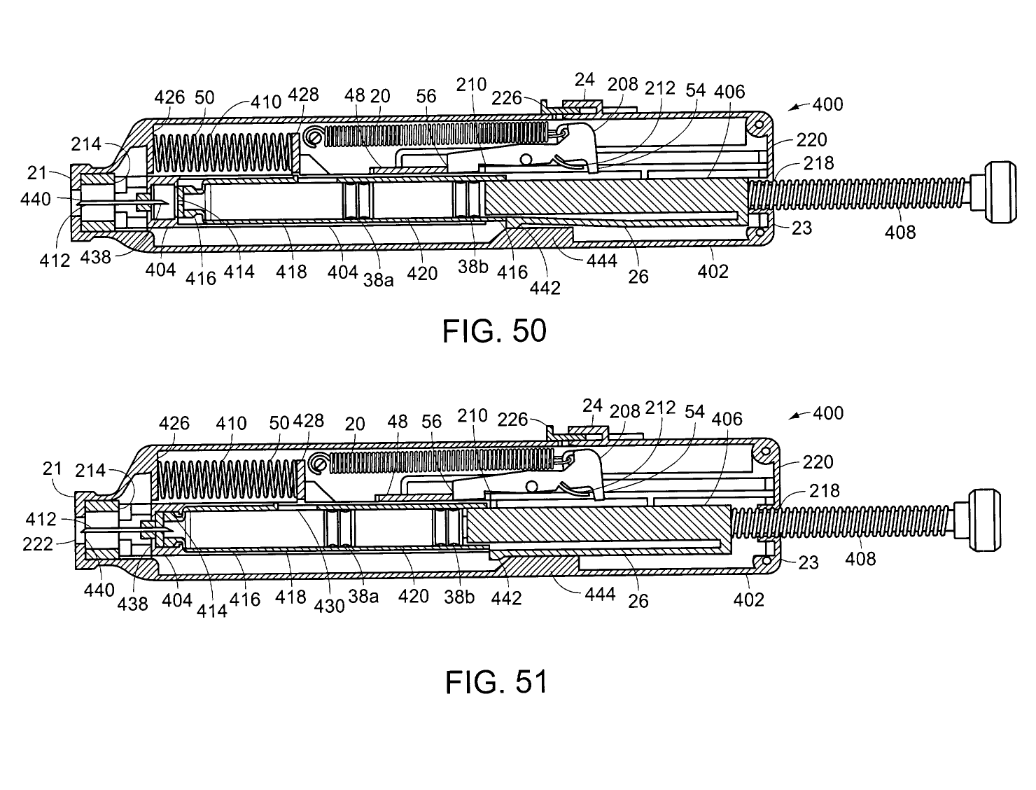

The drug cartridge used in this example is a pre-filled lyophilized dual-chamber cartridge 416. As shown in FIG. 50, a dual-chamber cartridge 416 has two compartments, a front chamber 418 typically containing a lyophilized drug, and a rear chamber 420 typically containing a diluent. To reconstitute the drug, the cartridge 416 is inserted into a side door 422 of the injector 400, as shown in FIG. 48. It is understood that the injector 400 is also suitable for use with dual chamber cartridges that include two liquids, instead of a lyophilized drug and a diluent. The combination of lyophilized drug and diluent are provided for illustration purposes, as clearly other combinations are available and useable within the scope of this invention.

FIG. 49 is a partial sectional view corresponding with the injector 400 shown in FIG. 48, e.g., before the cartridge 416 is inserted into the housing 402. FIG. 50 is section view showing the cartridge 416 located in the tray 404. As noted above, the injector 400 includes a housing 402, a tray 404, a piston rod 406, a back rod 408, a control unit 208, a driving unit 20, a retracting unit 410 (e.g., the retracting spring 50), a safety tab 226, a bumper 214 and the cartridge 416. The exemplary cartridge 416 has two chambers, one containing a lyophilized drug, and the other containing a diluent.

While similar to the injectors discussed above, the injector may be configured to include the tray 404, the two-sided needle 412, as shown in FIG. 49 and the cartridge 416. The tray 404 is arranged adjacent the needle 412 to support the needle 412. The tray 404 is arranged to allow the cartridge 416 to move axially toward the needle 412 for needle penetration into the cartridge 416, as will be described in greater detail below.

Still referring to FIG. 50, the retracting unit 410 is located eccentric to the tray 404 and the needle 412 to allow ingress and egress of the cartridge 416. As shown, the unit 410 is preferably a compression retracting spring 50 located between a static interior wall 426 of the housing 402 and a sliding wall 428 that moves longitudinally with the tray 404. The sliding wall 428 is fixed to the tray 404, and extends radially from the tray 404 toward the housing 402. The sliding wall 428 is arranged to move in the housing 402 with the tray 404. In other words, as the tray 404 moves forward towards the distal end 21, the sliding wall 428 also moves accordingly, and compresses the retracting spring 50.

FIGS. 50-57 illustrate the steps for drug delivery with the exemplary injector 400 of this exemplary embodiment. As shown in FIGS. 50, the user inserts the cartridge 416 into the housing 402 and closes the side door 422. The side door 422 can be fastened to the housing 402, for example, by snap-fitting the door with the housing. Other well-known devices can be used for fastening the door 422 with the housing, such as a latch, bolt, bar, catch, clamp or clasp, as readily understood by a skilled artisan. The injector 400 is then preferably held vertically upward so that the needle 412 is above the back rod 408. The back rod 408 is rotated in a desired direction to engage threads on the rod 408 with matching threads within the housing and push the piston rod 406 forward towards the needle 412. While the back rod 408 is threaded and rotated to urge the piston rod 406 toward the cartridge 416 in this embodiment, it is understood that the back rod 408 can also be linear or ratcheted as described above to urge the piston rod 406 toward the cartridge 416.

As shown in FIG. 50, the piston rod 406 includes a holding latch 26 having a thickened end 442. The holding latch 26 is arranged to move the cartridge 416 towards the needle 412 as the piston rod 406 is moved towards the needle. At this stage, if the piston rod 406 is urged by the back rod 408, the holding latch 26 urges the cartridge 416 forward towards the needle 412. The holding latch 26 abuts the cartridge 416 in FIG. 50 as its thickened end 442 is adjacent a projected portion 444 of the housing 402. Then the thickened end 442 is not adjacent the projected portion 444, and the holding latch 26 is allowed to flex radially outward and to separate from the cartridge 416 (FIG. 51).

Even though the cartridge 416 is placed within the tray 404, the tray 404 does not move axially with the cartridge 416 at this time because the tray 404 is held in its axial position by the disengaging end 56 of the control unit 208. The tray 404 preferably includes a radially extending flange 210 that is held in place by the disengaging finger 56 of the control unit 208, as shown in FIGS. 50 and 51.

Referring to FIG. 51, the cartridge 416 is pushed forward by the holding latch 26 (FIG. 50) relative to the tray 404. The proximate end 438 of the needle 412 penetrates the septum 414 of the cartridge 416. As the back rod 408 pushes the piston rod 406 forward so that the thickened end 442 is not adjacent the projected portion 444 of the housing 402, the holding latch 26 moves radially outward separating from the cartridge 416. As shown in FIG. 51, the piston rod 406 is now free of the cartridge 416, thereby allowing the piston rod 406 to push the contents in the cartridge 416 forward.

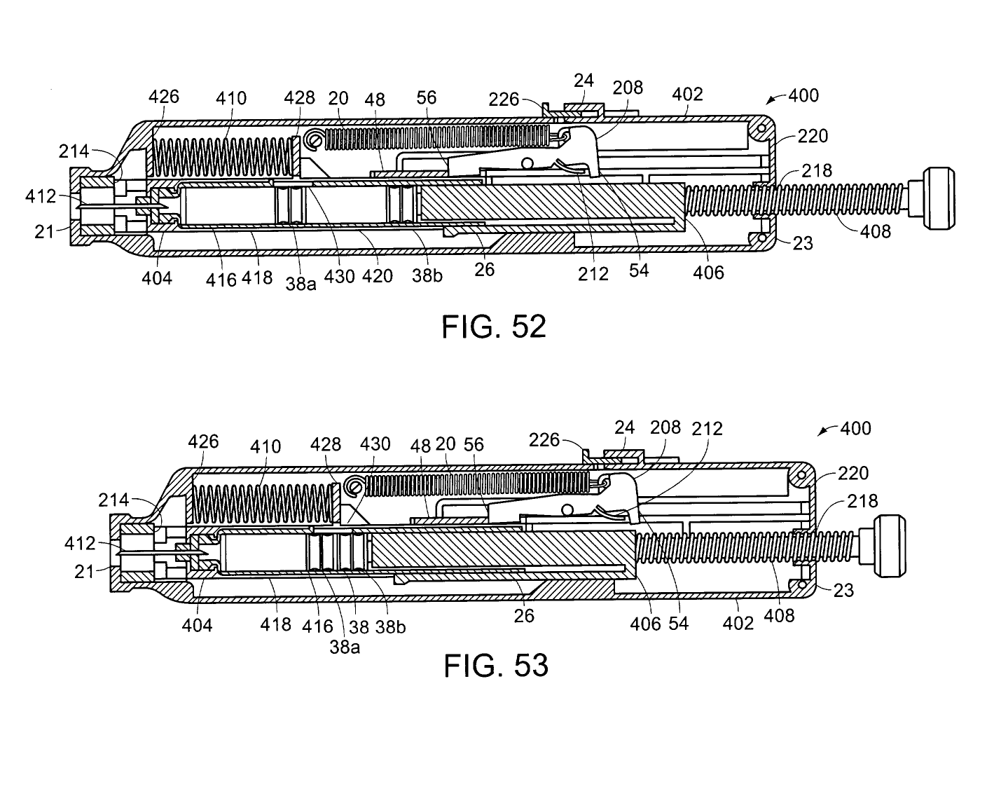

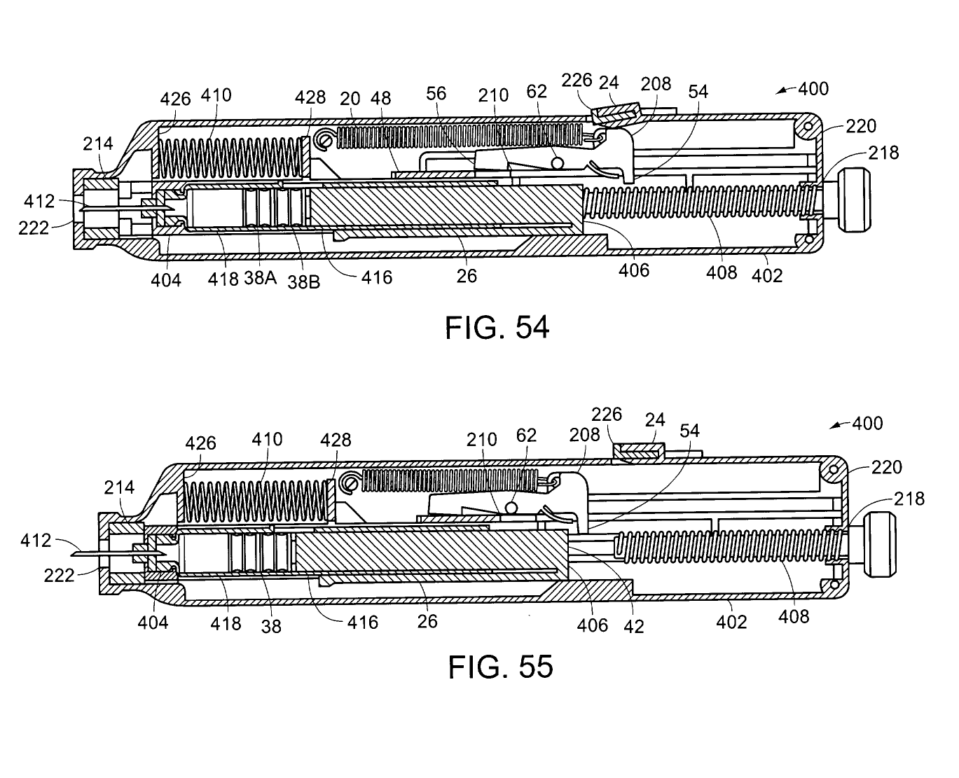

While not being limited to a particular theory, the injector 400 is preferably held in a vertically upright position with the distal end 21 above the proximate end 23 during reconstitution and titration, discussed below. As the back rod 408 is further rotated to urge the piston rod 406 forward, a channel 430 in the cartridge 416 comes into communication with the interior of both of the chambers 418,420. As shown in FIGS. 52 and 53, the piston rod 406 pushes the diluent forward into the front chamber 418 and the diluent mixes with the lyophilized drug, resulting in a reconstituted drug solution. It is understood that as the piston rod 406 pushes the rear plug 38 forward, air in the front chamber 418 escapes through the needle 412. Continued movement of the piston rod 406 urges a rear plug 38b forward until it comes into contact with the front plug 38 and pushes a front plug 38a forward, closing off the channel 430 between the front and rear chambers 418,420. Then the back rod 408 continues to push the piston rod 406 and the combined plugs 38a and 38b, hereinafter the plug 38, to provide titration to the desired dosage (FIG. 54).

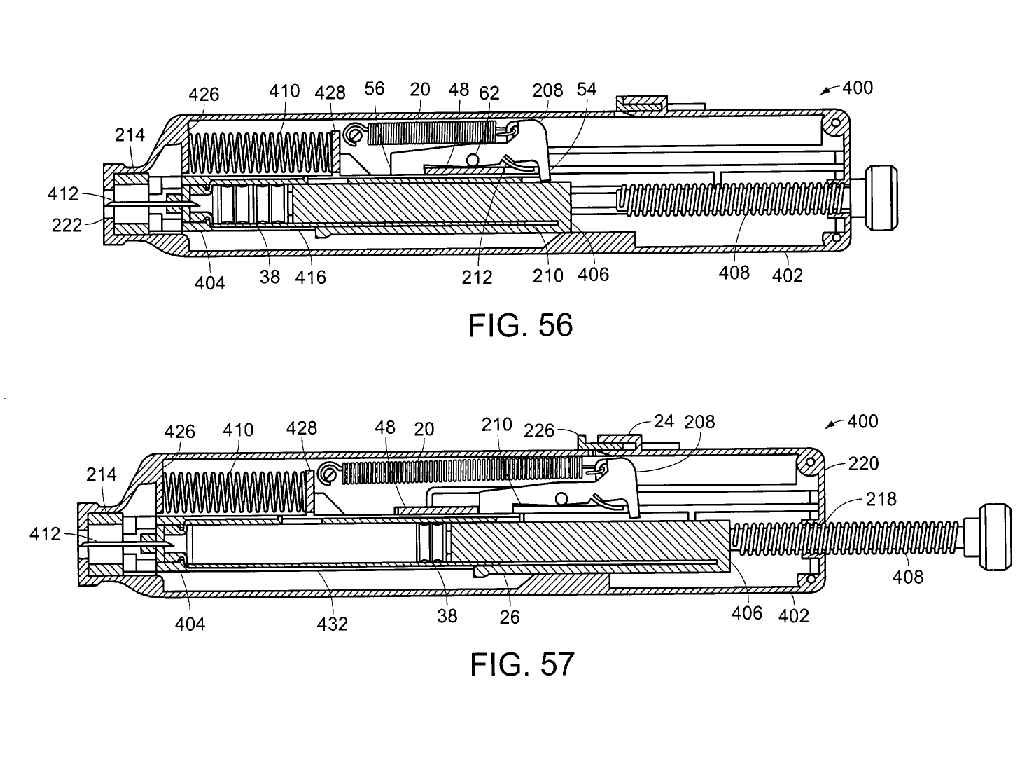

The injector 400 is now ready for drug delivery. As shown in FIGS. 54-56, the injector 400 is activated and delivers the drug as described in previous exemplary embodiments. That is, the safety tab 226 is pushed back into the trigger 24, the trigger 24 is pressed to pivot the control unit 208, allowing the driving unit 20 to pull the control unit 208 which moves the piston rod 406 through the cartridge 416 for delivery. At the end of delivery, the control unit 208 pivots to disengage the engaging finger 54 of the control unit 208 from the piston rod 406. This pivoting releases the driving unit 20 from the syringe 404, and allows the retracting unit (e.g., retracting spring 50) to push the needle 412 back into the housing 402, as shown in FIG. 56.

### Disposable Pre-Filled, Single Chamber Liquid Drug Cartridge

Another injector 400 constructed in accordance with an exemplary embodiment is shown in FIG. 57. The injector 400 is substantially similar to the injector 400 shown in FIG. 50, and uses a single-chamber cartridge 432 instead of the dual-chamber cartridge 416 discussed above. As shown in FIG. 57, the single-chamber cartridge 432 is prefilled with a liquid drug ready for an injection. Operation of the injector 400 using the single-chamber cartridge 432 is similar to that for the dual-chamber cartridge 416 and for brevity is not repeated here.

### Fully Disposable Pre-Filled Injector With Drug Titration

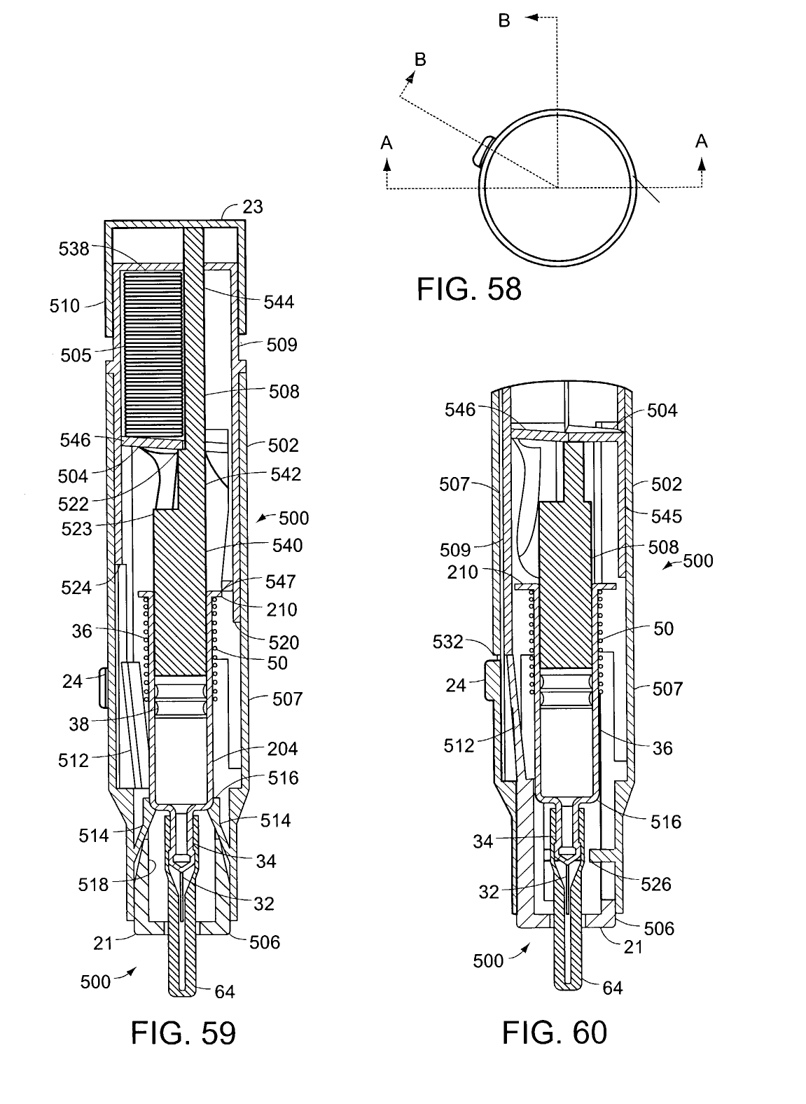

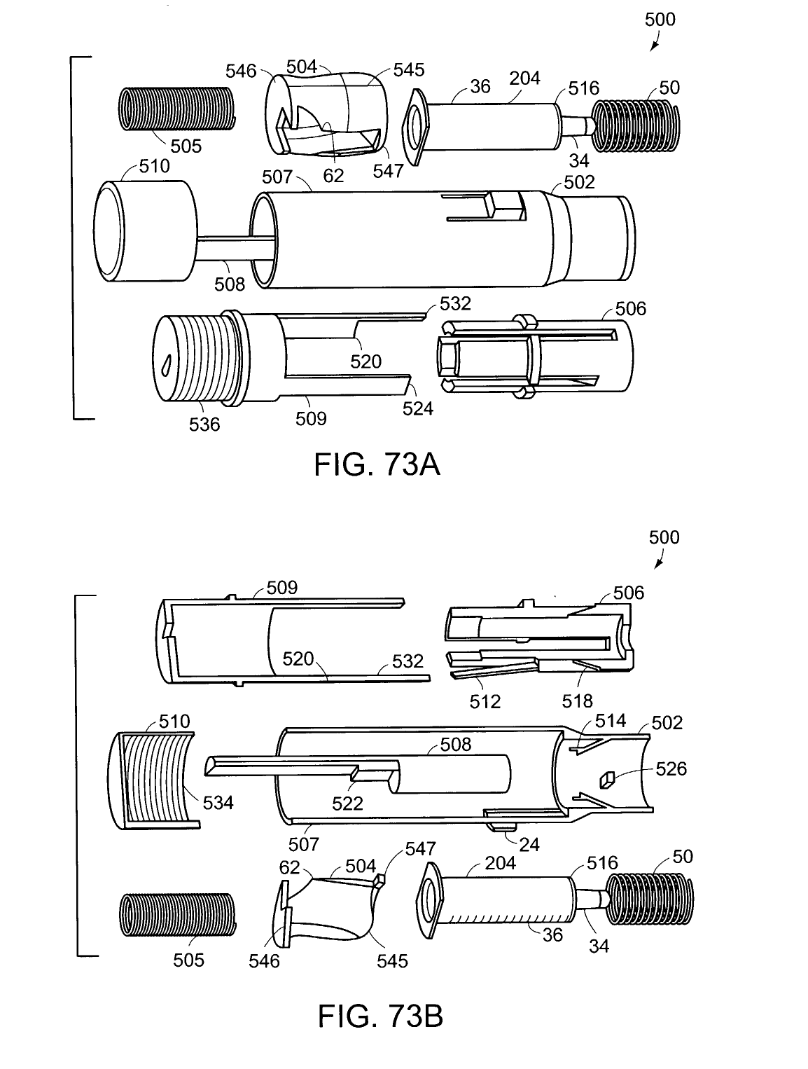

FIGS. 58-78 illustrates an injector 500 in accordance with yet still another exemplary embodiment of the invention. As can best be seen in FIGS. 73a and 73b, the injector 500 includes a housing 502, a syringe 204, a control unit 504, a driving unit 505, a body sensor 506, a retracting spring 50, a piston rod 508, a protective cap 64 (FIG. 59), and a handle 510. The housing 502 includes an outer housing 507 enclosing an inner housing 509, and is shaped to have its central axis coaxial with the needle axis. The syringe 204 includes a needle 32, a needle hub 34, a barrel 36, a plug 38 (FIG. 58), a radially extending flange 210 and a retaining shoulder 516. The housing 502 includes the outer housing 507 and the inner housing 509.

The driving unit 505 is a compression spring instead of an extension spring as discussed above. That is, the driving unit 505 comprises a spring that generates its driving force by expanding from an initial compressed state. Unlike the driving units 20 described above in the exemplary embodiments, e.g., spring 60 which requires its axial ends be coupled to the control unit 18 and the housing 12, respectively, (see FIG. 1), the driving unit 505 requires no such connections. During assembly, the driving unit 505 can simply be positioned in its proper location and then loaded into the compressed state. When released, the driving unit 505 pushes at its axial ends without requiring any connection of those axial ends to the internals of the injector 500. Furthermore, although the driving unit 505 has a central or longitudinal axis offset from the needle or syringe axis, the location of the driving unit 505 in the injector 500 can be closer to the axis of the injector 500 than the driving unit 20 of the embodiments discussed earlier. Thus, this makes the injector 500 more symmetrical with the central axis of the injector 500 being coaxial with the needle axis.

Referring to FIGS. 59 and 60, the driving unit 505 is located between a rear wall 538 of the inner housing 509 and the control unit 504, adjacent the piston rod 508. Like the exemplary embodiments described above, the driving unit 505 is eccentric to the injector 500, and not coaxial with the syringe 204. In other words, the driving unit 505 is offset from, but generally parallel to the control axis of the injector 500.

The piston rod 508 extends from the plug 38 in the barrel 36 through an opening in the rear wall 538 of the inner housing 509 to the handle 510. As shown in FIG. 59, the piston rod 508 preferably has three sections. The first section 540 extends from the plug 38 to a distal shoulder 523. The second section 542 extends from the distal shoulder 523 to a middle shoulder 522. The second section 542 has a cross-section narrower in width than the cross-section of the first section 540. The third section 544 extends from the middle shoulder 522 through the opening in the rear wall 538 to the handle 510. The third section 544 has a cross-section narrower in width than the cross-section of the second section 542.

The control unit 504 is arranged to slide and rotate within the housing 502. As can best be seen in FIG. 73a, the control unit 504 has a generally cylindrical shaped side wall 545 defining a top wall 546. The sidewall 545 and top wall 546 have a cutout arranged to allow the piston rod through the top wall 546. The cutout defines a lip 547 and a pivot axis 62. In operation, the control unit 504 is being tilted or pivoted twice within the housing 502, generally about the axis 62, to affect how the driving unit 505 acts upon the syringe 204 and the piston rod 508.

The retracting spring 50 coils about the syringe barrel 36 between the radially extending flange and the body sensor 506. The spring 50 automatically retracts the syringe 204, and thus retracts the needle 32 into the housing 502 after completion of drug delivery.

As shown in FIG. 59, the trigger 24 is located farther forward toward the distal end 21 than the triggers shown in the earlier described embodiments. As shown in FIGS. 59 and 60, the injector 500 also includes the body sensor 506 projecting from the distal end of the housing 502. When pressed against an injection site, the body sensor 506 slides into the housing 502 and, if activated by the trigger 24, releases the syringe 204. When the syringe 204 is released, it is available for injection and drug delivery, as described in greater detail below.

The body sensor 506 includes an activating latch 512 that abuts the trigger 24 to prevent premature axial movement of the body sensor 506 into the housing 502. Before activation of the injector 500, the activating latch 512 is arranged to extend out to the trigger 24 and next to a latch impeding shoulder 532 of the inner housing 509. As can be seen from the drawings, the activating latch 512 abuts the inner housing wall 509 which physically impedes any axial movement of the body sensor 506 toward the trigger 24. The activating latch 512 is located adjacent the trigger 24 and can be moved radially inward out of contact with the latch impeding shoulder 532 for activating the injector 500, as described in greater detail below.

The housing 502 also includes retaining latches 514 at the distal end 21 that extend radially inward and axially rearward to hold and secure the syringe 204 from prematurely moving axially forward or radially outward. The latches 514 are moved radially outward to allow drug delivery upon activation of the injector 500 as described in more detail below.

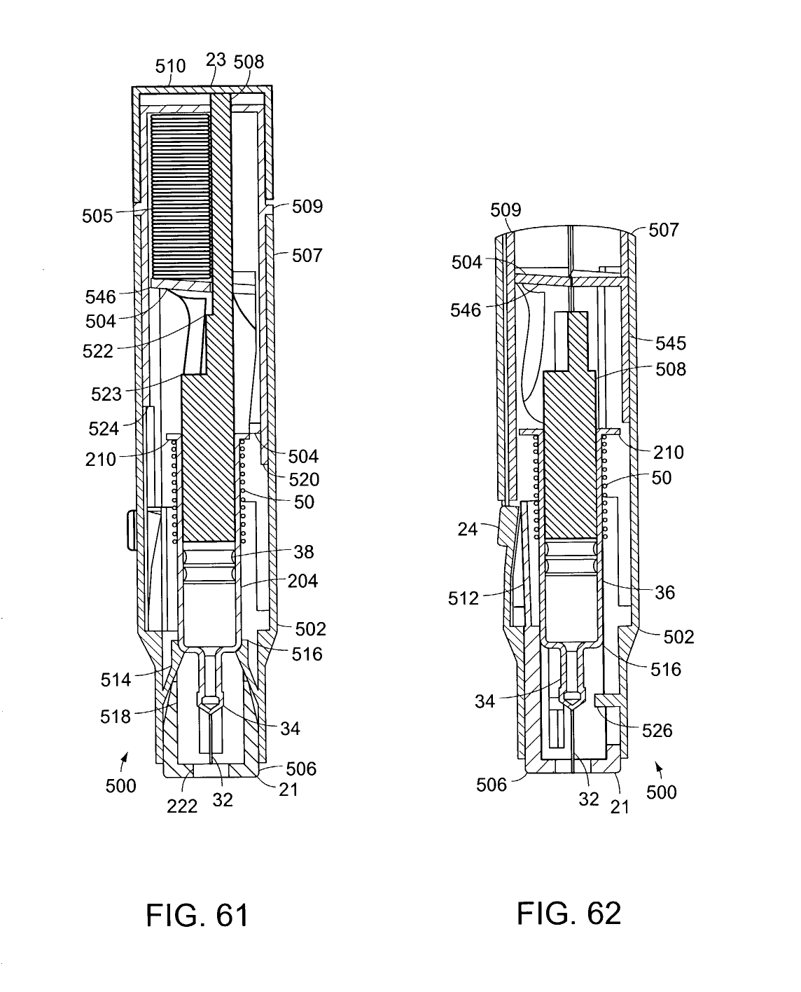

The first step in use of the injector 500 is to remove the sterility cap 64 from the needle 32. Titration can then be provided to obtain the desired dosage by holding the injector 500 vertically upward with the distal end 21 upright and above the proximate end 23, and rotating the handle 510. FIG. 61 shows the injector 500 after titration. While not being limited to a particular theory, the handle 510 has threads 534 (FIG. 73b) along its inner wall for engagement with matching threads 536 (FIG. 73a) on the adjacent exterior wall of the inner housing 509. Referring to FIG. 61, rotation of the handle 510 pushes the piston rod 508 into the plug 38 and pushes the unwanted volume of drug out the needle 32. This movement of the plug 38 also breaks static friction between the plug 38 and the syringe 204 so that the injection rate between the different units will be more consistent. Such static friction typically increases over time (e.g., storage) as the plug 38 does not move in the syringe 204. The titration, which is also provided in other exemplary embodiments discussed herein, also allows the syringe 204 to push out residual air bubbles remaining in the barrel 36.

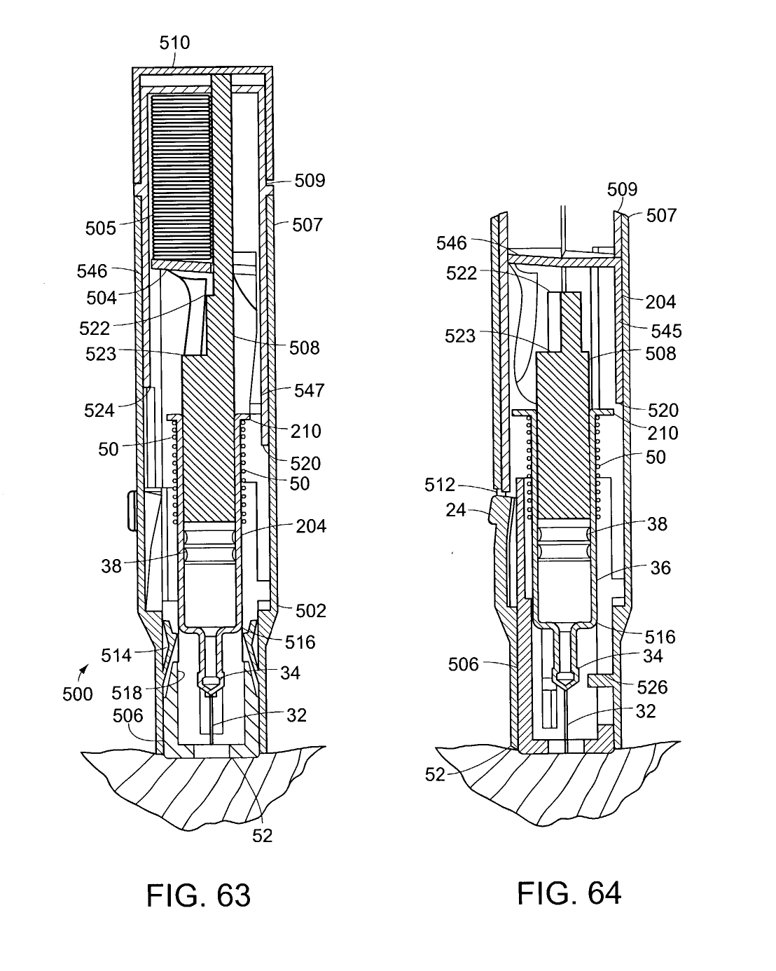

FIGS. 61 and 62 are sectional views taken along lines A-A and B-B of FIG. 58 after titration. These figures illustrate the safety features that allow needle penetration only if two conditions are fulfilled: pressing the front end of the body sensor 506 to the injection site 52, and depressing the trigger 24. As shown in FIGS. 61 and 62, if the trigger 24 is pushed to release the activating latch 512 without depressing the body sensor 506, injection will not occur. However, as shown in FIGS. 63 and 64, if the body sensor 506 is pressed against the injection site 52, and the trigger 24 is depressed, then the body sensor 506 pushes the latches 514 away from the retaining shoulder 516 of the syringe 204 and activates the injector 500.

The latches 514 are moved away from the syringe 204 as a tapered portion of the body sensor 506 moves toward the syringe 204 to abut and push the latches 514 outward, thereby disengaging the latches 514 from the syringe 204. The body sensor 506 cannot disengage the latches 514 from the syringe 204 until the trigger 24 pushes the activating latch 512 inward, releasing it from the latch impeding shoulder 532 of the housing 502.

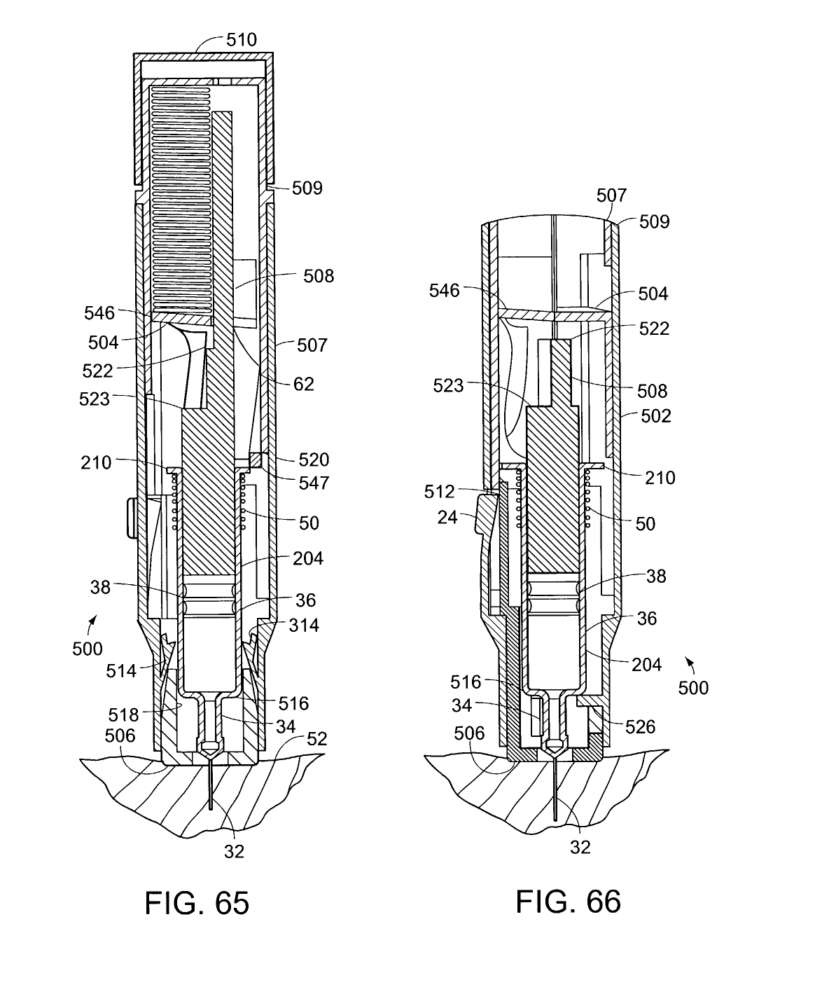

Once the latches 514 are moved away from and disengage from the syringe 204 (FIG. 63), the syringe is free to move toward the injection site by means of the driving unit 505. This enables the needle 32 to penetrate into the injection site 52. As shown in FIGS. 65 and 66, the control unit 504 is located along an inner wall of the inner housing 509 having its lip 547 in contact with the radially extending flange 210 of the syringe 204. The driving unit 505 pushes the top wall 546 of the control unit 504. This causes the lip 547 of the control unit 505 to act on and push the flange 210 of the syringe 204. This moves the syringe 204 toward the injection site, enabling the needle 32 to penetrate into the injection site 52, as shown in FIG. 65. This movement of the syringe 204 also presses the retracting spring 50 and holds the spring 50 in compression.

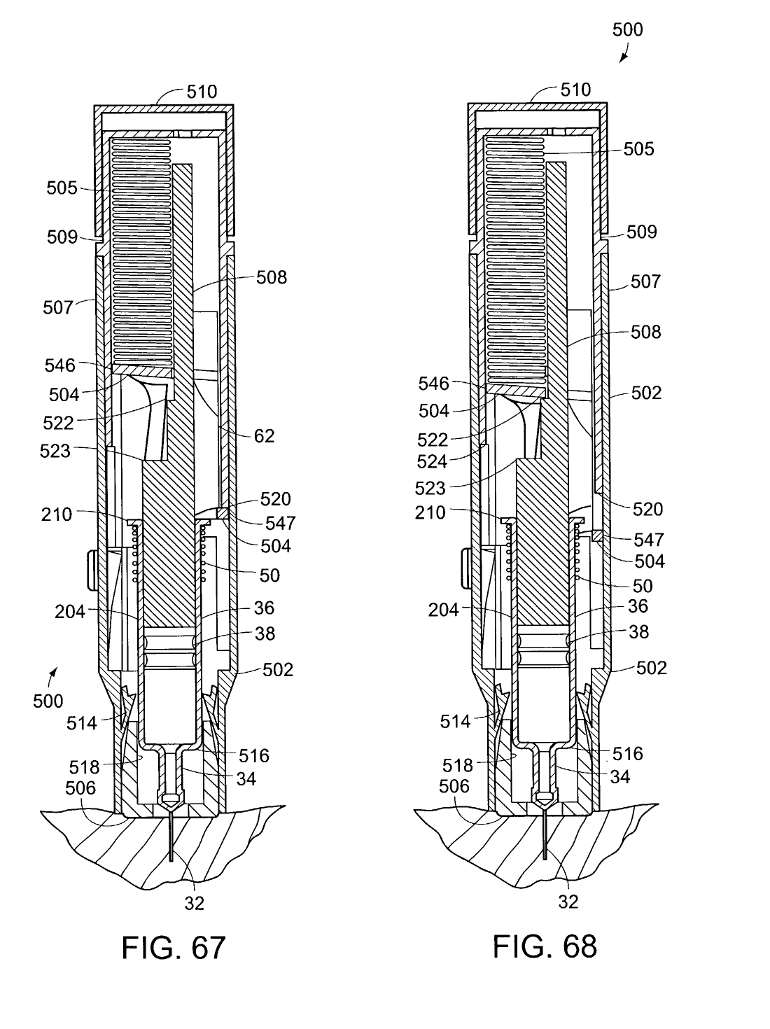

As can best be seen in FIGS. 65 and 67, the lip 547 of the control unit 504 reaches a first shoulder 520 of an inner wall of the housing. The continued push of the driving unit 505 on the top wall 546 eccentrically, applies a moment on the control unit 505 to tilt or pivot the control unit generally about its axis 62 once the lip 547 of the control unit 504 bypasses the shoulder 520. As the control unit 504 rotates, the lip 547 moves toward the outer housing 507 and separates from the radially extending flange 210 of the syringe 204. This separation releases the syringe 204 from the bias of the driving unit 505.

The piston rod 508 and syringe 204 are momentarily free to be moved backward by the retracting spring 50. However, any rearward movement at this time is slight as the driving unit 505 pushes the control unit 504 forward and the control unit 504 abuts the middle shoulder 522 of the piston rod 508, as shown in FIG. 68. The driving unit 505 pushes the control unit 504 against the middle shoulder 522, thereby moving the piston rod 508 into the barrel 36 to deliver the drug through the needle 32.

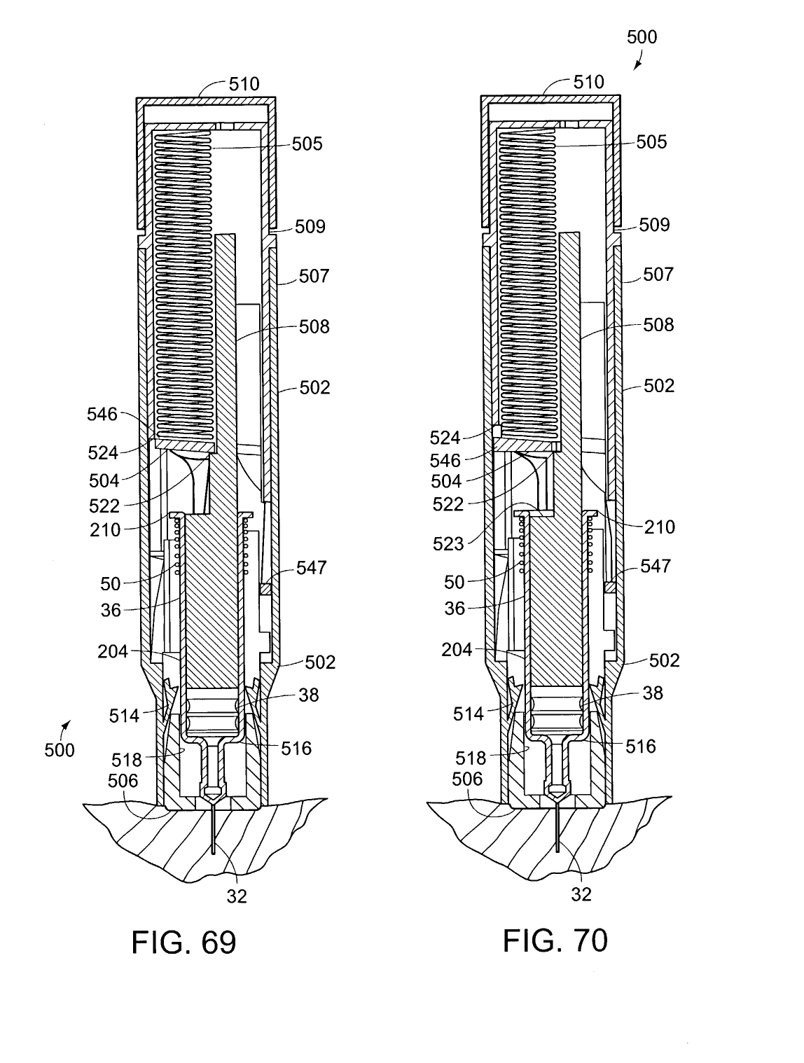

FIG. 69 shows the injector 500 at the end of delivery. As shown, the driving unit 505 has pushed the piston rod 508 through the barrel 36 so that the plug 38 is at the bottom of the barrel 36. The flexibility of an inwardly extending stop latches 526 (FIG. 60) helps to overcompensate for any over tolerances, to insure full delivery. That is, once all the driving spring reaction is static and on the stop latches 526, the force is deflect enough to compensate over tolerances. The control unit 504 bypasses a second shoulder 524 of the inner housing 507. This causes the control unit 504 to be tilted or pivoted again, and to move the top wall 546 towards the outer housing 507. The top wall 546 moves radially toward the outer housing 507, and separates from the middle shoulder 522 of the piston rod 508, as shown in FIG. 70. This separation releases the piston rod 508 from the bias of the driving unit 505. Again, this rotation is the result of moment caused by the force of expansion of the driving unit 505 and the eccentricity of its location.

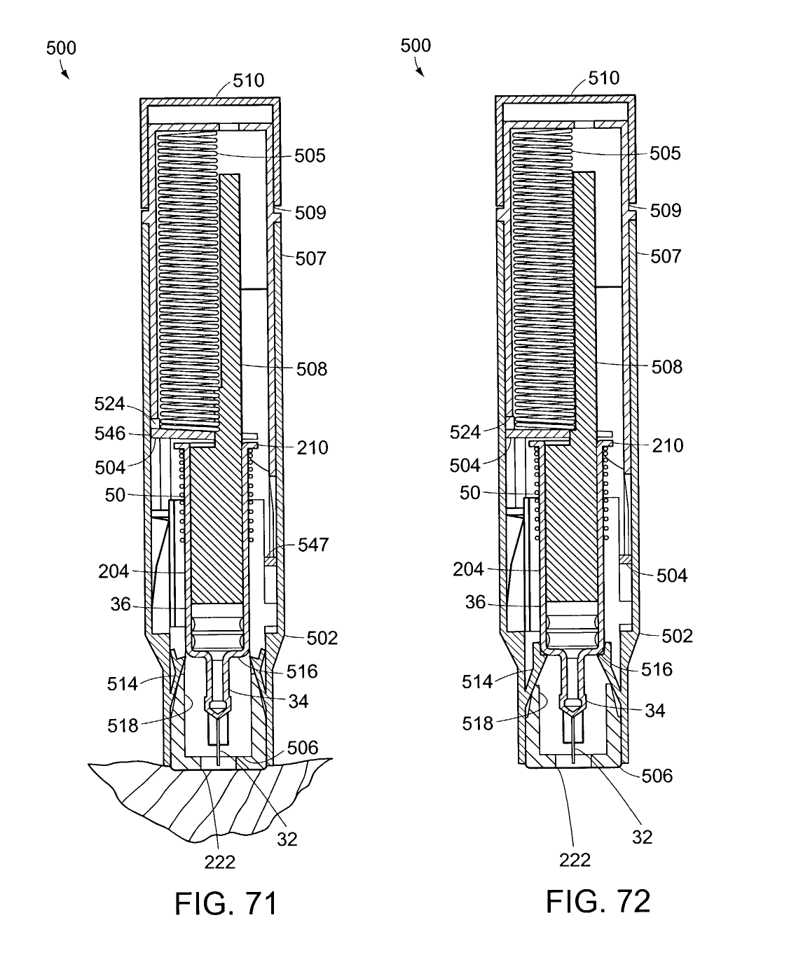

As shown in FIG. 70, the control unit 504 pivots and disengages from the middle shoulder 522 of the piston rod 508. This pivot releases the syringe 204 from the driving unit 505 and allows the retracting spring 50 to push the needle 32 back into the housing 502, as shown in FIG. 71. When the syringe 204 is pushed back to its pre-activation position, the latches 514 are allowed to move radially inward and again engage the syringe 204 (FIG. 72). The latches 514 therefore lock the syringe 204 and needle 32 from re-exposure.

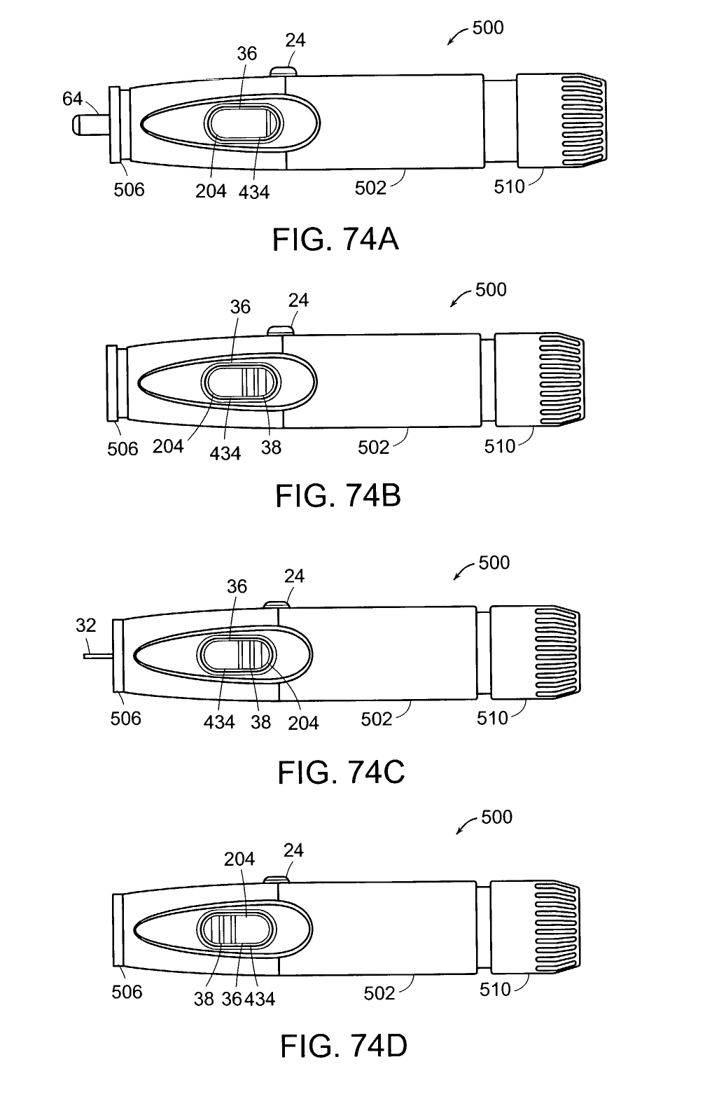

The housing 502 of this exemplary embodiment preferably includes a window 434 that allows a user to view the contents and amount of dosage in the syringe 204 before, during and after delivery. FIGS. 74a-74d are side views of the injector 500 corresponding to the injector 500 at the stages shown in FIGS. 59, 61, 65 and 71, respectively. That is, FIG. 74a shows the injector 500 during storage, FIG. 74b shows the injector 500 during titration, FIG. 74c shows the injector 500 during injection, and FIG. 74d shows the injector 500 after delivery.

A user looking through the window 434 of injector 500 can observe the amount of dosage in the syringe 204. During storage (FIG. 74a), the syringe 204 is filled with the drug solution. During titration, extra solution and air bubbles are pushed out of the barrel 36. As shown in FIG. 74b, the amount of solution is less than the amount shown in FIG. 74a, and can be easily measured by a user. During injection, the syringe 204 is moved to extend the needle 32 out of the housing 502, as shown in FIG. 74c. This movement is observable to a user looking in the window 434 as the barrel 36 and plug 38 move with the syringe 204 within the housing 502. After delivery, the barrel 36 is empty, as shown in FIG. 74d. The user knows the drug has been delivered by viewing the plug 38 located at the bottom of the barrel 36. Accordingly, the user or patient can easily view the dosage for delivery prior, during and after use.

### Fully Disposable Pre-Filled Injector With Drug Titration and Improved Trigger Assembly

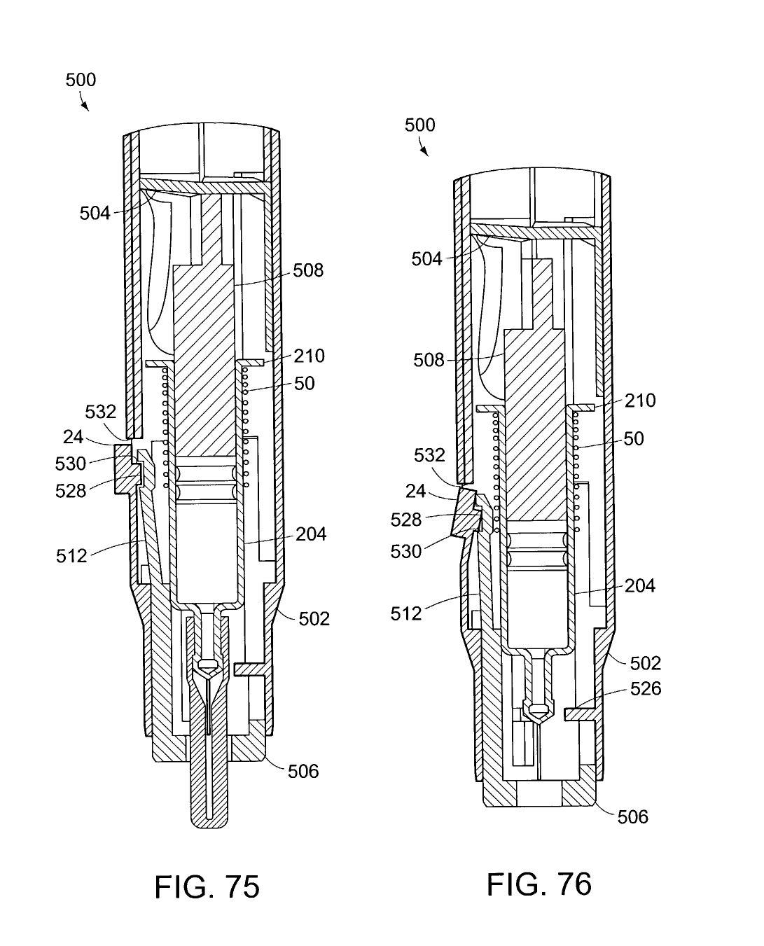

A preferred trigger assembly available with the injector 500 shown in FIGS. 58 through 72 is illustrated by example in FIGS. 75-78. As noted above, activating the injector 500 requires two conditions: attachment to the injection site 52 and depression of the trigger 24. If one of these two conditions is not met, injection does not occur. The example shown in FIGS. 75-78 improves upon the above-discussed injector 500 by requiring a specific order of sequences. First, the injector 500 must be pressed against the injection site 52, and only then will depressing the trigger 24 activate the injector 500. If the trigger 24 is held in its depressed position before the injector 500 is attached to the injection site 52, the injector 500 will not activate. This safety feature therefore prevents premature activation if the trigger 24 is depressed and then the injector 500 pressed against an undesired object or area of the patient.

FIG. 75 illustrates the improved trigger assembly. Here, the trigger 24 includes a projection 528 extending radially inward toward the activating latch 512. In addition, the activating latch 512 includes a notch 530 aligned with the projection 528 when the body sensor 506 is in its fully extended position. The body sensor 506 is in this fully extended position before the injector 500 is pressed against an injection site 52.

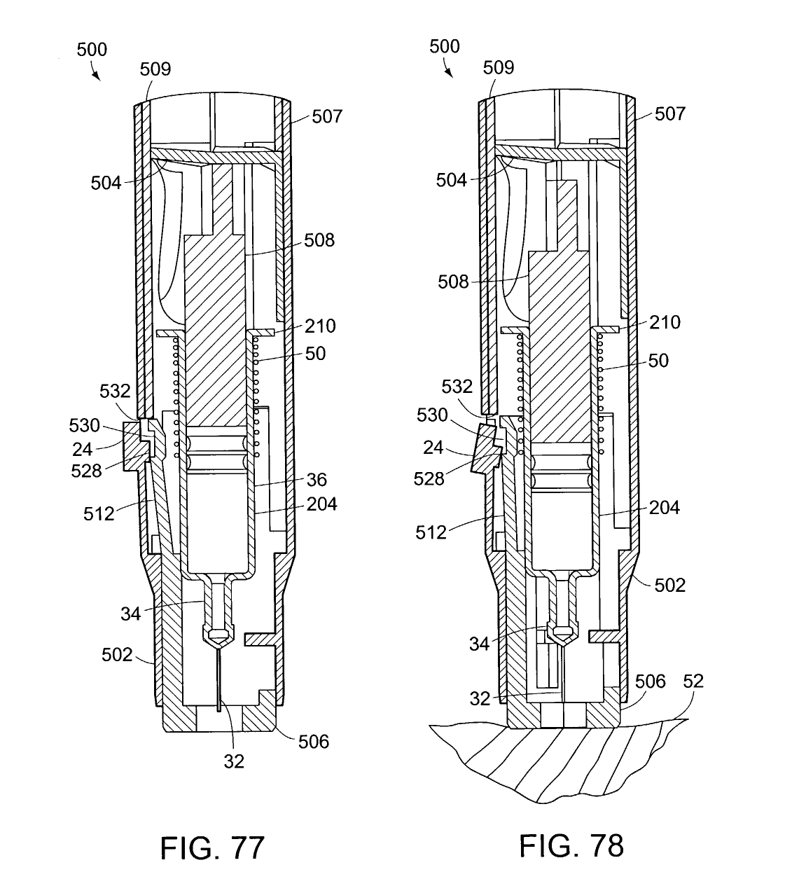

As shown if FIG. 76, pressing on the trigger 24 without depression of the body sensor 506 to the injection site 52 will not allow later activation until the trigger 24 is released. That is, if the trigger 24 is pushed first, the projection 528 of the trigger 24 is received into the notch 530 of the activating latch 512 and prevents axial movement of the latch 512, thereby preventing delivery. However, as can best be seen in FIG. 77, if the body sensor 506 is depressed against the injection site 52 before the trigger 24 is depressed, the body sensor 506 moves toward the syringe 204 a first increment until the activating latch 512 abuts the latch impeding shoulder 532 of the housing 502. This incremental movement of the body sensor 506 moves the notch 530 of the activating latch 512 out of alignment with the trigger projection 528. Accordingly, as shown in FIG. 78, when the trigger 24 is depressed, the projection 528 pushes against the activating latch 512. Since the trigger projection 528 and the activating latch 512 are not in alignment, the body sensor 506 can move a second increment, which disengages the housing latches 514 from the syringe 204 (FIG. 63), and frees the driving unit 505 and the syringe 204 for injection, delivery and automatic needle retraction, as discussed above regarding FIGS. 65-72.

### Concentric Disposable Injector With Drug Titration

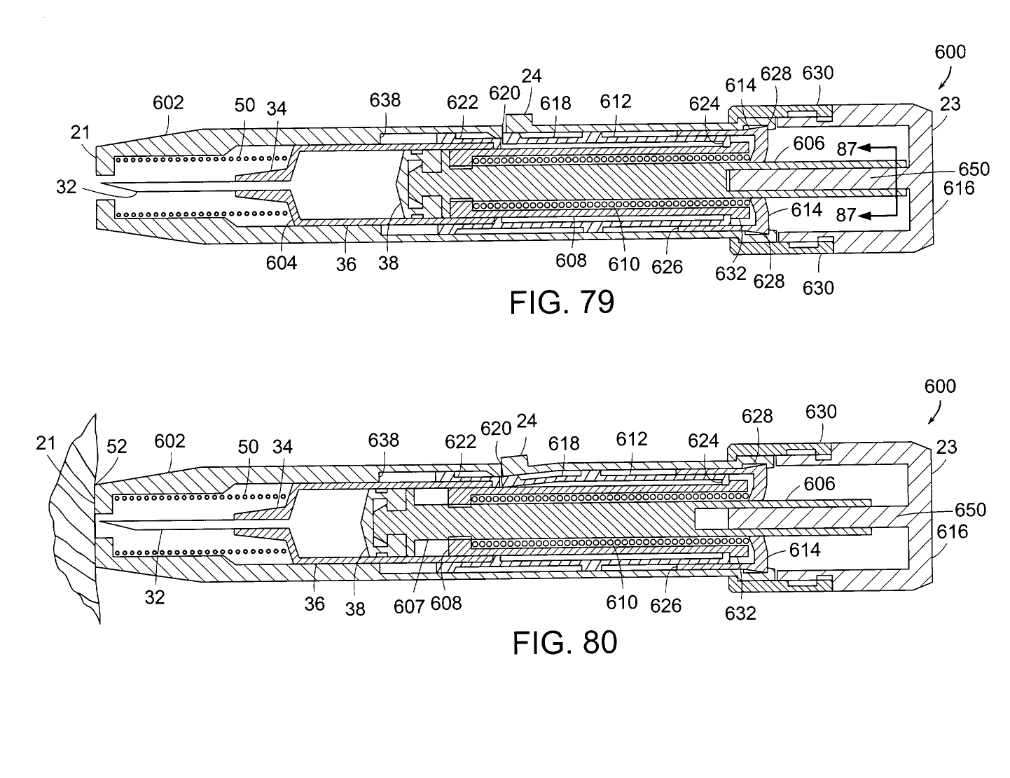

Referring to FIGS. 79-87 there is shown at 600 an injector constructed in accordance with yet another exemplary embodiment of this invention. The injector 600 includes a housing 602, a prefilled syringe 604, a piston rod 606, a control unit 608, a driving unit 610, a trigger 24, an external plunger 612, a rear support 614, a rear cover 616, a retracting spring 50, and a plug 38.

The housing 602 has a distal end 21 and a proximate end 23. The rear cover 616 attaches to the housing 602 in a rotational relationship. That is, the rear cover 616 is arranged to rotate at the proximate end 23 against the housing 602. The rear cover 616 includes a rear cover adaptor 630 that communicates, for example, by snap fitting with the housing 602 to keep the rear cover 616 rotationally aligned and coupled to the housing 602. The syringe 604 includes the needle 32, the needle hub 34, the barrel 36 and the plug 38.

In this embodiment, the control unit 608 is a sleeve or tube shaped member located around the driving unit 610. As shown in FIGS. 79 and 80, the control unit 608 is arranged to extend to and abut the rear support 614 at one of its longitudinal ends, and is arranged with threads at its other longitudinal end to engage matching threads along the driving rod 606 at section 607 of the driving rod. This threaded arrangement allows the driving rod 606 to rotate and move forward of the control unit 608 for titration, as will be described in greater detail below.

The driving unit 610 is a compression spring located about the piston rod 606 between the control unit 608 and the rear support 614. The driving unit 610 pushes against the rear support 614 and the control unit 608 to push the piston rod 606 forward through the prefilled syringe 604 for delivering the drug as described in greater detail below. Without being limited to a particular theory, the injector 600, shown in FIGS. 79-87, is cylindrical and the needle 32, syringe 604, driving unit 610 and retracting spring 50 are all coaxial. In other words, the needle 32, syringe 604, driving unit 610 and retracting spring 50 all have the same longitudinal axis.

The rear support 614 is preferably made of a flexible resilient material, such as rubber or plastic. The rear support 614 is shaped as a flexible washer 614 having an interior diameter less than a diameter of the piston rod 606 so that the rear support 614 is arranged to be forced radially outward when communicating with the piston rod 606, as shown in FIGS. 79 and 80. In fact, the rear support 614 is pushed outwards into engagement with rear notch 628 of the housing 602, which holds the rear support from axial movement, as shown in FIG. 79. The rear support 614 has a support sleeve 626 that extends inside the housing 602 towards the syringe 604.

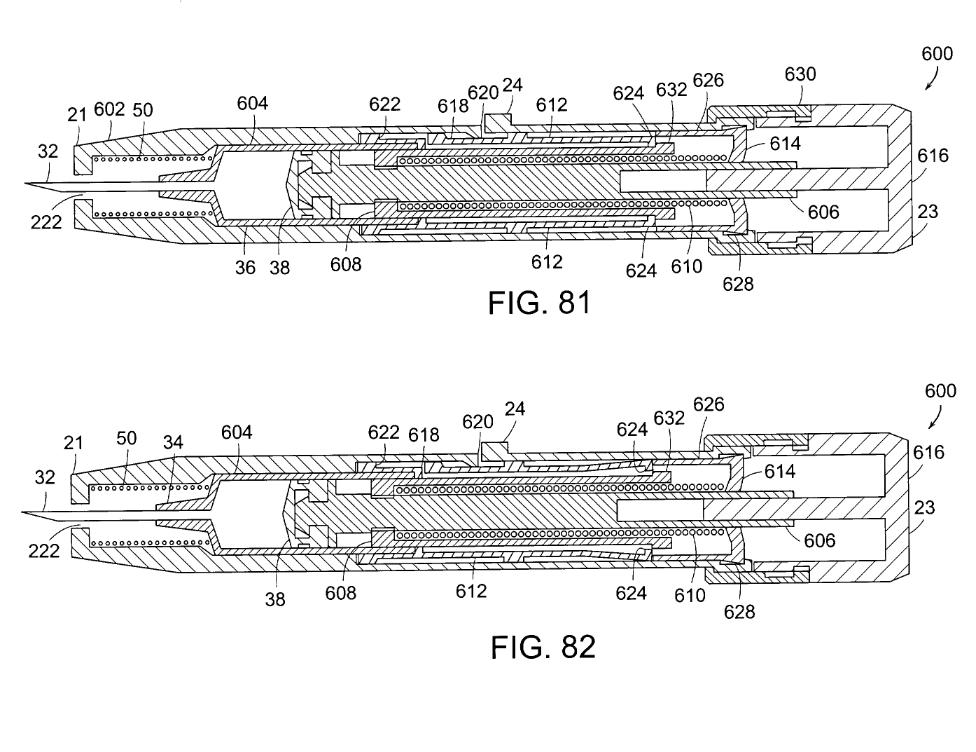

The external plunger 612 is generally sleeve or tube shaped and resilient. It is positioned within the housing 602 and about the barrel 38 and the control unit 608. Without being limited to a particular theory, the external plunger 612 aids in the injection and delivery of the drug. The external plunger 612 includes penetration-releasing latch 624 that prevents an premature delivery of the drug, as described for example below. The external plunger 612 also includes an activating latch 618, which abuts shoulder 612 of the housing until the injector is activated by the trigger 24. In addition, the external plunger 612 includes a penetration latch 622 located about the barrel. The penetration latch 622 is arranged to move the syringe 604 toward the distal end 21 for injection. Forward movement of the penetration latch is restricted by a shoulder 638 of the housing 602 (FIG. 81).

### Titration

The injector 600 provides titration by rotation of the rear cover 616, which also releases static friction between the plug 38 and the barrel 36. The cover 616 includes a back rod 650 slidingly engaged in the piston rod 606. FIG. 87 is a cross-sectional view of the piston rod 606 and the back rod 650. While the piston rod 606 preferably has a circular outer wall in cross section, an interior channel 648 of the piston rod is not circular. For example, as best seen in FIG. 87, the interior channel 648 is a polygon (e.g., square), defining the shape of the back rod 650. The interior channel 648 is preferably non-circular so that the piston rod 606 will rotate with rotation of the back rod 650. Therefore the piston rod 606 rotates with rotation of the rear cover 616.

It is understood that the invention is not limited to a particular shape. Accordingly, it is within the scope of the invention that the cross-sectional shape of the interior channel 648 and the back rod 650 have any shape that allows the back rod 650 to slide within the interior channel 648 and cause the piston rod 606 to rotate when the rear cover 616 is rotated. Moreover, the interior channel 648 and back rod 650 are not required to have the same shape, as long as the rotation of the back rod 650 turns the piston rod 606. For example, the cross-sectional shape of the interior channel 648 could be square, and the cross-sectional shape of any part of the back rod 650 that is positioned within the channel could be X-shaped extending to the corners of the square interior channel.

As noted above, the control unit 608 and rod 606 are threaded to allow axial movement of the piston rod 606 upon rotation of the rear cover 616. Rotating the rear cover 616 causes the rotation of the piston rod 606, which moves the piston rod 606 axially relative to the control unit 608 towards the plug 38 and pushes the plug through the housing 602 of the syringe 604. Accordingly, the rotation movement of the rear cover 616 results in axial movement of the piston rod 606 which pushes the drug or air in the cartridge/syringe out of the needle and allows drug titration.

### Drug Delivery

After titration, the injector 600 is ready for use. As shown in FIG. 80, the injector 600 is placed against the injection site 52. The trigger 24 is pressed radially inward to move the activating latch 618 of the external plunger 612, which is blocked from moving forward during shelf life by the latch bumper 620 of the housing 602. The activating latch 618 moves radially inward separating from the latch bumper 620 which allows axial movement of the external plunger, and permits expansion of the driving unit 610. The driving unit 610 pushes the control unit 608 which moves the penetration releasing latch 624, and thus, the external plunger 612 forward, pushing the syringe 604 forward for needle penetration. The control unit 608 pushes the external plunger 612 until the penetration releasing latch 624 slides forward or distal of the rear support sleeve 626. As shown in FIG. 81, the latch 624 is allowed to flip and extends radially outward to the housing 602, disengaging from the rear support sleeve 626 as shown in FIG. 82. The penetration-releasing latch 624 is flexible and designed to expand outward unless it is supported by the rear support sleeve 626.

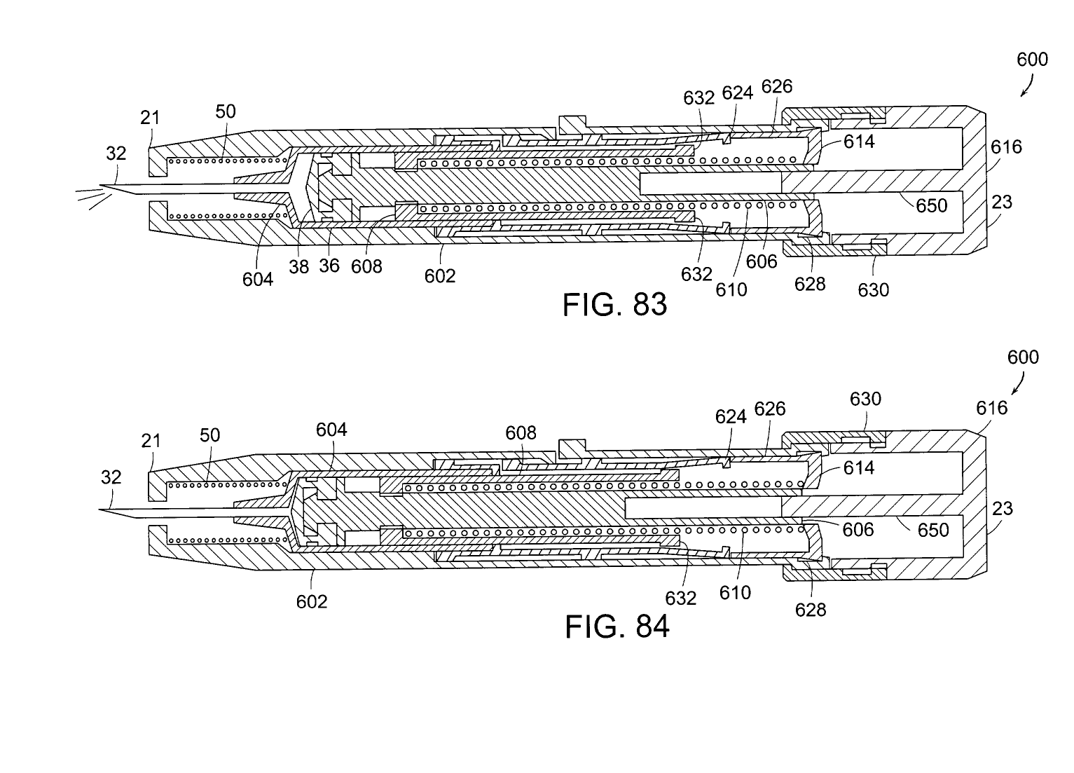

As the penetration releasing latch 624 moves outward, it releases the external plunger 612. The driving unit 610 continues to expand and pushes the control unit 608. Because the control unit 608 is threadingly engaged with the piston rod, the control unit moves the piston rod 606 through the syringe barrel 36 and delivers the drug from the barrel, as shown in FIG. 83.As the piston rod 606 moves forward, it slides relative to the back rod 650. This relationship is best seen in FIGS. 79 through 84.

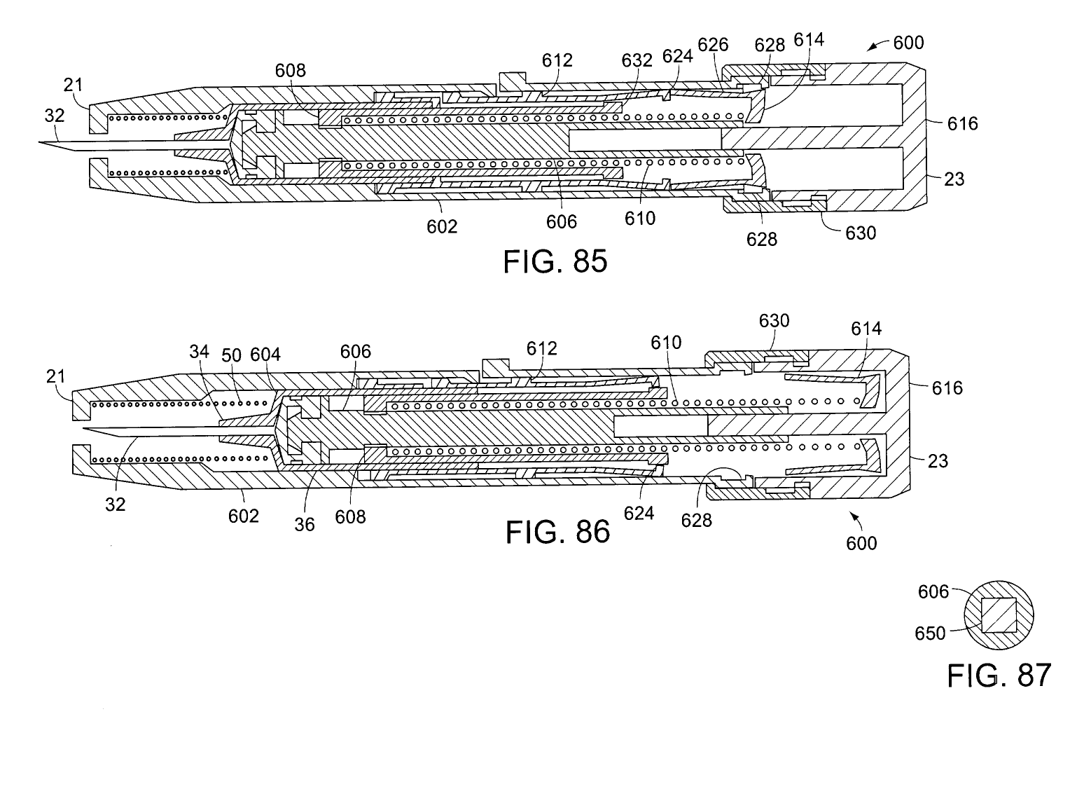

Upon the end of delivery (FIG. 84), the piston rod 606 slides forward far enough that it becomes detached from the rear support 614. That is, the piston rod 606 automatically separates from the rear support 614. Once the rear support 614 is separated from the piston rod 606, the rear support 614 is allowed and arranged to move radially inward towards the back rod 650 because of its flexibility. This movement releases the rear support 614 from the rear notch 628 of the housing 602, as shown in FIG. 85. This release disengages the rear support 614 from the housing 602 and thus releases the hold of the rear support on the driving unit 610. Thereafter, as shown in FIG. 86, the driving unit 610 pushes the rear support 614 toward the cover 616. Further, the release of the rear support 614 from the housing 602 releases a hold on the syringe 604, thus allowing axial movement of the syringe away from the distal end, for retraction of the needle 32.

The retracting spring 50 is arranged to push the syringe 604 rearward upon the release of the syringe 604 from the rear support 614 and retract the needle 32 into the housing 602. As long as the driving unit 610 is not longer biasing the control unit 608, and thus, the syringe 604, the needle 32 is not re-exposed as the retracting spring 50 prevents forward movement of the needle 32 out of the housing 602.

The injectors constructed in accordance with the exemplary embodiments provide a safe and efficient approach to delivering a drug into a patient. The injector can be used as a disposable device or a re-usable device and can incorporate various combinations of the features described herein. For example, as a disposable device, the injector can be packaged at least as a prefilled syringe, or as an empty device having a vial adapter for filling the syringe with a drug from a vial. The injector can be packaged as a re-usable device including a disposable cartridge with a prefilled syringe, or a cartridge with an empty syringe and vial adapter. The cartridges can contain a liquid drug or a lyophilized drug that is reconstituted using the injector, with a diluent for delivery.

The exemplary embodiments show each injector having a distal end from which the needle is exposed, and a proximate end opposite the distal end. In the exemplary embodiments, the injector automatically deploys its needle, delivers the drug in the syringe and retracts the needle back into the housing. Preferably the injector provides a distinct end of delivery indication (e.g., a ‘click-type’ effect and associated impaction). The injector can be constructed, for example, with the driving unit eccentric to the syringe, a retracting spring eccentric to the syringe, or with the driving unit and the retracting spring eccentric to the syringe. The injector can be assembled around a syringe or assembled for use with a syringe assembly having a cartridge inserted through the distal end of the injector or through a side opening in the injector housing. The barrel of the syringe can be empty or prefilled, and can include a cartridge having a single chamber or a multiple chamber. As a further feature of some exemplary embodiments, the syringe includes a piston rod that can be moved within the syringe barrel for titration, reconstitution or filling by a linear back rod, a threaded back rod, or a ratchet. The piston rod can be moved in one direction only or in both directions, as desired.

The injector provides various safety features for minimizing exposure of the needle. These features include false activating prevention mechanisms, body sensing mechanisms and needle-locking mechanisms for locking the needle before and/or after use. The injector optionally includes damping material (e.g., the bushing, shock absorbing tab) for shock noise reduction. The bushing also minimizes residual drug spillage. The injector provides linear rate control using a low elasticity constant spring, preferably in the form of an expansion spring. The expansion spring can be made longer so that the syringe and piston rod move over a small longitudinal range compared to the length of the spring, thereby allowing the force of the spring to be consistent over the smaller range. In addition, by keeping the needle inside the housing before injection, the injector provides titration clearances preventing effects of shocks over the loss of drug out of the needle during titration.

The delivery devices of the exemplary embodiments allow for accurate titration and measurement of the amount of drug compound to be injected and provide an approach for fixing mistakes or overdose of air bubbles. A skilled artisan can readily understand that this approach also allows for the mixing and reconstitution of drugs using multiple vials or ampoules for the same injection, as vials and ampoules can be replaced while the drug solution is locked within the barrel of the syringe. Thus, drugs can be readily mixed and/or reconstituted to a desired dosage for delivery. Moreover, since the end of delivery is clear, no eye contact is required for indication of the end of delivery, thus making the delivery easier when the user cannot see the injection area.

As a person skilled in the art would readily understand, delivery of the fluid drug is determined not only by the driving unit or spring. It also depends on fluid properties and the fluid's path geometry. Therefore, delivery curves will not be identical to spring reaction curves. The fluid acts as a hydraulic damper and its resistance to flow is related to the force applied to it.

The driving unit in the exemplary embodiments can be either a compression spring or an extension spring. The compression spring is preferably used in the embodiments having a substantially symmetrical housing cross-section, and the extension spring can be seen in embodiments having a substantially oval housing cross-section. An extension spring need spare coils at the ends to fix the spring to the housing and control unit. Extension springs provide the benefit of allowing preloading—internal force in the spring even before it is extended. This is achieved by applying torsion forces to the spring during production. The result is the ability to have a loaded spring without significant added length. Compression springs cannot be preloaded during production. At best, preloading can be achieved by initial compression. The required motion range and the accumulated thickness of the coils limit this initial compression.

The driving spring is the most available element to control delivery. The main feature provided from the spring is a low elasticity constant. A low constant provides a more uniformed delivery profile, more flexibility in controlling delivery duration, spring load reduction during shelf life, and it provides sufficient force at the end of the injection cycle. Some of the exemplary injectors have eccentric driving units that are extension springs. These injectors allow use of a long spring relative to the size of the device. Using long springs provides the benefit of improving delivery time control and profile by changing the spring's constant of elasticity and by allowing preloads.

Moreover, this invention overcomes other problems associated with the prior art. For example, the control unit and retraction springs overcome the problems of needle phobia and needle injury. In addition, the injectors include a back rod and a piston rod that provides the advantage of titration and reconstitution in an automatic injector to allow a patient to measure and self administer a dosage via an automatic injection system, with the back rod automatically separating from the piston rod before delivery. The injectors with a back rod also enable the user to minimize residual drug in the system and to eliminate air bubbles that may otherwise be trapped in the automatic system prior to use. Further, the window provides the user with the ability to see dosage formulation prior to use, and to see that the drug has been delivered after use.

It should be apparent from the aforementioned description and attached drawings that the concept of the present application may be readily applied to a variety of preferred embodiments, including the exemplary embodiments disclosed herein. For example, other driving and retraction units, such as elastomeric “O” rings or compressed gas may be used in place of the extension and compression springs disclosed herein to bias the control unit, piston rod and syringe, as readily understood by a skilled artisan. In addition, it is understood that an ampoule adapter could be used instead of the vial adapter shown in the drawing when the drug is contained in an ampoule instead of a vial.

It is further appreciated that the present invention may be used to deliver a number of drugs. The term “drug” used herein includes but is not limited to peptides or proteins (and mimetic thereof), antigens, vaccines, including DNA vaccines, hormones, analgesics, anti-migraine agents, anti-coagulant agents, medications directed to the treatment of diseases and conditions of the central nervous system, narcotic antagonists, immunosuppressants, agents used in the treatment of AIDS, chelating agents, anti-anginal agents, chemotherapy agents, sedatives, anti-neoplastics, prostaglandins, antidiuretic agents and DNA or DNA/RNA molecules to support gene therapy.

Typical drugs include peptides, proteins or hormones (or any memetic or analogues of any thereof) such as insulin, calcitonin, calcitonin gene regulating protein, atrial natriuretic protein, colony stimulating factor, betaseron, erythropoietin (EPO), interferons such as α, β, or γ interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra, thereof; analgesics such as fentanyl, sufentanil, butorphanol, bup renorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; antiemetic agents such as scopolamine, ondansetron, domperidone, metoclopramide, and analogues thereof; cardiovascular agents, anti-hypertensive agents and vasodilators such as diltiazem, clonidine, nifedipine, verapamnil, isosorbide-5-mononitrate, organic nitrates, agents used in treatment of heart disorders, and analogues thereof; sedatives such as benzodiazepines, phenothiozines, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluoxamine, Bisolperol, tactolimuls, sacrolimus and cyclosporin.

Without further elaboration, the foregoing will so fully illustrate the invention that others may, by applying current or future knowledge, readily adapt the same for use under various conditions of service.

What is claimed is:

1. An automatic injector for delivering a fluid, comprising:
a housing including an activator arranged to activate said injector, said housing having a proximate end and a distal end;
a syringe positioned within said housing, said syringe having a needle extending towards said distal end, a barrel, and a piston rod, said barrel arranged to contain the fluid therein, the fluid in communication with said needle, said piston rod slidingly located within said barrel for forcing the fluid through said needle upon activation of said injector;
a control unit adapted to engage said piston rod, said control unit further adapted to slide and pivot within said housing; and
a driving unit in communication with said housing and in communication with said control unit, said driving unit arranged to bias said control unit causing said control unit to slide towards said distal end and move said piston rod through said barrel to push the fluid through said needle for delivery into an injection site, said control unit arranged to pivot out of engagement with said piston rod at the end of delivery.

2. The automatic injector of claim 1, wherein said syringe has a first longitudinal axis, and said driving unit has a second longitudinal axis different than said first longitudinal axis.

3. The automatic injector of claim 2, wherein said second longitudinal axis is substantially parallel to said first longitudinal axis.

4. The automatic injector of claim 2, further comprising an automatic retracting mechanism that automatically retracts said needle into said housing after the rotation of the control unit.

5. The automatic injector of claim 4, said housing including a set of latches extending longitudinally rearward towards said proximate end and extending radially inward, said latches adapted to abut said syringe and prevent axial movement of said syringe toward said distal end of the housing after retraction of said needle.

6. The automatic injector of claim 4, wherein said retracting mechanism comprises a spring having a third longitudinal axis different from said first longitudinal axis.

7. The automatic injector of claim 4, said retracting mechanism comprising a spring adapted to provide a pushing force, said driving unit adapted to provide a pulling force greater than said pushing force.

8. The automatic injector of claim 3, said control unit further comprising a proximate end and a leaf spring extending towards said proximate end, said leaf spring arranged to abut said syringe after retraction of said needle and prevent potential re-exposure of said needle.

9. The automatic injector of claim 1, wherein said driving unit is a spring arranged to bias said control unit to pull said piston rod into said barrel and then move said control unit into disengagement from said piston rod.

10. The automatic injector of claim 1, said piston rod including a holding latch, said holding latch arranged to engage said housing prior to an activation of said injector, said holding latch arranged to disengage said housing and engage said syringe to move said needle to the injection site upon activation of said injector, and said holding latch arranged to disengage said syringe upon movement of said needle to the injection site.

11. The automatic injector of claim 1, said control unit arranged to pivot to engage said control unit with said piston rod before delivery.

12. The automatic injector of claim 1, said piston rod including a holding latch that releasably engages both said housing and said syringe to bias said needle from said housing for injection of the fluid.

13. The automatic injector of claim 1, said housing including a cartridge, said cartridge releasably connected within said housing and adapted to hold said barrel.

14. The automatic injector of claim 1, said housing having an opening at said proximate end, said injector further comprising a back rod extending through said opening and arranged to push said piston rod into said barrel before activation of said injector.

15. The automatic injector of claim 14, said back rod having a threaded edge for rotational axial movement m relation to said opening.

16. The automatic injector of claim 14, said back rod having a serrated edge for incremental axial movement in relation to said opening.

17. The automatic injector of claim 16, further comprising a pawl for engagement with said serrated edge, said pawl arranged to allow incremental movement of said back rod towards said piston rod and prevent movement of said back rod away from said piston rod.

18. The automatic injector of claim 14, said back rod arranged to pull said piston to draw the fluid or a liquid into said barrel.

19. The automatic injector of claim 14, said back rod adapted to pivot about said opening to attach to said piston rod, said attachment allowing said back rod to push said piston rod into said barrel and to pull said piston rod from said barrel.

20. The automatic injector of claim 1, said piston rod having notches, and further comprising a pawl assembly attached to said housing and in communication with said notches to longitudinally move said piston rod into said barrel before activation of said injector.

21. The automatic injector of claim 1, further comprising a safety tab removably engaged with said activator, said tab arranged to prevent activation of said injector when said tab is engaged with said activator.

22. The automatic injector of claim 1, further comprising an adaptor arranged to provide fluid communication between an interior of a vial and said needle.

23. The automatic injector of claim 22, said adaptor including a spike having a conduit therethrough, said spike arranged to pierce into the vial for communication with the interior of the vial.

24. The automatic injector of claim 22, said adaptor including an elastomeric tube adapted to slide about said needle to seal a path between said adaptor and said needle.

25. The automatic injector of claim 1, further comprising a sleeve-like cartridge that receives and holds said syringe, said cartridge arranged to load within said housing.

26. The automatic injector of claim 25, further comprising a static latch that abuts and holds said cartridge in place after said cartridge is loaded.

27. The automatic injector of claim 25, said syringe including a needle hub attached to said needle and said barrel, said needle hub securing said needle to said barrel.

28. The automatic injector of claim 27, further comprising a clip in said cartridge, said clip including:
a hollow sleeve supporting the needle hub, said sleeve having a first rim towards a proximate end of the needle hub and a second rim towards a distal end of the needle hub, said sleeve adapted to slide longitudinally about the needle hub;
a first set of latches extending longitudinally from said first rim, said first set adapted to prevent axial movement of the needle hub after a delivery of a fluid from the needle;
a second set of latches extending longitudinally from said second rim, said second set adapted to prevent axial movement of said needle hub before and after delivery of the fluid.

29. The automatic injector of claim 28, said first set of latches are formed of a flexible elastic material and include proximate ends extending radially inward to abut the needle hub after the delivery.

30. The automatic injector of claim 29, said second set of latches are formed of a flexible elastic material and include distal ends extending radially outward.

31. The automatic injector of claim 30, at least one of said distal ends including a wide section arranged to prevent axial rotation of the clip, and a narrow section extending radially outward of said wide section.

32. The automatic injector of claim 31, said shell having an inner circumferential wall and an outer circumferential wall, said shell including grooves formed in said inner circumferential wall aligned with said second set of latches to accept said narrow section in a sliding relationship.

33. The automatic injector of claim 32, at least one of said grooves including an aperture extending radially outward through said outer circumferential wall, said aperture arranged to hold said narrow section of said distal end and prevent axial movement of said clip and of the needle hub prior to delivery.

34. The automatic injector of claim 33, at least one of said grooves including a locking slit distal of said aperture and extending radially outward toward said outer circumferential wall, said locking slit arranged to hold a respective one of the distal ends and prevent axial movement of said clip after delivery.

35. The automatic injector of claim 34, further comprising an automatic retracting mechanism arranged to bias the needle hub longitudinally from a first position where a proximate section of the needle hub is inside said clip to a second position where the proximate section is outside said clip, so that the proximate section of said needle hub passes through said first set of latches, said first set abutting the proximate section to prevent axial movement of the proximate section back into said clip and prevent axial movement of the needle hub.

36. The automatic injector of claim 1, said barrel comprising a cartridge having a closed distal end and a proximate end, said needle having a distal end for exposure to the injection site and a proximate end arranged to penetrate said closed distal end of the cartridge and provide fluid communication between the distal end of the needle and the interior of the cartridge, said proximate end of said cartridge arranged to accept said piston rod.

37. The automatic injector of claim 36, said cartridge including a front compartment and a rear compartment, each compartment including a substance from a group consisting of at least one of a liquid, a diluent, a lyophilized drug, a reconstituted drug, a powder, a solid element and a fluid element, wherein a combination of the substance from each compartment forms the fluid for delivery.

38. The automatic injector of claim 37, said cartridge further including a plug separating said front and rear compartments, and a bypass allowing the substances to mix prior to delivery.

39. The automatic injector of claim 36, said housing including a door between said distal and proximate ends for loading said cartridge into said housing.

40. The automatic injector of claim 1, said housing including a set of latches extending longitudinally rearward towards said proximate end and extending radially inward, said latches adapted to abut said syringe and prevent axial movement of said syringe toward said distal end of the housing until after activation of said injector.

41. The automatic injector of claim 1, further comprising a pressure sensitive sleeve slidingly holding said syringe and biased to extend beyond said distal end of said housing to a first position, said sleeve adapted to move axially rearward upon the application of rearward pressure applied to said sleeve to a second position.

42. The automatic injector of claim 41, said activation occurring only after application of the axial pressure and then depression of said activator during continued application of the pressure, said sleeve moving to separate said latches from said syringe to allow axial movement of said syringe toward said distal end of said housing.

43. The automatic injector of claim 41, said activator having a projection aligned with a notch in said sleeve when said sleeve is in the first position to prevent axial movement of said sleeve when said activator is depressed, said projection out of alignment with said notch when said sleeve is in the second position to allow axial movement of said sleeve when said activator is depressed.

44. The automatic injector of claim 1, further comprising a holding latch, said holding latch engaging said housing prior to an activation of said injector, and said holding latch disengaging said housing and communicating with said syringe to move said needle to the injection site upon activation of said injector.

45. The automatic injector of claim 1, the control unit including a projection for locking the syringe after delivery.

46. The automatic injector of claim 1, said housing further including a window arranged to allow viewing of the fluid.

47. The automatic injector of claim 1, said syringe further including a flange, said control unit arranged to pivot and engage said control unit with said flange for extending said needle out of said housing, said control unit further arranged to pivot and disengage said control unit from said flange and engage said control unit with said piston rod.

48. An automatic injector for delivering a fluid, comprising:
a housing including means for activating said injector, said housing having a proximate end and a distal end;
a syringe positioned within said housing, said syringe having a needle extending towards said distal end, a barrel, and a piston rod, said barrel arranged to contain fluid therein, the fluid in communication with said needle, said piston rod slidingly located within said barrel for forcing the fluid through said needle upon activation of said injector;
control means for engaging said piston rod and sliding within said housing during delivery and for pivoting out of engagement with said piston rod at the end of delivery; and
driving means for moving said piston rod through said barrel, via said control means, for moving the fluid through said needle for delivery into an injection site.

49. The automatic injector of claim 48, said control means pivoting into engagement with said piston rod before delivery, and sliding towards said distal end during delivery.

50. An automatic injector for titration and delivery of a fluid, comprising:
a housing including an activator arranged to activate said injector, said housing having a proximate end and a distal end;
a syringe positioned within said housing, said syringe having a needle extending towards said distal end, a barrel, and a piston rod, said barrel arranged to contain the fluid therein, the fluid in communication with said needle, said piston rod slidingly located within said barrel for forcing the fluid through said needle upon activation of said injector;
a displacer arranged to communicate with said piston rod before the activation, said displacer arranged to move said piston rod for titration before the activation and to automatically separate from said piston rod upon activation; and
a driving unit in communication with said housing and in communication with said piston rod, said driving unit arranged to move said piston rod through said barrel upon the activation to push the fluid through said needle for delivery into an injection site.

51. The automatic injector of claim 50, wherein said displacer is a back rod.

52. The automatic injector of claim 51, said piston rod having an aperture, said back rod arranged to extend into said aperture during communication with said piston rod and rotate said piston rod during titration.

53. The automatic injector of claim 51, said housing further comprising an opening at said proximate end, said back rod extending through said opening.

54. The automatic injector of claim 53, said back rod having a threaded edge for rotational axial movement in relation to said opening.

55. The automatic injector of claim 53, said back rod having a serrated edge for incremental axial movement in relation to said opening.

56. The automatic injector of claim 55, further comprising a pawl for engagement with said serrated edge, said pawl arranged to allow movement of said back rod towards said piston rod and prevent movement of said back rod away from said piston rod before the activation.

57. The automatic injector of claim 53, said back rod arranged to pivot about said opening to attach to said piston rod, said attachment allowing said back rod to push said piston rod into said barrel and to pull said piston rod from said barrel to draw the fluid into said barrel before the activation.

58. The automatic injector of claim 50, said housing further including a window arranged to allow viewing of the fluid.

59. The automatic injector of claim 50, wherein said syringe has a first longitudinal axis, and said driving unit has a second longitudinal axis different than said first longitudinal axis.

60. The automatic injector of claim 50, said syringe including a needle hub attached to said needle and said barrel, said needle hub securing said needle to said barrel.

61. The automatic injector of claim 60, further comprising a clip in said housing, said clip including:
a hollow sleeve supporting the needle hub, said sleeve having a first rim towards a proximate end of the needle hub and a second rim towards a distal end of the needle hub, said sleeve adapted to slide longitudinally about the needle hub;
a first set of latches extending longitudinally from said first rim, said first set adapted to prevent axial movement of the needle hub after a delivery of a fluid from the needle;
a second set of latches extending longitudinally from said second rim, said second set adapted to prevent axial movement of said needle hub before and after delivery of the fluid.

62. The automatic injector of claim 61, further comprising a bumper located within said housing at said distal end, said bumper arranged to absorb impact between said barrel and said housing.

63. An automatic injector for titration and delivery of a fluid, comprising:
a housing including an activator arranged to activate said injector, said housing having a proximate end and a distal end;
a syringe positioned within said housing, said syringe having a needle extending towards said distal end, a barrel, and a piston rod, said barrel arranged to contain the fluid therein, the fluid in communication with said needle, said piston rod slidingly located within said barrel for forcing the fluid through said needle upon activation of said injector;
titration means for moving said piston rod into said barrel before the activation to expel undesired air or fluid from the syringe and for automatically separating from said piston rod upon the activation; and
driving means for moving said piston rod through said barrel upon the activation to push the fluid through said needle for delivery into an injection site.

64. An automatic injector for titration and delivery of a fluid, comprising:
a housing including an activator arranged to activate said injector, said housing having a proximate end and a distal end;
a syringe positioned within said housing, said syringe having a needle extending towards said distal end, a barrel, and a piston rod, said barrel arranged to contain the fluid therein, the fluid in communication with said needle, said piston rod slidingly located within said barrel for forcing the fluid through said needle upon activation of said injector;
a displacer arranged to communicate with said piston rod before the activation, said displacer arranged to rotate to move said piston rod for titration before the activation, said displacer having an axial sliding relationship with said piston rod during titration; and
a driving unit in communication with said housing and in communication with said piston rod, said driving unit arranged to move said piston rod through said barrel upon the activation to push the fluid through said needle for delivery into an injection site.

65. A needle-locking device for preventing exposure of a needle held by a needle hub, comprising:
a hollow sleeve supporting the needle hub, said sleeve having a first rim towards a proximate end of the needle hub and a second rim towards a distal end of the needle hub, said sleeve adapted to slide longitudinally about the needle hub;
a first set of latches extending longitudinally from said first rim, said first set adapted to prevent axial movement of the needle hub after a delivery of a fluid from the needle; and
a second set of latches extending longitudinally from said second rim, said second set adapted to prevent axial movement of said needle hub before and after delivery of the fluid.

66. The needle-locking device of claim 65, said first set of latches are formed of a flexible elastic material and include proximate ends extending radially inward to abut the needle hub after the delivery.

67. The needle-locking device of claim 65, said second set of latches are formed of a flexible elastic material and include distal ends extending radially outward.

68. The needle-locking device of claim 67, at least one of said distal ends including a wide section arranged to prevent axial rotation of the clip, and a narrow section extending radially outward of said wide section.

69. The needle-locking device of claim 68, further comprising a cartridge shell housing said clip, said shell having an inner circumferential wall and an outer circumferential wall, said shell including grooves formed in said inner circumferential wall of said shell and aligned with said second set of latches to accept said narrow section in a sliding relationship.

70. The needle-locking device of claim 69, at least one of said grooves including an aperture extending radially outward through said outer circumferential wall, said aperture arranged to hold said narrow section of said distal end and prevent axial movement of said clip and of the needle hub prior to delivery.

71. The needle-locking device of claim 70, at least one of said grooves including a locking slit distal of said aperture and extending radially outward toward said outer circumferential wall, said locking slit arranged to hold a respective one of the distal ends and prevent axial movement of said clip after delivery.

72. The needle-locking device of claim 71, further comprising a retraction unit arranged to bias the needle hub longitudinally from a first position where a proximate section of the needle hub is inside said clip to a second position where the proximate section is outside said clip, so that the proximate section of said needle hub passes through said first set of latches, said first set abutting the proximate section to prevent axial movement of the proximate section back into said clip and prevent axial movement of the needle hub.

73. The needle-locking device of claim 65, further comprising a cartridge shell housing said clip, said shell having an inner circumferential wall and an outer circumferential wall, said shell including grooves formed in said inner circumferential wall of said shell and aligned with said second set of latches to accept said second set in a sliding relationship.

74. The needle-locking device of claim 73, at least one of said grooves including an aperture extending radially outward through said outer circumferential wall, said aperture arranged to hold a respective one of said second set of latches and prevent axial movement of said clip and of the needle hub prior to delivery.

75. The needle-locking device of claim 73, at least one of said grooves including a locking slit extending radially outward toward said outer circumferential wall, said locking slit arranged to hold a respective one of said second set of latches and prevent axial movement of said clip after delivery.

76. The needle-locking device of claim 75, further comprising a retraction unit arranged to bias the needle hub longitudinally from a first position where a proximate section of the needle hub is inside said clip to a second position where the proximate section is outside said clip, so that the proximate section of said needle hub passes through said first set of latches, said first set abutting the proximate section to prevent axial movement of the proximate section back into said clip and prevent axial movement of the needle hub.

77. The needle-locking device of claim 65, further comprising an adaptor arranged to provide fluid communication between an interior of a vial and said needle.

78. The needle-locking device of claim 77, said adaptor including a spike having a conduit therethrough, said spike arranged to pierce into the vial for communication with the interior of the vial.

79. The needle-locking device of claim 77, said adaptor including an elastomeric tube adapted to slide about said needle to seal a path between said adaptor and said needle.
